(19) United States
(12) Patent Application Publication
(10) Pub. No.: US 2026/0078180 A1
(43) Pub. Date: Mar. 19, 2026

(54) ANTI-PAR2 ANTIBODIES AND USES THEREOF

(71) Applicant: MEDIMMUNE LIMITED, Cambridge (GB)

(72) Inventors: Claire Dobson, Cambridge (GB); Richard Williams, Cambridge (GB); Ian Gurrell, Cambridge (GB); Sadhana Podichetty, Cambridge (GB); David Fairman, Cambridge (GB); Peter Thornton, Cambridge (GB); Philip Newton, Cambridge (GB)

(21) Appl. No.: 19/334,502

(22) Filed: Sep. 19, 2025

Related U.S. Application Data
(63) Continuation of application No. 18/165,614, filed on Feb. 7, 2023, now abandoned.
Application No. 18/165,614 is a continuation of application No. 17/035,956, filed on Sep. 29, 2020, now Pat. No. 11,591,389.
Application No. 17/035,956 is a division of application No. 15/923,374, filed on Mar. 16, 2018, now Pat. No. 10,836,822.
(60) Provisional application No. 62/472,462, filed on Mar. 16, 2017.
(60) Provisional application No. 62/637,766, filed on Mar. 2, 2018.

(51) Int. Cl. C07K 16/28; A61K 39/00; A61P 29/00

(52) U.S. Cl. CPC C07K 16/28; A61P 29/00; A61K 2039/505; C07K 2317/33; C07K 2317/565; C07K 2317/622; C07K 2317/76; C07K 2317/92; C07K 2317/94

(57) ABSTRACT

The present disclosure provides antibodies and antigen-binding fragments capable of binding PAR2. In some embodiments, the anti-PAR2 antibodies or antigen-binding fragments thereof bind PAR2 in a pH-dependent manner. The disclosure further provides methods for making and using the antibodies and antigen-binding fragments.

### CROSS-REFERENCE TO RELATED APPLICATION

This application is a continuation application of U.S. patent application Ser. No. 18/165,614 filed Feb. 7, 2023, which is a continuation of U.S. patent application Ser. No. 17/035,956 filed Sep. 29, 2020, now U.S. Pat. No. 11,591,389, which is a divisional application of U.S. patent application Ser. No. 15/923,374, filed Mar. 16, 2018, now U.S. Pat. No. 10,836,822, which claims the benefit of priority from U.S. Provisional Application No. 62/637,766, filed on Mar. 2, 2018, and from U.S. Provisional Application No. 62/472,462, filed on Mar. 16, 2017. The foregoing applications are incorporated herein by reference in their entirety.

### REFERENCE TO SEQUENCE LISTING

This application incorporates by reference a Sequence Listing submitted electronically with the application as an XML file entitled “70001C_SeqListing.xml” created on Sep. 19, 2025, and having a size of 801,867 bytes.

### BACKGROUND OF THE DISCLOSURE

Chronic pain is a condition that can affect anyone and imposes a burden on patients, health care systems, and economies. Approximately 100 million people in the United States suffer from chronic pain and the total annual incremental cost of health care due to pain, including medical costs and the economic costs of lost time and wages, is estimated to be between $560 and $635 billion dollars (Institute of Medicine of The National Academies, 2011). Yet, in a survey of chronic pain sufferers, more than half felt they had little to no control over their pain (2006 Voices of Chronic Pain Survey, American Pain Foundation). Pain can be caused by a variety of conditions and diseases, from cancer, to diabetes, to arthritis, and can be classified into categories: nociceptive, neuropathic, and mixed type pain. Nociceptive pain is defined by stimulation of nerve fibers (e.g. by thermal, mechanical, or chemical stimuli) while neuropathic pain is pain caused by diverse causes such as nerve damage, diseases, and, importantly, inflammation. Inflammation, the process by which organisms recruit immune cells and release immune factors to the site of an injury or infection, can thus be both a helpful process of damage repair and a cause of pain.

Many treatments for pain inhibit inflammation. Two common classes of anti-inflammatory pain therapeutics are steroidal anti-inflammatory drugs and non-steroidal anti-inflammatory drugs (NSAIDs). Steroidal anti-inflammatory drugs typically suppress prostaglandins and leukotrienes, the products of inflammation. Such drugs are reliable and potent, but carry the risk of severe side effects, including, for example, reduced bone density, weight fluctuations, immune system suppression, and growth/puberty irregularities (Irving, P. M. et al. (2007) Aliment Pharmacol Ther. 26 (3): 313-329; Goodman et al. J Am Acad Orthop Surg. 2007 August; 15 (8): 450-60). NSAIDs inhibit cyclooxygenase-1 and/or 2 (COX-1 and/or COX-2), which themselves catalyze the reaction of arachidonic acid into prostaglandins. Chronic pain and inflammation can require prolonged treatment, and prolonged inhibition of COX enzymes can lead to gastrointestinal tract problems, such as gastric bleeding and ulcers. Given the risks associated with such anti-inflammation pain treatments, there is a need for alternative approaches for treating pain.

G-Protein Coupled Receptors (GPCRs) are a family of membrane proteins that share a common structural motif of seven transmembrane domains connecting an N-terminal extracellular domain and a C-terminal intracellular domain (Granier et al, Nat Chem Biol. 2012 August; 8 (8): 670-673). GPCRs sense extracellular signals such as photons, hormones, chemokines, etc. and activate intracellular G proteins. Many families of GPCRs exist, such as the Frizzled, Rhodopsin, Secretin, Adhesion, and protease activated receptor (PAR) families (Zhang et al. Nature. 2012 Dec. 20; 492 (7429): 387-392; Zhang et al. Mol Cells. 2015 October; 38 (10): 836-42). While the various GPCR families share overall structural features, they exhibit different functions, bind to different ligands, and are activated by different mechanisms. Activation of the PAR family of GPCRs has been associated with inflammation and nociception (Gieseler et al Cell Commun Signal. 2013; 11:86).

Four PAR receptors have been identified: PAR1, PAR2, PAR3, and PAR4 (Macfarlane et al. Pharmacol Rev. 2001 June; 53 (2): 245-82; Gieseler et al Cell Commun Signal. 2013; 11:86). PAR2 activation has been shown to amplify inflammation and nociception, making its inhibition an attractive target for anti-inflammatory pain therapies. PARs, unlike other GPCRs, are activated by proteolytic cleavage of their extracellular domains, which reveals an N-terminal sequence that acts as a tethered-activating ligand. PAR2, in particular, is cleaved and activated by trypsin and tryptase.

PAR2 expression has been detected in vascularized tissues, airways, osteoblasts, cardiovascular tissue, keratinocytes, exocrine glands, leukocytes, mast cells, intestinal epithelium, kidney, neurons, pancreas, and a variety of smooth muscle types (Macfarlane supra). PAR2 has also been implicated in a variety of diseases or conditions associated with neurogenic inflammation, nociception and transmission of pain. PAR2 may be activated by several host and pathogen-derived serine proteases (e.g., trypsin, mast cell tryptase, tissue kallikreins, or members of the coagulation cascade TF-FVIIa and FVa-FXa).

Monoclonal antibodies have been shown to be useful in a variety of therapeutic applications and many antibody therapeutics are currently on the market (Maggon, Curr Med Chem. 2007; 14 (18): 1978-87; Brekke and Sandlie, Nat Rev Drug Discov 2:52-62, 2003). The most common type of antibody in circulation in the blood stream is immunoglobulin G (IgG). The usefulness of an IgG antibody for therapeutic purposes depends on several factors, including the specificity of the antibody for its target, the strength of its binding to the target, as well as how efficiently the antibody can be produced and how quickly the antibody is cleared from the serum (the serum half-life of the antibody). The serum half-lives of antibodies are frequently regulated by FcRn (neonatal Fc receptor), which binds to the Fc domain of immunoglobulin G (IgG). In vivo, IgGs are thought to be taken up non-specifically by fluid-phase pinocytosis (Pyzik et al, J Immunol. 2015 May 15; 194 (10): 4595-603). Once in the endosome, IgG binds to FcRn, which sorts the IgG into recycling endosomes and back to the cell surface, away from lysosomes and away from degradation. While the recycling rate of IgG has been estimated to be 44% of the fractional catabolic rate, antibody therapeutics can still be depleted in a matter of days post-administration (Kim, Jonghan, et al. Clin. Immunol. 122.2 (2007): 146-155).

Pain associated with inflammation is often a chronic condition. Minimizing the dosage and the frequency of administration of a therapeutic molecule is desirable. Thus there is a need for new anti-inflammation pain therapeutics. Standard monoclonal antibodies are attractive candidates, but in some cases can be limited by their serum half-lives. As such, alternative treatments may be desired.

### BRIEF SUMMARY OF THE DISCLOSURE

The present disclosure provides antibodies that bind PAR2. The antibodies of the disclosure are useful, inter alia, for inhibiting PAR2-mediated signaling and for treating diseases and disorders caused by or related to PAR2 activity and/or signaling.

In some embodiments, the disclosure provides for antibodies or antigen-binding fragments thereof that bind to PAR2 with a greater affinity at pH 7.4 than at pH 6.0. In some embodiments, the antibody or antigen-binding fragment binds to PAR2 with at least 5, 10, 20, 30, 40, 50, 60, 70, 80, 90 or 100 times greater affinity at pH 7.4 than at pH 6.0. In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 3, but wherein 1, 2, 3, 4, or 5 amino acid substitutions, deletions or insertions are optionally present in the sequence of SEQ ID NO: 3; ii) a VH-CDR2 having the amino acid sequence of SEQ ID NO: 4, but wherein 1, 2, 3, 4, or 5 amino acid substitutions, deletions or insertions are optionally present in the sequence of SEQ ID NO: 4; and iii) a VH-CDR3 having the amino acid sequence of SEQ ID NO: 5, but wherein 1, 2, 3, 4, or 5 amino acid substitutions, deletions or insertions are optionally present in the sequence of SEQ ID NO: 5; and wherein the VL comprises: i) a VL-CDR 1 having the amino acid sequence of SEQ ID NO: 8; but wherein 1, 2, 3, 4, or 5 amino acid substitutions, deletions or insertions are optionally present in the sequence of SEQ ID NO: 8; ii) a VL-CDR2 having the amino acid sequence of SEQ ID NO: 9, but wherein 1, 2, 3, 4, or 5 amino acid substitutions, deletions or insertions are optionally present in the sequence of SEQ ID NO: 9; and iii) a VL-CDR3 having the amino acid sequence of SEQ ID NO: 10; but wherein 1, 2, 3, 4, or 5 amino acid substitutions, deletions or insertions are optionally present in the sequence of SEQ ID NO: 10; wherein the amino acid substitutions, deletions or insertions reduce the binding affinity of the antibody or antigen-binding fragment thereof for human PAR2 by no more than 1000, 800, 700, 500, 400, 300, 200, 100, 50 or 10-fold as compared to an antibody or antigen-binding fragment having a VH with an amino acid sequence of SEQ ID NO: 2 and VL with an amino acid sequence of SEQ ID NO: 7 when tested at a pH of 7.4 in a PAR2 binding assay. In some embodiments, the amino acid substitutions, deletions or insertions comprise a homologous substitution. In some embodiments, the antibody or antigen-binding fragment has no more than 1, 2, 3, 4, 5, 6, 7, 8, 9, or 10 amino acid substitutions, insertions or deletions in the VH CDRs as compared to the CDR amino acid sequences present in the sequence of SEQ ID NO: 2. In some embodiments, the antibody or antigen-binding fragment has no more than 1, 2, 3, 4, 5, 6, 7, 8, 9, or 10 amino acid substitutions, insertions or deletions in the VL CDRs as compared to the CDR amino acid sequences present in the sequence of SEQ ID NO: 7. In some embodiments, the 1, 2, 3, 4, 5, 6, 7, 8, 9, or 10 amino acid substitutions, insertions or deletions is 1, 2, 3, 4, 5, 6, 7, 8, 9, or 10 substitutions with a histidine.

In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 3; ii) a VH-CDR2 having the amino acid sequence of SEQ ID NO: 4, but wherein a histidine is optionally present at any one or more of the amino acid positions corresponding to positions 1-17 (e.g., positions 4, 5, and 7-17) of SEQ ID NO: 4; and iii) a VH-CDR3 having the amino acid sequence of SEQ ID NO: 5, but wherein a histidine is optionally present at any one or more of the amino acid positions corresponding to positions 1-8 of SEQ ID NO: 5; and wherein the VL comprises: i) a VL-CDR1 having the amino acid sequence of SEQ ID NO: 8; ii) a VL-CDR2 having the amino acid sequence of SEQ ID NO: 9; and iii) a VL-CDR3 having the amino acid sequence of SEQ ID NO: 10; but wherein a histidine is optionally present at any one or more of the amino acid positions corresponding to positions 1-14 of SEQ ID NO: 10. In some embodiments, the VH comprises: i) a VH-CDR 1 having the amino acid sequence of SEQ ID NO: 3, ii) a VH-CDR2 having the amino acid sequence of SEQ ID NO: 4, iii) a VH-CDR3 having the amino acid sequence of SEQ ID NO: 5, and the VL comprises: i) a VL-CDR1 having the amino acid sequence of SEQ ID NO: 8, ii) a VL-CDR2 having the amino acid sequence of SEQ ID NO: 9, iii) a VL-CDR3 having the amino acid sequence of SEQ ID NO: 10. In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 13, ii) a VH-CDR2 having the amino acid sequence of SEQ ID NO: 14, iii) a VH-CDR3 having the amino acid sequence of SEQ ID NO: 15, and wherein the VL comprises: i) a VL-CDR1 having the amino acid sequence of SEQ ID NO: 18, ii) a VL-CDR2 having the amino acid sequence of SEQ ID NO: 19, iii) a VL-CDR3 having the amino acid sequence of SEQ ID NO: 20. In some embodiments, a histidine is present at the amino acid positions corresponding to positions 5, 8, 12, 16, and 17 of SEQ ID NO: 4; and wherein a histidine is present at the amino acid positions corresponding to positions 2 and 3 of SEQ ID NO: 5. In some embodiments, a histidine is present at the amino acid position corresponding to position 5 of SEQ ID NO: 4. In some embodiments, a histidine is present at the amino acid position corresponding to position 7 of SEQ ID NO: 4. In some embodiments, a histidine is present at the amino acid position corresponding to position 8 of SEQ ID NO: 4. In some embodiments, a histidine is present at the amino acid position corresponding to position 12 of SEQ ID NO: 4. In some embodiments, a histidine is present at the amino acid position corresponding to position 15 of SEQ ID NO: 4. In some embodiments, a histidine is present at the amino acid position corresponding to position 16 of SEQ ID NO: 4. In some embodiments, a histidine is present at the amino acid position corresponding to position 17 of SEQ ID NO: 4. In some embodiments, a histidine is present at the amino acid positions corresponding to positions 5 and 8 of SEQ ID NO: 4. In some embodiments, a histidine is present at the amino acid positions corresponding to positions 5, 8, 12, and 16 of SEQ ID NO: 4. In some embodiments, a histidine is present at the amino acid positions corresponding to positions 5, 8, 12, 16 and 17 of SEQ ID NO: 4. In some embodiments, a histidine is present at the amino acid position corresponding to position 2 of SEQ ID NO: 5. In some embodiments, a histidine is present at the amino acid position corresponding to position 3 of SEQ ID NO: 5. In some embodiments, a histidine is present at the amino acid positions corresponding to positions 2 and 3 of SEQ ID NO: 5. In some embodiments, a histidine is present at the amino acid position corresponding to position 1 of SEQ ID NO: 10. In some embodiments, a histidine is present at the amino acid position corresponding to position 5 of SEQ ID NO: 10. In some embodiments, a histidine is present at the amino acid position corresponding to position 6 of SEQ ID NO: 10. In some embodiments, a histidine is present at the amino acid position corresponding to position 14 of SEQ ID NO: 10. In some embodiments, the VH-CDR2 comprises an amino acid sequence selected from the group consisting of: SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714, 724, 734, 744, 754, 764, 774, 784, 794, and 811-818. In some embodiments, the VH-CDR3 comprises an amino acid sequence selected from the group consisting of: SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, and 819-820. In some embodiments, the VL-CDR3 comprises an amino acid sequence selected from the group consisting of: SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790 and 800. In some embodiments, the VH-CDR2 comprises an amino acid sequence corresponding to SEQ ID NO: 14; wherein the VH-CDR3 comprises an amino acid sequence corresponding to SEQ ID NO: 15, and wherein the VL-CDR3 comprises an amino acid sequence corresponding to SEQ ID NO: 20. In some embodiments, the VH-CDR2 comprises an amino acid sequence corresponding to SEQ ID NO: 811; the VH-CDR3 comprises an amino acid sequence corresponding to SEQ ID NO: 819, and the VL-CDR3 comprises an amino acid sequence corresponding to SEQ ID NO: 10 or 20. In some embodiments, the VH-CDR2 comprises an amino acid sequence corresponding to SEQ ID NO: 814; the VH-CDR3 comprises an amino acid sequence corresponding to SEQ ID NO: 820, and the VL-CDR3 comprises an amino acid sequence corresponding to SEQ ID NO: 10 or 20. In some embodiments, the VH-CDR2 comprises an amino acid sequence corresponding to SEQ ID NO: 816; the VH-CDR3 comprises an amino acid sequence corresponding to SEQ ID NO: 15, and the VL-CDR3 comprises an amino acid sequence corresponding to SEQ ID NO: 10 or 20. In some embodiments, the VH-CDR2 comprises an amino acid sequence corresponding to SEQ ID NO: 818; the VH-CDR3 comprises an amino acid sequence corresponding to SEQ ID NO: 15, and the VL-CDR3 comprises an amino acid sequence corresponding to SEQ ID NO: 10 or 20. In some embodiments, the VH comprises framework regions that are at least 90% identical to each of SEQ ID NOs: 803-806. In some embodiments, the VH comprises framework regions that are at least 95% identical to each of SEQ ID NOs: 803-806. In some embodiments, the VH comprises framework regions corresponding to SEQ ID NOs: 803-806. In some embodiments, the VL comprises framework regions that are at least 90% identical to each of SEQ ID NOs: 807-810. In some embodiments, the VL comprises framework regions that are at least 95% identical to each of SEQ ID NOs: 807-810. In some embodiments, the VL comprises framework regions corresponding to SEQ ID NOs: 807-810. In some embodiments, the VH comprises an amino acid sequence that is at least 80%, 85%, 90%, 92%, 93%, 95%, 97%, 98%, 99% or 100% identical to a sequence selected from the group consisting of: SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, and 792. In some embodiments, the VL comprises an amino acid sequence that is at least 80%, 85%, 90%, 92%, 93%, 95%, 97%, 98%, 99% or 100% identical to a sequence selected from the group consisting of: SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797. In some embodiments, the VH comprises an amino acid sequence that is at least 80%, 85%, 90%, 92%, 93%, 95%, 97%, 99% or 100% identical to SEQ ID NO: 12 and wherein the VL comprises an amino acid sequence that is at least 80%, 85%, 90%, 92%, 93%, 95%, 97%, 99% or 100% identical to SEQ ID NO: 17. In some embodiments, the VH comprises the amino acid sequence of SEQ ID NO: 12, and wherein the VL comprises the amino acid sequence of SEQ ID NO: 17. In some embodiments, the VH comprises an amino acid sequence corresponding to SEQ ID NO: 821 and the VL comprises an amino acid sequence corresponding to SEQ ID NO: 7 or 17. In some embodiments, the VH comprises an amino acid sequence corresponding to SEQ ID NO: 824 and the VL comprises an amino acid sequence corresponding to SEQ ID NO: 7 or 17. In some embodiments, the VH comprises an amino acid sequence corresponding to SEQ ID NO: 827 and the VL comprises an amino acid sequence corresponding to SEQ ID NO: 7 or 17. In some embodiments, the VH comprises an amino acid sequence corresponding to SEQ ID NO: 831 and the VL comprises an amino acid sequence corresponding to SEQ ID NO: 7 or 17. In some embodiments, the antibody or antigen-binding fragment is an antigen-binding fragment. In some embodiments, the antigen-binding fragment is an scFv. In some embodiments, the antigen-binding fragment is a Fab′. In some embodiments, the antibody or antigen-binding fragment is an antibody. In some embodiments, the antibody is a monoclonal antibody. In some embodiments, the antibody is an IgG antibody. In some embodiments, the antibody or antigen-binding fragment is humanized. In some embodiments, the antibody or antigen-binding fragment is human. In some embodiments, the VH is encoded by a nucleic acid comprising a nucleotide sequence that is at least 90% identity to any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the VH is encoded by a nucleic acid comprising a nucleotide sequence that is at least 95% identity to any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the VH is encoded by a nucleic acid comprising the nucleotide sequence of any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the VL is encoded by a nucleic acid comprising a nucleotide sequence that is at least 90% identity to any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the VL is encoded by a nucleic acid comprising a nucleotide sequence that is at least 95% identity to any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the VL is encoded by a nucleic acid comprising the nucleotide sequence of any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the VH is encoded by a nucleic acid comprising a nucleotide sequence that is at least 90% identity to SEQ ID NO: 11. In some embodiments, the VH is encoded by a nucleic acid comprising a nucleotide sequence that is at least 95% identity to SEQ ID NO: 11. In some embodiments, the VH is encoded by a nucleic acid comprising the nucleotide sequence of SEQ ID NO: 11. In some embodiments, the VL is encoded by a nucleic acid comprising a nucleotide sequence that is at least 90% identity to SEQ ID NO: 16. In some embodiments, the VL is encoded by a nucleic acid comprising a nucleotide sequence that is at least 95% identity to SEQ ID NO: 16. In some embodiments, the VL is encoded by a nucleic acid comprising the nucleotide sequence of SEQ ID NO: 16. In some embodiments, the antibody or antigen-binding fragment binds to PAR2. In some embodiments, the antibody or antigen-binding fragment prevents trypsin, tryptase and/or matriptase from interacting with PAR2. In some embodiments, the antibody or antigen-binding fragment prevents trypsin, tryptase and/or matriptase from cleaving PAR2. In some embodiments, the antibody or antigen-binding fragment prevents cleavage of the PAR2 extracellular domain. In some embodiments, the antibody or antigen-binding fragment inhibits exposure of the tethered ligand. In some embodiments, the antibody or antigen-binding fragment prevents the tethered ligand from interacting with the second transmembrane loop of PAR2. In some embodiments, the antibody or antigen-binding fragment binds to PAR2 with greater affinity at a pH of 7.4 than at a pH of 6.0. In some embodiments, the antibody or antigen-binding fragment has an IC50 of less than 100 nM when competing with an antibody or antigen-binding fragment having the CDRs of SEQ ID NOs: 3-5 and 8-10 at a pH of 7.4 in a PAR2 binding assay. In some embodiments, the antibody or antigen-binding fragment has an IC50 of less than 50 nM when competing with an antibody or antigen-binding fragment having the CDRs of SEQ ID NOs: 3-5 and 8-10 at a pH of 7.4 in a PAR2 binding assay. In some embodiments, the antibody or antigen-binding fragment has an IC50 of less than 40 nM when competing with an antibody or antigen-binding fragment having the CDRs of SEQ ID NOs: 3-5 and 8-10 at a pH of 7.4 in a PAR2 binding assay. In some embodiments, the antibody or antigen-binding fragment has an IC50 of greater than 500 nM when competing with an antibody or antigen-binding fragment having the CDRs of SEQ ID NOs: 3-5 and 8-10 at a pH of 6.0 in a PAR2 binding assay. In some embodiments, the antibody or antigen-binding fragment has an IC50 of greater than 1000 nM when competing with an antibody or antigen-binding fragment having the CDRs of SEQ ID NOs: 3-5 and 8-10 at a pH of 6.0 in a PAR2 binding assay. In some embodiments, the antibody or antigen-binding fragment has an IC50 of greater than 1100 nM when competing with an antibody or antigen-binding fragment having the CDRs of SEQ ID NOs: 3-5 and 8-10 at a pH of 6.0 in a PAR2 binding assay. In some embodiments, the antibody or antigen-binding fragment has an IC50 more than 20 times lower at a pH of 7.4 than at a pH of 6.0 when competing with an antibody or antigen-binding fragment having the CDRs of SEQ ID NOs: 3-5 and 8-10 in a PAR2 binding assay. In some embodiments, the antibody or antigen-binding fragment has an IC50 more than 25 times lower at a pH of 7.4 than at a pH of 6.0 when competing with an antibody or antigen-binding fragment having the CDRs of SEQ ID NOs: 3-5 and 8-10 in a PAR2 binding assay. In some embodiments, the antibody or antigen-binding fragment has an IC50 more than 30 times lower at a pH of 7.4 than at a pH of 6.0 when competing with an antibody or antigen-binding fragment having the CDRs of SEQ ID NOs: 3-5 and 8-10 in a PAR2 binding assay. In some embodiments, the antibody or antigen-binding fragment has an IC50 of less than 3.0×10⁻¹⁰ M in a calcium influx assay in human A549 cells. In some embodiments, the antibody or antigen-binding fragment has an IC50 of less than 1.5×10⁻¹⁰ M in a calcium influx assay in human A549 cells. In some embodiments, the antibody or antigen-binding fragment has an IC50 of less than 7.0×10⁻¹⁰ M in a calcium influx assay in rat KNRK cells. In some embodiments, the antibody or antigen-binding fragment has an IC50 of less than 5.5×10⁻¹⁰ M in a calcium influx assay in rat KNRK cells. In some embodiments, the antibody or antigen-binding fragment has an IC50 of less than 7.0×10⁻¹¹ M in a calcium influx assay in cynomolgus monkey CYNOM-K1 cells. In some embodiments, the antibody or antigen-binding fragment has an IC50 of less than 5.0×10⁻¹¹ M in a calcium influx assay in cynomolgus monkey CYNOM-K1 cells. In some embodiments, the antibody or antigen-binding fragment has an IC50 of less than 6.0×10⁻¹¹ M in a calcium influx assay in murine LL/2 cells. In some embodiments, the antibody or antigen-binding fragment has an IC50 of less than 4.0×10⁻¹¹ M in a calcium influx assay in murine LL/2 cells. In some embodiments, the antibody or antigen-binding fragment is cleared more slowly from serum of a treated patient than an antibody or antigen-binding fragment lacking histidine modifications. In some embodiments, the optionally present histidine or histidines reduce the binding affinity of the antibody or antigen-binding fragment thereof for human PAR2 by no more than 1000, 800, 700, 500, 400, 300, 200, 100, 50 or 10-fold as compared to an antibody or antigen-binding fragment having a VH with an amino acid sequence of SEQ ID NO: 2 and VL with an amino acid sequence of SEQ ID NO: 7 when tested at a pH of 7.4 in a PAR2 binding assay.

In some embodiments, the disclosure provides for a nucleic acid capable of expressing any of the antibodies or antigen-binding fragments disclosed herein. In some embodiments, the nucleic acid comprises a nucleotide sequence that is at least 90% identical to any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the nucleic acid comprises a nucleotide sequence that is at least 95% identical to any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the nucleic acid comprises the nucleotide sequence of any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the disclosure provides for a nucleic acid comprising a nucleotide sequence that is at least 90% identical to any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the nucleic acid comprises a nucleotide sequence that is at least 95% identical to any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the nucleic acid comprises the nucleotide sequence of any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the disclosure provides for a nucleic acid comprising a nucleotide sequence that is at least 90% identical to SEQ ID NO: 11. In some embodiments, the nucleic acid comprises a nucleotide sequence that is at least 95% identical to SEQ ID NO: 11. In some embodiments, the nucleic acid comprises the nucleotide sequence of SEQ ID NO: 11. In some embodiments, the disclosure provides for a nucleic acid comprising a nucleotide sequence that is at least 90% identical to SEQ ID NO: 16. In some embodiments, the nucleic acid comprises a nucleotide sequence that is at least 95% identical to SEQ ID NO: 16. In some embodiments, the nucleic acid comprises the nucleotide sequence of SEQ ID NO: 16.

In some embodiments, the disclosure provides for a vector comprising any of the the nucleic acids disclosed herein. In some embodiments, the disclosure provides for a set of vectors comprising any one or more of the nucleic acids disclosed herein.

In some embodiments, the disclosure provides for a host cell comprising any one or more of the vectors disclosed herein.

In some embodiments, the disclosure provides for a composition comprising a pharmaceutically acceptable carrier and any of the antibodies or antigen-binding fragments disclosed herein.

In some embodiments, the disclosure provides for a lyophilized composition comprising any of the antibody or antigen-binding fragment thereof disclosed herein.

In some embodiments, the disclosure provides for a reconstituted lyophilized composition comprising any of the antibodies or antigen-binding fragments thereof disclosed herein. In some embodiments, the composition is formulated for administration by lozenge, spray, oral administration, delayed release or sustained release, transmucosal administration, syrup, mucoadhesive, buccal formulation, mucoadhesive tablet, topical administration, parenteral administration, injection, subdermal administration, oral solution, rectal administration, buccal administration or transdermal administration.

In some embodiments, the disclosure provides for a kit comprising any of the antibodies or antigen-binding fragments disclosed herein or any of the compositions disclosed herein.

In some embodiments, the disclosure provides for a method for treating pain in a subject in need thereof, comprising administering to the subject a pharmaceutically effective amount of any of the antibodies or antigen-binding fragments disclosed herein. In some embodiments, the disclosure provides for a method for treating pain in a subject in need thereof, comprising administering to the subject a pharmaceutically effective amount of any of the compositions disclosed herein. In some embodiments, the pain is selected from the group consisting of: nociceptive, neuropathic, and mix-type pain. In some embodiments, the pain is associated with a headache, chronic headache, a migraine headache, a cancer, a viral infection, rheumatoid arthritis, osteoarthritis, Crohn's disease, liver disease, multiple sclerosis, spinal cord injury, post herpetic neuralgia, diabetic neuropathy, lower back pain, inflammatory heart disease, kidney disease, gastritis, gingivitis, periodontal disease, asthma, chronic obstructive pulmonary disease, autoimmune disease, irritable bowel syndrome, fibromyalgia, leg pains, restless leg syndrome, diabetic neuropathy, an allergic condition, a surgical procedure, acute or chronic physical injury, bone fracture or a crush injury, spinal cord injury, an inflammatory disease, a non-inflammatory neuropathic or dysfunctional pain condition, or a combination thereof. In some embodiments, the pain is osteoarthritis pain. In some embodiments, the subject is a human.

In some embodiments, the disclosure provides for a method of producing any of the antibodies or antigen-binding fragments disclosed herein, comprising the steps of: expressing any of the the nucleic acids disclosed herein in a cultured cell, purifying the antibody or antigen-binding fragment.

### BRIEF DESCRIPTION OF THE DRAWINGS

FIGS. 1A and 1B are tables illustrating the sequence differences in VH VH CDR2 (SEQ ID NOS 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714, 724, 734, 744, 754, 764, 774, 784, and 794, respectively, in order of appearance) and CDR3 (SEQ ID NOS 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, and 795, respectively, in order of appearance) of various clones as compared to the same CDRs of Par0067. CDR 1 and the framework regions are the same for Par0067 and for all of the clones indicated (i.e., Par0067 and each of the clones comprised the sequences of SEQ ID NOs: 3 and 803-806).

FIGS. 2A and 2B are tables illustrating the sequence differences in VL CDR3 (SEQ ID NOS 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, and 800, respectively, in order of appearance) of various clones as compared to the same CDR of Par0067. CDR1, CDR2 and the framework regions are the same for Par0067 and for all of the clones indicated (i.e., the clones comprised the sequences of SEQ ID NOs: 8, 9, and 807-810).

FIGS. 3A-3E provide IC50 data from a cell potency assay using various IgG-based antibodies. The types of cells used in each of the cell assays are indicated. NI=non-inhibitory.

FIG. 4A provides IC50 curves for PaB670129 against trypsin in A549 cells, relative to agonistic responses to the antibody at the equivalent concentrations. FIG. 4B provides IC50 curves for PaB670129 against different PAR2 protease activators.

FIGS. 5A-5F illustrate the results from experiments in which rat dorsal root ganglia (DRG) sensory neurons or non-neuronal cells were treated with matripase in the presence or absence of PaB670129 (also referred to herein as PaB670129). Rat DRG sensory neurones pre-treated with isotype control (20 nM) display matriptase-induced calcium transients (5A). Sensory neurons pre-treated with PaB670129 IgG1TM (20 nM) do not respond to matriptase (5B); % of neurones responding to matriptase quantified in (5C). Non-neuronal cells of the DRG pre-treated with isotype control (20 nM) also display matriptase-induced calcium transients (8D), but non-neuronal cells pre-treated with PaB670129 IgG1TM (20 nM) do not (5E); % of non-neuronal cell responding to matriptase quantified in (5F). FIGS. 5A-5E disclose “LIGRLO” as SEQ ID NO: 832

FIG. 6 illustrates the effects of PaB670129 (versus anti-PAR1 antibodies or Vorapaxar) on thrombin-induced PAR1 activation in human A549 cells.

FIG. 7A depicts a graph illustrating the effect of different treatments (including a PAR2 antibody, Par0067) on the percent ipsilateral/contralateral hypersensitivity induced by mono-iodoacetate (MIA) in rat. FIG. 7B depicts a graph illustrating the effect of different doses of PaB670129 or an isotype control antibody on the percent ipsilateral/contralateral hypersensitivity induced by MIA in rat. “i.v.” means intravenous, and “p.o.” means per os (oral). Statistical analysis-Repeated measures ANOVA, followed by a planned comparison test, using invivostat. Significant hyperalgesia (*** P<0.001) post injection of MIA with vehicle from day 7 to day when compared to baseline and sham. * P<0.05, ** P<0.01, *** P<0.001 Significant reversal of hyperalgesia when compared to vehicle at each time point.

FIG. 8 depicts a graph illustrating the effect of different doses of PaB670129 or an isotype control antibody on the percent ipsilateral/contralateral hypersensitivity induced by peripheral nerve partial ligation in mouse. “s.c.” means sub-cutaneous. N=9-10 per group. Data were analyzed using 2-way ANOVA with time and treatment as dependent factors. Subsequent statistical significance was obtained using Tukey's Post Hoc test. Individual comparisons shown * P<0.05; ** P<0.01; *** P<0.001 vs. Op+isotype control 10 mg/kg.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs.

Although any methods and materials similar or equivalent to those described herein can be used in the practice or testing of the present disclosure, the preferred methods and materials are now described.

### A. Definitions

As used in this specification and the appended claims, the singular form “a”, “an” and “the” include plural referents unless the context clearly dictates otherwise.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The expressions “protease activated receptor 2,” “PAR2,” and the like, as used herein, refer to a human PAR2 protein having an amino acid sequence that is at least 90%, 91%, 92%, 93%, 94%, 95%, 96%, 97%, 98%, 99%, or 100% identical to the amino acid sequence of any of SEQ ID NO: 801, or biologically active fragments thereof.

The term “tethered ligand” refers to a region of the N-terminal portion of PAR2 that binds to and activates the PAR2 receptor itself. In some embodiments, the tethered ligand portion of PAR2 is not exposed until a protease (e.g., thrombin or trypsin) proteolytically cleaves a portion of the PAR2 enzyme. In some embodiments, the tethered ligand corresponds to a polypeptide that is at least 90%, 91%, 92%, 93%, 94%, 95%, 96%, 97%, 98%, 99%, or 100% identical to the amino acid sequence of any of SEQ ID NO: 802

As used herein, “an antibody that binds PAR2”, “anti-PAR2 antibody,” and the like includes antibodies, and antigen-binding fragments thereof, that bind membrane-bound PAR2 or fragments thereof. In some embodiments, an anti-PAR2 antibody or antigen binding fragment thereof binds to the tethered ligand portion of PAR2.

### B. Antibodies and Antigen-Binding Fragments Thereof

As used herein, “antibodies or antigen binding fragments of the disclosure” refer to any one or more of the antibodies and antigen binding fragments provided herein. Antibodies and antigen binding fragments of the disclosure comprise a heavy chain (VH) comprising a heavy chain variable domain and a light chain (VL) comprising a light chain variable domain. A VH domain comprises three CDRs, such as any of the CDRs provided herein and as defined or identified by the Chothia, Kabat or IMGT systems. These CDRs are typically interspersed with framework regions (FR), and together comprise the VH domain. Similarly, a VL comprises three CDRs, such as any of the CDRs provided herein and as defined by the Chothia, Kabat or IMGT systems. These CDRs are typically interspersed with framework regions (FR), and together comprise the VL domain. The FR regions, such as FR1, FR2, FR3, and/or FR4 can similarly be defined or identified by the Chothia, Kabat or IMGT systems. Throughout the application, when CDRs are indicated as being, as identified or as defined by the Chothia, Kabat or IMGT systems, what is meant is that the CDRs are in accordance with that system (e.g., the Chothia CDRs, Kabat CDRs or the IMGT CDRs). Any of these terms can be used to indicate whether the Chothia, Kabat or IMGT CDRs are being referred to.

The term “antibody”, as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

In some embodiments, the disclosure provides for antibodies or antigen-binding fragments thereof that bind to PAR2 with a greater affinity at pH 7.4 than at pH 6.0. In some embodiments, the antibody or antigen-binding fragment binds to PAR2 with at least 5, 10, 20, 30, 40, 50, 60, 70, 80, 90 or 100 times greater affinity at pH 7.4 than at pH 6.0. In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 3, but wherein 1, 2, 3, 4, or 5 amino acid substitutions, deletions or insertions are optionally present in the sequence of SEQ ID NO: 3; ii) a VH-CDR2 having the amino acid sequence of SEQ ID NO: 4, but wherein 1, 2, 3, 4, or 5 amino acid substitutions, deletions or insertions are optionally present in the sequence of SEQ ID NO: 4; and iii) a VH-CDR3 having the amino acid sequence of SEQ ID NO: 5, but wherein 1, 2, 3, 4, or 5 amino acid substitutions, deletions or insertions are optionally present in the sequence of SEQ ID NO: 5; and wherein the VL comprises: i) a VL-CDR 1 having the amino acid sequence of SEQ ID NO: 8; but wherein 1, 2, 3, 4, or 5 amino acid substitutions, deletions or insertions are optionally present in the sequence of SEQ ID NO: 8; ii) a VL-CDR2 having the amino acid sequence of SEQ ID NO: 9, but wherein 1, 2, 3, 4, or 5 amino acid substitutions, deletions or insertions are optionally present in the sequence of SEQ ID NO: 9; and iii) a VL-CDR3 having the amino acid sequence of SEQ ID NO: 10; but wherein 1, 2, 3, 4, or 5 amino acid substitutions, deletions or insertions are optionally present in the sequence of SEQ ID NO: 10; wherein the amino acid substitutions, deletions or insertions reduce the binding affinity of the antibody or antigen-binding fragment thereof for human PAR2 by no more than 1000, 800, 700, 500, 400, 300, 200, 100, 50, 10 or 5-fold as compared to an antibody or antigen-binding fragment having a VH with an amino acid sequence of SEQ ID NO: 2 and VL with an amino acid sequence of SEQ ID NO: 7 when tested at a pH of 7.4 in a PAR2 binding assay. In some embodiments, the amino acid substitutions, deletions or insertions comprise a homologous substitution. In some embodiments, the antibody or antigen-binding fragment has no more than 1, 2, 3, 4, 5, 6, 7, 8, 9, or 10 amino acid substitutions, insertions or deletions in the VH CDRs as compared to the CDR amino acid sequences present in the sequence of SEQ ID NO: 2. In some embodiments, the antibody or antigen-binding fragment has no more than 1, 2, 3, 4, 5, 6, 7, 8, 9, or 10 amino acid substitutions, insertions or deletions in the VL CDRs as compared to the CDR amino acid sequences present in the sequence of SEQ ID NO: 7. In some embodiments, the 1, 2, 3, 4, 5, 6, 7, 8, 9, or 10 amino acid substitutions, insertions or deletions is 1, 2, 3, 4, 5, 6, 7, 8, 9, or 10 substitutions with a histidine.

In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 13, but wherein 1, 2, 3, 4, or 5 amino acid substitutions, deletions or insertions are optionally present in the sequence of SEQ ID NO: 13; ii) a VH-CDR2 having the amino acid sequence of SEQ ID NO: 14, but wherein 1, 2, 3, 4, or 5 amino acid substitutions, deletions or insertions are optionally present in the sequence of SEQ ID NO: 14; and iii) a VH-CDR3 having the amino acid sequence of SEQ ID NO: 15, but wherein 1, 2, 3, 4, or 15 amino acid substitutions, deletions or insertions are optionally present in the sequence of SEQ ID NO: 15; and wherein the VL comprises: i) a VL-CDR1 having the amino acid sequence of SEQ ID NO: 18; but wherein 1, 2, 3, 4, or 5 amino acid substitutions, deletions or insertions are optionally present in the sequence of SEQ ID NO: 18; ii) a VL-CDR2 having the amino acid sequence of SEQ ID NO: 19, but wherein 1, 2, 3, 4, or 5 amino acid substitutions, deletions or insertions are optionally present in the sequence of SEQ ID NO: 19; and iii) a VL-CDR3 having the amino acid sequence of SEQ ID NO: 20; but wherein 1, 2, 3, 4, or 5 amino acid substitutions, deletions or insertions are optionally present in the sequence of SEQ ID NO: 20; wherein the amino acid substitutions, deletions or insertions reduce the binding affinity of the antibody or antigen-binding fragment thereof for human PAR2 by no more than 1000, 800, 700, 500, 400, 300, 200, 100, 50, 10 or 5-fold as compared to an antibody or antigen-binding fragment having a VH with an amino acid sequence of SEQ ID NO: 12 and VL with an amino acid sequence of SEQ ID NO: 17 when tested at a pH of 7.4 in a PAR2 binding assay. In some embodiments, the amino acid substitutions, deletions or insertions comprise a homologous substitution. In some embodiments, the antibody or antigen-binding fragment has no more than 1, 2, 3, 4, 5, 6, 7, 8, 9, or 10 amino acid substitutions, insertions or deletions in the VH CDRs as compared to the CDR amino acid sequences present in the sequence of SEQ ID NO: 12. In some embodiments, the antibody or antigen-binding fragment has no more than 1, 2, 3, 4, 5, 6, 7, 8, 9, or 10 amino acid substitutions, insertions or deletions in the VL CDRs as compared to the CDR amino acid sequences present in the sequence of SEQ ID NO: 17. In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 13, ii) a VH-CDR2 having the amino acid sequence of SEQ ID NO: 14, iii) a VH-CDR3 having the amino acid sequence of SEQ ID NO: 15, and wherein the VL comprises: i) a VL-CDR1 having the amino acid sequence of SEQ ID NO: 18, ii) a VL-CDR2 having the amino acid sequence of SEQ ID NO: 19, iii) a VL-CDR3 having the amino acid sequence of SEQ ID NO: 20. In some embodiments, the 1, 2, 3, 4, 5, 6, 7, 8, 9, or 10 amino acid substitutions, insertions or deletions is 1, 2, 3, 4, 5, 6, 7, 8, 9, or 10 substitutions with a histidine.

In some embodiments, the disclosure provides for an antibody or antigen-binding fragment, wherein the antibody or antigen-binding fragment comprises a light chain variable (VL) domain and a heavy chain variable (VH) domain; wherein the VH domain comprises at least one, two or all three of the CDRs (e.g., as measured using any of the Chothia, IMGT or Kabat systems) of the amino acid sequence set forth in any one of SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, and 792. In some embodiments, the disclosure provides for an antibody or antigen-binding fragment, wherein the antibody or antigen-binding fragment comprises a light chain variable (VL) domain and a heavy chain variable (VH) domain; wherein the VH domain comprises CDR1, CDR2 and CDR3 (e.g., as measured using any of the Chothia, IMGT or Kabat systems) of the amino acid sequence set forth in any one of SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, and 792. In some embodiments, the disclosure provides for an antibody or antigen-binding fragment, wherein the antibody or antigen-binding fragment comprises a light chain variable (VL) domain and a heavy chain variable (VH) domain; wherein the VH domain comprises at least one, two or all three of the CDRs (e.g., as measured using any of the Chothia, IMGT or Kabat systems) of the amino acid sequence set forth in SEQ ID NO: 2 or 12. In some embodiments, the disclosure provides for an antibody or antigen-binding fragment, wherein the antibody or antigen-binding fragment comprises a light chain variable (VL) domain and a heavy chain variable (VH) domain; wherein the VL domain comprises at least one, two or all three of the CDRs (e.g., as measured using any of the Chothia, IMGT or Kabat systems) of the amino acid sequence set forth in any one of SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797. In some embodiments, the disclosure provides for an antibody or antigen-binding fragment, wherein the antibody or antigen-binding fragment comprises a light chain variable (VL) domain and a heavy chain variable (VH) domain; wherein the VL domain comprises CDR1, CDR2 and CDR3 (e.g., as measured using any of the Chothia, IMGT or Kabat systems) of the amino acid sequence set forth in any one of SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797. In some embodiments, the disclosure provides for an antibody or antigen-binding fragment, wherein the antibody or antigen-binding fragment comprises a light chain variable (VL) domain and a heavy chain variable (VH) domain; wherein the VL domain comprises at least one, two or all three of the CDRs (e.g., as measured using any of the Chothia, IMGT or Kabat systems) of the amino acid sequence set forth in SEQ ID NO: 7 or 17. In some embodiments, the disclosure provides for an antibody or antigen-binding fragment, wherein the antibody or antigen-binding fragment comprises a light chain variable (VL) domain and a heavy chain variable (VH) domain; wherein the VH domain comprises CDR1, CDR2 and CDR3 (e.g., as measured using any of the Chothia, IMGT or Kabat systems) of the amino acid sequence set forth in any one of SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, and 792, and wherein the VL domain comprises CDR1, CDR2 and CDR3 (e.g., as measured using any of the Chothia, IMGT or Kabat systems) of the amino acid sequence set forth in any one of SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797. In some embodiments, the disclosure provides for an antibody or antigen-binding fragment, wherein the antibody or antigen-binding fragment comprises a light chain variable (VL) domain and a heavy chain variable (VH) domain; wherein the VH domain comprises CDR1, CDR2 and CDR3 (e.g., as measured using any of the Chothia, IMGT or Kabat systems) of the amino acid sequence set forth in SEQ ID NO: 2 or 12, and wherein the VL domain comprises CDR1, CDR2 and CDR3 (e.g., as measured using any of the Chothia, IMGT or Kabat systems) of the amino acid sequence set forth in SEQ ID NO: 7 or 17.

Once the nucleotide sequences encoding such antibodies have been determined, chimeric or humanized antibodies may be produced by recombinant methods. Nucleic acids encoding the antibodies are introduced into host cells and expressed using materials and procedures generally known in the art, and as disclosed herein. In some embodiments, the VH is encoded by a nucleic acid comprising a nucleotide sequence that is at least 80%, 85%, 90%, 91%, 92%, 93%, 94%, 95%, 96%, 97%, 98%, 99% or 100% identity to any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, 791, and 833-841. In some embodiments, the VH is encoded by a nucleic acid comprising a nucleotide sequence that is at least 80%, 85%, 90%, 91%, 92%, 93%, 94%, 95%, 96%, 97%, 98%, 99% or 100% identity to SEQ ID NO: 1 or 11. In some embodiments, the VL is encoded by a nucleic acid comprising a nucleotide sequence that is at least 80%, 85%, 90%, 91%, 92%, 93%, 94%, 95%, 96%, 97%, 98%, 99% or 100% identity to any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and 796. In some embodiments, the VL is encoded by a nucleic acid comprising a nucleotide sequence that is at least 80%, 85%, 90%, 91%, 92%, 93%, 94%, 95%, 96%, 97%, 98%, 99% or 100% identity to SEQ ID NO: 6 or 16.

The present disclosure includes anti-PAR2 antibodies and antigen-binding fragments thereof that bind PAR2. In some embodiments, the antibody is a neutralizing and/or blocking anti-PAR2 antibody or antigen-binding fragment. A “neutralizing” or “blocking” antibody or antigen-binding fragment, as used herein, is intended to refer to an antibody or antigen-binding fragment whose binding to PAR2: (i) interferes with the interaction between PAR2 and a protease (e.g., trypsin, tryptase and/or matriptase); (ii) inhibits the cleavage of PAR2 by a protease; (iii) inhibits PAR2 signalling or PAR2 activation; and/or (iv) results in inhibition of at least one biological function of PAR2. In some embodiments, the antibodies or antigen-binding fragments of the disclosure inhibit activation of PAR2. In some embodiments, the antibodies or antigen-binding fragments inhibit conversion of inactive, uncleaved PAR2 into active, cleaved PAR2. In some embodiments, the antibodies or antigen-binding fragments inhibit exposure of the tethered ligand. In some embodiments, the antibodies or antigen-binding fragments inhibit activation of a PAR2 receptor by its tethered ligand. In some embodiments, the antibodies or antigen-binding fragments inhibit binding of the tethered ligand to the second transmembrane domain of PAR2. The inhibition caused by an anti-PAR2 neutralizing or blocking antibody need not be complete so long as it is detectable using an appropriate assay. In some embodiments, the antibody or antigen-binding fragment thereof inhibits PAR2 activity at least 10%, 20%, 30%, 40%, 50%, 60%, 70%, 80%, 90% or 100% as compared to uninhibited active PAR2. Some examples of assays for detecting activity of a representative anti-PAR2 antibody or antigen-binding fragment are described in the Exemplification section. The skilled worker is aware of additional anti-PAR2 antibody activity assays.

In particular embodiments, any of the antibodies or antigen-binding fragments disclosed herein interferes with the interaction between PAR2 and a protease. In some embodiments, the protease is trypsin. In some embodiments, the protease is neutrophil elastase. In some embodiments, the protease is neutrophil proteinase 3. In some embodiments, the protease is mast cell tryptase. In some embodiments, the protease is tissue factor/factor VIIa/factor Xa. In some embodiments, the protease is a kallikrein-related peptidase. In some embodiments, the protease is membrane-tethered serine proteinase-1/matriptase 1. In some embodiments, the protease is parasite cysteine proteinase. In some embodiments, the anti-PAR2 antibodies or antigen-binding fragments block the interaction between PAR2 and a protease (e.g., trypsin) in vitro, with an IC50 value of less than about 15 nM, as measured by a binding assay such as that described in the Exemplification section. In certain embodiments, the antibodies or antigen-binding fragments of the present disclosure block the interaction between PAR2 and a protease (e.g., trypsin) in vitro at a pH of 7.4 with an IC50 value of less than about 200 nM, 150 nM, 100 nM, 50 nM, 40 nM, 30 nM, 20 nM, 10 nM, 1 nM, 500 pM, 400 pM, 200 pM, 100 pM, 50 pM, 5 pM, 1 pM or 0.1 pM. In certain embodiments, the antibodies or antigen-binding fragments of the present disclosure block the interaction between PAR2 and a protease (e.g., trypsin) in vitro at a pH of 6.0 with an IC50 value of greater than about 300 nM, 500 nM, 750 nM, 1000 nM, 1100 nM, or 1200 nM. In certain embodiments, the IC₅₀ of the anti-PAR2 antibody or fragment thereof is measured in an epitope competition assay, such as the epitope competition assay described in the Exemplification section provided herein. In some embodiments, the IC₅₀ of the anti-PAR2 antibody or fragment thereof is measured in a cell potency assay. In some embodiments, the cell potency assay utilizes a human cell (e.g., A549 cell), a rat cell (e.g., KNRK cell), a cynomolgus monkey cell (e.g., CYNOM-K1 cell), or a murine cell (e.g., an LL/2 cell). In some embodiments, the cell potency assay utilizes a calcium influx assay (e.g., the calcium influx assay described in the Exemplification section provided herein). In some embodiments, the antibody or antigen-binding fragment inhibits calcium influx in the calcium influx assay with an IC50 of less than InM, 500 pM, 400 pM, 200 pM, 100 pM, 50 pM, 10 pM, 5 pM, 1 pM or 0.1 pM. In some embodiments, the antibodies or antigen-binding fragments prevent abnormal activation of PAR2 by trypsin. In some embodiments, the antibodies or antigen-binding fragments inhibit/reduce inflammation-induced pain.

In particular embodiments, any of the antibodies or antigen-binding fragments disclosed herein interfere with the interaction between PAR2 and a protease (e.g., trypsin). In some embodiments, the antibodies prevent a protease (e.g., trypsin) from binding, cleaving, and/or activating PAR2. The present disclosure provides for anti-PAR2 antibodies and antigen-binding fragments thereof that bind PAR2 molecules with high affinity at physiological, extracellular pH (i.e. pH 7.4). In some embodiments, antibodies and antigen-binding fragments of antibodies bind PAR2 at pH 7.4 (e.g., at 25° C. or 37° C.) with a K_{D} of less than about 5 nM, 1 nM, 900 PM, 800 pM, 700 pM, 650 pM, 600 pM, 500 pM, 200 pM, 100 pM or 50 pM. In some embodiments, antibodies and antigen-binding fragments of antibodies bind PAR2 at a slightly acidic pH (such as pH 6.0) (e.g., at 25° C. or 37° C.) with a K_{D} of greater than about 1 nM, 5 nM, 10 nM, 15 nM, 20 nM, 25 nM, 30 nM, 40 nM, 50 nM, 60 nM, 80 nM, or 100 nM. In some embodiments, the slightly acidic pH is the pH of an endosomal compartment. In some embodiments, K_{D} can be measured in accordance with currently standard methods, such as using Surface Plasmon Resonance (SPR) or Quartz Crystal Microbalance (QCM).

The present disclosure also includes anti-PAR2 antibodies and antigen-binding fragments thereof that specifically bind to PAR2 with a dissociative half-life (t1/2) of greater than about 1.5 minutes, 1.75 minutes, 2 minutes, 2.5 minutes, 3 minutes, 5 minutes, 10 minutes, 20 minutes, or 30 minutes as measured using an assay such as surface plasmon resonance at 25° C. or 37° C. at pH 7.4. In some embodiments, anti-PAR2 antibodies and antigen-binding fragments thereof bind to PAR2 with a dissociative half-life (t½) of less than about 1 minute, 45 seconds, 30 seconds, 20 seconds, 15 seconds, 13 seconds, 7 seconds, 5 seconds, or 3 seconds as measured using an assay such as surface plasmon resonance at 25° C. or 37° C. at a slightly acidic pH (e.g., pH 6). In some embodiments, the slightly acidic pH is the pH of an endosomal compartment. In some embodiments, K_{D} can be measured in accordance with currently standard methods, such as using Surface Plasmon Resonance (SPR) or Quartz Crystal Microbalance (QCM).

The antibodies or antigen-binding fragments of the present disclosure may possess one or more of the aforementioned biological characteristics, or any combinations thereof. Other biological characteristics of the antibodies of the present disclosure will be evident to a person of ordinary skill in the art from a review of the present disclosure including the Exemplification section provided herein.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. In some embodiments, any of the antibodies or antigen-binding fragments disclosed herein comprise 1, 2, 3, 4, 5, 6, 7, 8, 9, 10, 11, 12, 13, 14, 15, 16, 17, 18, 19, or 20 conservative amino acid substitutions as compared to a reference sequence (e.g., any of the the amino acid sequences of SEQ ID NOs: 2, 7, 12 or 17). A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256:1443-1445. A “moderately conservative” replacement is any change having a nonnegative value in the PAM250 log-likelihood matrix.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called a, 8, ¿, Y, and u, respectively. The subunit structures and three-dimensional configurations of different classes of immunoglobulins are well known and described generally in, for example, Abbas et al. Cellular and Mol. Immunology, 4th ed. (W.B. Saunders, Co., 2000). An antibody may be part of a larger fusion molecule, formed by covalent or non-covalent association of the antibody with one or more other proteins or peptides.

Non-limiting examples of antigen-binding fragments include: (i) Fab fragments; (ii) Fab′ fragments; (iii) F (ab′) 2 fragments; (iv) Fd fragments; (v) Fv fragments; (vi) single-chain Fv (scFv) molecules; (vii) dAb fragments; and (viii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, cameliid antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), adnectins, small modular immunopharmaceuticals (SMIPs), and shark variable IgNAR domains, are also encompassed within the expression “antigen-binding fragment,” as used herein.

An antigen-binding fragment of an antibody will typically comprise at least one variable domain (e.g., at least one of a VH or VL). The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH—CH1-CH2; (V) VH—CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. In some embodiments, the hinge region comprises a glycine-serine linker.

Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art.

In certain embodiments of the disclosure, the anti-PAR2 antibodies of the disclosure are human antibodies. The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs, and in some embodiments, CDR3. However, the term “human antibody”, as used herein, is not intended to include antibodies in which CDR sequences derived from the germline of another mammalian species, such as a mouse, have been grafted onto human framework sequences.

The antibodies of the disclosure may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The current disclosure contemplates antibodies having one or more mutations in the hinge, CH2 or CH3 region which may be desirable, for example, in production, to improve the yield of the desired antibody form.

The antibodies of the disclosure may be isolated antibodies or isolated antigen-binding fragments. An “isolated antibody” or “isolated antigen-binding fragment,” as used herein, means an antibody or antigen-binding fragment that has been identified and separated and/or recovered from at least one component of its natural environment. For example, an antibody or antigen-binding fragment that has been separated or removed from at least one component of an organism, or from a tissue or cell in which the antibody naturally exists or is naturally produced, is an “isolated antibody” or an “isolated antigen-binding fragment” for purposes of the present disclosure. An isolated antibody also includes an antibody in situ within a recombinant cell. Isolated antibodies or antigen-binding fragments are antibodies or antigen-binding fragments that have been subjected to at least one purification or isolation step. According to certain embodiments, an isolated antibody or antigen-binding fragment may be substantially free of other cellular material and/or chemicals.

The anti-PAR2 antibodies or antigen-binding fragments disclosed herein may comprise one or more amino acid substitutions, insertions and/or deletions in the framework and/or CDR regions of the heavy and light chain variable domains as compared to the corresponding germline sequences from which the antibodies were derived. The present disclosure includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody or antigen-binding fragment was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). In some embodiments, the VH framework region 1 comprises an amino acid sequence that is at least 80%, 85%, 90%, 91%, 92%, 93%, 94%, 95%, 96%, 97%, 98%, 99% or 100% identical to the sequence of SEQ ID NO: 803. In some embodiments, the VH framework region 2 comprises an amino acid sequence that is at least 80%, 85%, 90%, 91%, 92%, 93%, 94%, 95%, 96%, 97%, 98%, 99% or 100% identical to the sequence of SEQ ID NO: 804. In some embodiments, the VH framework region 3 comprises an amino acid sequence that is at least 80%, 85%, 90%, 91%, 92%, 93%, 94%, 95%, 96%, 97%, 98%, 99% or 100% identical to the sequence of SEQ ID NO: 805. In some embodiments, the VH framework region 4 comprises an amino acid sequence that is at least 80%, 85%, 90%, 91%, 92%, 93%, 94%, 95%, 96%, 97%, 98%, 99% or 100% identical to the sequence of SEQ ID NO: 806. In some embodiments, the VL framework region 1 comprises an amino acid sequence that is at least 80%, 85%, 90%, 91%, 92%, 93%, 94%, 95%, 96%, 97%, 98%, 99% or 100% identical to the sequence of SEQ ID NO: 807. In some embodiments, the VL framework region 2 comprises an amino acid sequence that is at least 80%, 85%, 90%, 91%, 92%, 93%, 94%, 95%, 96%, 97%, 98%, 99% or 100% identical to the sequence of SEQ ID NO: 808. In some embodiments, the VL framework region 3 comprises an amino acid sequence that is at least 80%, 85%, 90%, 91%, 92%, 93%, 94%, 95%, 96%, 97%, 98%, 99% or 100% identical to the sequence of SEQ ID NO: 809. In some embodiments, the VL framework region 4 comprises an amino acid sequence that is at least 80%, 85%, 90%, 91%, 92%, 93%, 94%, 95%, 96%, 97%, 98%, 99% or 100% identical to the sequence of SEQ ID NO: 810. In some embodiments, the VH framework comprises 1, 2, 3, 4, 5, 6, 7, 8, 9 or 10 conservative substitutions as compared to a reference sequence of any one of SEQ ID NOs: 803-806. In some embodiments, the VL framework comprises 1, 2, 3, 4, 5, 6, 7, 8, 9 or 10 conservative substitutions as compared to a reference sequence of any one of SEQ ID NOs: 807-810.

Furthermore, the antibodies of the present disclosure may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present disclosure.

The present disclosure also includes anti-PAR2 antibodies comprising variants of any of the VH, VL, and/or CDR amino acid sequences disclosed herein having one or more conservative substitutions. For example, the present disclosure includes anti-PAR2 antibodies having VH, VL, and/or CDR amino acid sequences with, e.g., 10, 9, 8, 7, 6, 5, 4, 3, 2, or 1 conservative amino acid substitutions relative to any of the VH, VL, and/or CDR amino acid sequences disclosed herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

It should be noted that any portion of any of the antibodies or antigen-binding fragments of the disclosure may be similarly modified, such as with an epitope tag, a PEG moiety or moieties, and the like. Moreover, the antibodies or antigen-binding fragments may comprise more than one epitope tags, such as 2 epitope tags, or may include 0 epitope tags.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402. In some embodiments, the sequences are compared using EMBOSS Needle pairwise sequence alignment.

In some embodiments, the antibody or antigen-binding fragment is an antigen-binding fragment. In some embodiments, the antigen-binding fragment is an scFv. In some embodiments, the antigen-binding fragment is a Fab′. In some embodiments, the antibody or antigen-binding fragment is an antibody. In some embodiments, the antibody is a monoclonal antibody.

Antibodies became useful and of interest as pharmaceutical agents with the development of monoclonal antibodies. Monoclonal antibodies are produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63). In many cases, hybridomas are used to generate an initial antibody of murine or rodent origin. That initial antibody may then be modified, such as using recombinant techniques to produce rodent variants, chimeric antibodies, humanized antibodies and the like. Other methods exist to produce an initial antibody, and such methods are known in the art. However, regardless of the method used to generate an initial antibody or even a variant of that initial antibody, any given antibody of non-human origin can then be modified to increase its humanness.

Antibodies or antigen-binding fragments of the disclosure can be made by using combinatorial libraries to screen for antibodies with the desired activity or activities. For example, a variety of methods are known in the art for generating phage display libraries and screening such libraries for antibodies possessing the desired binding characteristics. Such methods are described generally in Hoogenboom et al. in Methods in Molecular Biology 178:1-37 (O'Brien et al., ed., Human Press, Totowa, NJ, 2001). For example, one method of generating antibodies of interest is through the use of a phage antibody library as described in Lee et al., J. Mol. Biol. (2004), 340 (5): 1073-93.

In principle, synthetic antibody clones are selected by screening phage libraries containing phage that display various fragments of antibody variable region (Fv) fused to phage coat protein. Such phage libraries are panned by affinity chromatography against the desired antigen. Clones expressing Fv fragments capable of binding to the desired antigen are adsorbed to the antigen and thus separated from the non-binding clones in the library. The binding clones are then eluted from the antigen, and can be further enriched by additional cycles of antigen adsorption/elution. Any of the antibodies of the disclosure can be obtained by designing a suitable antigen screening procedure to select for the phage clone of interest followed by construction of a full length antibody clone using the Fv sequences from the phage clone of interest and suitable constant region (Fc) sequences described in Kabat et al., Sequences of Proteins of Immunological Interest, Fifth Edition, NIH Publication 91-3242, Bethesda MD (1991), vols. 1-3. It can be advantageous to increase the humanness of a non-human antibody to make it more suitable for use in human subject and cells, whether for diagnostic, therapeutic, or research purposes. Antibodies may be modified for use as therapeutics. Examples of such antibodies (including antibody fragments) include chimeric, humanized, and fully human antibodies. Numerous methods exist in the art for the generation of chimeric, humanized and human antibodies. In the context of the present disclosure, an antibody is considered humanized if at least one of the VH domain or VL domain is humanized. Moreover, a VH or VL domain is humanized if the amino acid sequence of at least a portion of at least one of the FR regions has been modified, relative to a parent non-human (e.g., murine) antibody, such that the amino acid sequence of that portion corresponds to that of a human antibody or a human consensus sequence. In certain embodiments, at least one, two, three, or four FR regions of the VH domain and/or at least one, two, three, or four FR regions of the VL domain have been modified (in whole or in part) so that their sequence is more closely related to a human sequence. For any of the foregoing in certain embodiments, a humanized antibody fragment may be provided in the context of a human or non-human light chain and/or heavy chain constant region (e.g., comprising a CL and one or more of a CH1, hinge, CH2, and/or CH3 domains). In certain embodiments, a humanized antibody or antigen binding fragment of the disclosure is provided in the context of human light and/or heavy chain constant domains, when present. Antibodies and antibody binding fragments combining any of the humanized light chain variable domains and/or heavy chain variable domains described herein are exemplary of antibodies and antigen binding fragments of the disclosure. In some embodiments, the antibody or antigen-binding fragment is humanized. In some embodiments, the antibody or antigen-binding fragment is chimeric. In some embodiments, the antibody or antigen-binding fragment is human.

According to certain embodiments of the present disclosure, anti-PAR2 antibodies are provided comprising an Fc domain comprising one or more mutations which enhance or diminish antibody binding to the FcRn receptor, e.g., at acidic pH as compared to neutral pH. For example, the present disclosure includes anti-PAR2 antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). In yet another embodiment, the modification comprises a 265A (e.g., D265A) and/or a 297A (e.g., D297A) modification. All possible combinations of the foregoing Fc domain mutations, and other mutations within the antibody variable domains disclosed herein, are contemplated within the scope of the present disclosure. In some embodiments, antibodies comprise the triple mutation L234F/L235E/P331S (“TM”). TM causes a profound decrease in the binding activity of human IgG₁ molecules to human Clq, CD64, CD32A and CD16. See, e.g., Oganesyan et al., Acta Crystallogr D Biol Crystallogr. 64:700-704 (2008). Antibodies with increased half-lives may also be generated by modifying amino acid residues identified as involved in the interaction between the Fc and the FcRn receptor. For example, the introduction of the triple mutation M252Y/S254T/T256E (‘YTE’) into the CH2 domain of human immunoglobulin G (IgG) molecules causes an increase in their binding to the human neonatal Fc receptor (FcRn). See U.S. Pat. No. 7,083,784, the contents of which are herein incorporated by reference in its entirety. In some embodiments, the antibodies comprise the YTE modifications.

According to certain embodiments of the present disclosure, anti-PAR2 antibodies arc provided comprising one or more mutations in the VH and/or VL domains which enhance or diminish antibody binding to PAR2, e.g., at acidic pH as compared to neutral pH. For example, the present disclosure includes anti-PAR2 antibodies comprising a mutation in the CDR2 (SEQ ID NO: 4) or a CDR3 (SEQ ID NO: 5) region of the VH domain and/or the CDR3 (SEQ ID NO: 10) of the VL domain, wherein the mutation(s) replace one or more amino acids with histidine and decreases the affinity of the VH and/or VL domain to PAR2 in an acidic environment (e.g., in an endosome where pH ranges from about 5.5 to about 6.0). Such mutations may result in an increase in serum half-life of the antibody when administered to an animal. Non-limiting examples of such VH modifications include, e.g., a modification at amino acid positions 4, 5, 7, 8, 10, 11, 12, 14, 15, 16, and 17 of CDR2 (SEQ ID NO: 4) and 1, 2, 4, 5, and 7 of CDR3 (SEQ ID NO: 5). Non-limiting examples of such VL modifications include, e.g., a modification at positions 1, 2, 4, 5, 6, 7, 8, 9, 12, and 14 of CDR3 (SEQ ID NO: 10). In yet another embodiment, the VH comprises modifications at positions 5, 8, 12, 16, and 17 of CDR2 (SEQ ID NO: 4) and positions 2 and 3 of CDR3 (SEQ ID NO: 5). All possible combinations of the foregoing VH and VL domain mutations, and other mutations within the Fc domain disclosed herein, are contemplated within the scope of the present disclosure.

In some embodiments, the disclosure provides for an antibody or antigen-binding fragment thereof comprising a heavy chain variable domain (VH) and a light chain variable domain (VL), wherein the VH comprises: i) a VH-CDR1 having the amino acid sequence of SEQ ID NO: 3; ii) a VH-CDR2 having the amino acid sequence of SEQ ID NO: 4, but wherein a histidine is optionally present at any one or more of the amino acid positions corresponding to positions 1-17 (e.g., 4, 5, and 7-17) of SEQ ID NO: 4; and iii) a VH-CDR3 having the amino acid sequence of SEQ ID NO: 5, but wherein a histidine is optionally present at any one or more of the amino acid positions corresponding to positions 1-8 of SEQ ID NO: 5; and wherein the VL comprises: i) a VL-CDR1 having the amino acid sequence of SEQ ID NO: 8; ii) a VL-CDR2 having the amino acid sequence of SEQ ID NO: 9; and iii) a VL-CDR3 having the amino acid sequence of SEQ ID NO: 10; but wherein a histidine is optionally present at any one or more of the amino acid positions corresponding to positions 1-14 of SEQ ID NO: 10. In some embodiments, the VH comprises: i) a VH-CDR1 having the amino acid sequence of SEQ ID NO: 3, ii) a VH-CDR2 having the amino acid sequence of SEQ ID NO: 4, iii) a VH-CDR3 having the amino acid sequence of SEQ ID NO: 5, and wherein the VL comprises: i) a VL-CDR 1 having the amino acid sequence of SEQ ID NO: 8, ii) a VL-CDR2 having the amino acid sequence of SEQ ID NO: 9, iii) a VL-CDR3 having the amino acid sequence of SEQ ID NO: 10. In some embodiments, the antibody or antigen fragment thereof has a histidine at the amino acid position corresponding to any one or more of positions 7, 8, 12, 15, 16, or 17 of SEQ ID NO: 4. In some embodiments, the antibody or antigen fragment thereof has a histidine at the amino acid position corresponding to any one or more of positions 2 or 3 of SEQ ID NO: 5. In some embodiments, the antibody or antigen fragment thereof has a histidine at the amino acid position corresponding to any one or more of positions 1, 5, 6, or 14 of SEQ ID NO: 10. In some embodiments, a histidine is present at the amino acid positions corresponding to positions 5, 8, 12, 16, and 17 of SEQ ID NO: 4; and wherein a histidine is present at the amino acid positions corresponding to positions 2 and 3 of SEQ ID NO: 5. In some embodiments, the VH-CDR2 comprises an amino acid sequence selected from the group consisting of: SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714, 724, 734, 744, 754, 764, 774, 784, 794, and 811-818. In some embodiments, the VH-CDR3 comprises an amino acid sequence selected from the group consisting of: SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, and 819-820. In some embodiments, the VL-CDR3 comprises an amino acid sequence selected from the group consisting of: SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790 and 800. In some embodiments, the VH-CDR2 comprises an amino acid sequence corresponding to SEQ ID NO: 14; wherein the VH-CDR3 comprises an amino acid sequence corresponding to SEQ ID NO: 15, and wherein the VL-CDR3 comprises an amino acid sequence corresponding to SEQ ID NO: 20. In some embodiments, the VH-CDR2 comprises an amino acid sequence corresponding to SEQ ID NO: 811; the VH-CDR3 comprises an amino acid sequence corresponding to SEQ ID NO: 819, and the VL-CDR3 comprises an amino acid sequence corresponding to SEQ ID NO: 10 or 20. In some embodiments, the VH-CDR2 comprises an amino acid sequence corresponding to SEQ ID NO: 814; the VH-CDR3 comprises an amino acid sequence corresponding to SEQ ID NO: 820, and the VL-CDR3 comprises an amino acid sequence corresponding to SEQ ID NO: 10 or 20. In some embodiments, the VH-CDR2 comprises an amino acid sequence corresponding to SEQ ID NO: 816; the VH-CDR3 comprises an amino acid sequence corresponding to SEQ ID NO: 15, and the VL-CDR3 comprises an amino acid sequence corresponding to SEQ ID NO: 10 or 20. In some embodiments, the VH-CDR2 comprises an amino acid sequence corresponding to SEQ ID NO: 818; the VH-CDR3 comprises an amino acid sequence corresponding to SEQ ID NO: 15, and the VL-CDR3 comprises an amino acid sequence corresponding to SEQ ID NO: 10 or 20. In some embodiments, the VH comprises framework regions that are each at least 80%, 85%, 90%, 91%, 92%, 93%, 94%, 95%, 96%, 97%, 98%, 99% or 100% identical to SEQ ID NOs: 803-806. In some embodiments, the VL comprises framework regions that are each at least 80%, 85%, 90%, 91%, 92%, 93%, 94%, 95%, 96%, 97%, 98%, 99% or 100% identical to SEQ ID NOs: 807-810. In some embodiments, the VH comprises an amino acid sequence that is at least 80%, 85%, 90%, 91%, 92%, 93%, 94%, 95%, 96%, 97%, 98%, 99% or 100% identical to any of the sequences selected from the group consisting of: SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, and 792. In some embodiments, the VL comprises an amino acid sequence that is at least 80%, 85%, 90%, 91%, 92%, 93%, 94%, 95%, 96%, 97%, 98%, 99% or 100% identical to any of the sequences selected from the group consisting of: SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797. In some embodiments, the VH comprises an amino acid sequence corresponding to SEQ ID NO: 12 and the VL comprises an amino acid sequence corresponding to SEQ ID NO: 17. In some embodiments, the VH comprises an amino acid sequence corresponding to SEQ ID NO: 821 and the VL comprises an amino acid sequence corresponding to SEQ ID NO: 7 or 17. In some embodiments, the VH comprises an amino acid sequence corresponding to SEQ ID NO: 824 and the VL comprises an amino acid sequence corresponding to SEQ ID NO: 7 or 17. In some embodiments, the VH comprises an amino acid sequence corresponding to SEQ ID NO: 827 and the VL comprises an amino acid sequence corresponding to SEQ ID NO: 7 or 17. In some embodiments, the VH comprises an amino acid sequence corresponding to SEQ ID NO: 831 and the VL comprises an amino acid sequence corresponding to SEQ ID NO: 7 or 17.

The present disclosure also includes anti-PAR2 antibodies comprising a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG₁, human IgG₂ or human IgG₄ molecule, combined with part or all of a CH3 domain derived from a human IgG₁, human IgG₂ or human IgG₄ molecule. According to certain embodiments, the antibodies of the disclosure comprise a chimeric CH region having a chimeric hinge region. For example, a chimeric hinge may comprise an “upper hinge” amino acid sequence (amino acid residues from positions 216 to 227 according to EU numbering) derived from a human IgG₁, a human IgG₂ or a human IgG₄ hinge region, combined with a “lower hinge” sequence (amino acid residues from positions 228 to 236 according to EU numbering) derived from a human IgG₁, a human IgG₂ or a human IgG₄ hinge region.

According to certain embodiments, the chimeric hinge region comprises amino acid residues derived from a human IgG₁ or a human IgG₄ upper hinge and amino acid residues derived from a human IgG₂ lower hinge. An antibody comprising a chimeric CH region as described herein may, in certain embodiments, exhibit modified Fc effector functions without adversely affecting the therapeutic or pharmacokinetic properties of the antibody. (See, e.g., US 2015-0203591 A1).

The present disclosure includes anti-PAR2 antibodies or antigen-binding fragments which interact with one or more amino acids of PAR2. The epitope to which the antibodies bind may consist of a single contiguous sequence of 3 or more (e.g., 3, 4, 5, 6, 7, 8, 9, 10, 11, 12, 13, 14, 15, 16, 17, 18, 19, 20 or more) amino acids of PAR2. Alternatively, the epitope may consist of a plurality of non-contiguous amino acids (or amino acid sequences) of PAR2.

Various techniques known to persons of ordinary skill in the art can be used to determine whether an antibody “interacts with one or more amino acids” within a polypeptide or protein. Exemplary techniques include, e.g., routine cross-blocking assay such as that described Antibodies, Harlow and Lane (Cold Spring Harbor Press, Cold Spring Harb., NY), alanine scanning mutational analysis, peptide blots analysis (Reineke, 2004, Methods Mol Biol 248:443-463), and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267 (2): 252-259; Engen and Smith (2001) Anal. Chem. 73: 256A-265A.

The present disclosure further includes anti-PAR2 antibodies or antigen-binding fragments thereof that bind to the same epitope as any of the antibodies or antigen-binding fragments described herein (e.g., an antibody or antigen-binding fragment comprising the amino acid sequences of SEQ ID NOs: 12 and 17). Likewise, the present disclosure also includes anti-PAR2 antibodies and antigen-binding fragments that compete for binding to PAR2 with any of the antibodies or antigen-binding fragments described herein (e.g., an antibody or antigen-binding fragment comprising the amino acid sequences of SEQ ID NOs: 12 and 17). The skilled worker can easily determine whether an antibody binds to the same epitope as, or competes for binding with, a reference anti-PAR2 antibody by using routine methods known in the art and exemplified herein. For example, to determine if a test antibody binds to the same epitope as a reference anti-PAR2 antibody of the disclosure, the reference antibody is allowed to bind to a PAR2 protein. Next, the ability of a test antibody to bind to the PAR2 molecule is assessed. If the test antibody is able to bind to PAR2 following saturation binding with the reference anti-PAR2 antibody, it can be concluded that the test antibody binds to a different epitope than the reference anti-PAR2 antibody. On the other hand, if the test antibody is not able to bind to the PAR2 molecule following saturation binding with the reference anti-PAR2 antibody, then the test antibody may bind to the same epitope as the epitope bound by the reference anti-PAR2 antibody of the disclosure. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present disclosure, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502).

Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-PAR2 antibody, the above-described binding methodology is performed in two orientations: In a first orientation, the reference antibody is allowed to bind to a PAR2 protein under saturating conditions followed by assessment of binding of the test antibody to the PAR2 molecule. In a second orientation, the test antibody is allowed to bind to a PAR2 molecule under saturating conditions followed by assessment of binding of the reference antibody to the PAR2 molecule. If, in both orientations, only the first (saturating) antibody is capable of binding to the PAR2 molecule, then it is concluded that the test antibody and the reference antibody compete for binding to PAR2. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

Methods for generating monoclonal antibodies, including fully human monoclonal antibodies are known in the art. Any such known methods can be used in the context of the present disclosure to make human antibodies that specifically bind to human PAR2.

Using VELOCIMMUNE™ technology, for example, or any other known method for generating fully human monoclonal antibodies, high affinity chimeric antibodies to PAR2 are initially isolated having a human variable region and a mouse constant region. As in the experimental section below, the antibodies are characterized and selected for desirable characteristics, including affinity, selectivity, epitope, etc. If necessary, mouse constant regions are replaced with a desired human constant region, for example wild-type or modified IgG1 or IgG4, to generate a fully human anti-PAR2 antibody. While the constant region selected may vary according to specific use, high affinity antigen-binding and target specificity characteristics reside in the variable region. In certain instances, fully human anti-PAR2 antibodies are isolated directly from antigen-positive B cells.

The anti-PAR2 antibodies and antibody fragments of the present disclosure encompass proteins having amino acid sequences that may vary from those of the described antibodies but that retain the ability to bind PAR2 (e.g., SEQ ID NO: 801), or more specifically in some embodiments, a PAR2 tethered ligand (e.g., SEQ ID NO: 802). Such variant antibodies and antibody fragments comprise one or more additions, deletions, or substitutions of amino acids when compared to a parent sequence, but exhibit biological activity that is essentially equivalent to that of the described antibodies. Likewise, the anti-PAR2 antibody-encoding DNA sequences of the present disclosure encompass sequences that comprise one or more additions, deletions, or substitutions of nucleotides when compared to the disclosed sequences, but that encode an anti-PAR2 antibody or antibody fragment that is essentially bioequivalent to an anti-PAR2 antibody or antibody fragment of the disclosure. Examples of such variant amino acid and DNA sequences are discussed above.

Two antibodies or antigen-binding fragments are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies or antigen-binding fragments will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In some embodiments, two antibodies or antigen-binding fragments are bioequivalent if there are no clinically meaningful differences in their safety, purity, and potency.

In some embodiments, two antibodies or antigen-binding fragments are bioequivalent if a patient can be switched one or more times between the reference product and the biological product without an expected increase in the risk of adverse effects, including a clinically significant change in immunogenicity, or diminished effectiveness, as compared to continued therapy without such switching.

In some embodiments, two antibodies or antigen-binding fragments are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-PAR2 antibodies of the disclosure may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies or antigen-binding fragments may include anti-PAR2 antibody variants comprising amino acid changes which modify the glycosylation characteristics of the antibodies or antigen-binding fragments, e.g., mutations which eliminate or remove glycosylation.

The present disclosure, according to certain embodiments, provides anti-PAR2 antibodies or antigen-binding fragments that bind to human PAR2 but not to PAR2 from other species. The present disclosure also includes anti-PAR2 antibodies that bind to human PAR2 and to PAR2 from one or more non-human species. For example, the anti-PAR2 antibodies of the disclosure may bind to human PAR2 and may bind or not bind, as the case may be, to one or more of mouse, rat, guinea pig, hamster, gerbil, pig, cat, dog, rabbit, goat, sheep, cow, horse, camel, cynomolgus monkey, marmoset, rhesus or chimpanzee PAR2. According to certain embodiments, the antibodies or antigen-binding fragments bind to PAR2 in human A549 cells, rat KNRK cells, cynomolgus monkey CYNOM-K1 cells or mouse LL/2 cells.

The disclosure encompasses anti-PAR2 monoclonal antibodies conjugated to a therapeutic moiety (“immunoconjugate”), such as a cytotoxin, a chemotherapeutic, an immunosuppressant or a radioisotope. Examples of suitable cytotoxic agents and chemotherapeutic agents for forming immunoconjugates are known in the art (see for example, WO 05/103081).

In some embodiments, the antibodies of the present disclosure may be monospecific, bi-specific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer ci a/., 2004, Trends Biotechnol. 22:238-244. The anti-PAR2 antibodies or antigen-binding fragments of the present disclosure can be linked to or co-expressed with another functional molecule, e.g., another peptide or protein. For example, an antibody or antigen-binding fragment thereof can be functionally linked (e.g., by chemical coupling, genetic fusion, noncovalent association or otherwise) to one or more other molecular entities, such as another antibody or antigen-binding fragment to produce a bi-specific or a multispecific antibody with a second binding specificity. For example, the present disclosure includes bi-specific antibodies wherein one arm of an immunoglobulin is specific for human PAR2 or a fragment thereof, and the other arm of the immunoglobulin is specific for a second therapeutic target or is conjugated to a therapeutic moiety.

An exemplary bi-specific antibody or antigen-binding fragment format that can be used in the context of the present disclosure involves the use of a first immunoglobulin (Ig) CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V821 (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K_{D}, E79Q, and V821 (by IMGT; Q355R, N384S, K392N, V397M, R409K_{D}, E419Q, and V422I by EU) in the case of IgG4 antibodies. Variations on the bi-specific antibody format described above are contemplated within the scope of the present disclosure.

Other exemplary bispecific formats that can be used in the context of the present disclosure include, without limitation, e.g., scFv-based or diabody bispecific formats, IgG-scFv fusions, dual variable domain (DVD)-lg, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab<2>bispecific formats (see, e.g., Klein et al. 2012, mAbs 4:6, 1-1 1, and references cited therein, for a review of the foregoing formats). Bispecific antibodies or antigen-binding fragments can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. C em. Soc. [Epub: Dec. 4, 2012]).

### C. Nucleic Acids and Expression Systems

In some embodiments, the disclosure provides for a nucleic acid capable of expressing any of the antibodies of antigen-binding fragments disclosed herein. The nucleic acids may be single-stranded or double-stranded, DNA or RNA molecules. In further embodiments, the antibody or antigen-binding fragment nucleic acid sequences can be isolated, recombinant, and/or fused with a heterologous nucleotide sequence, or in a DNA library. In some embodiments, the nucleic acid comprises a nucleotide sequence that is at least 80%, 85%, 90%, 91%, 92%, 93%, 94%, 95%, 96%, 97%, 98%, 99% or 100% identical to any one of SEQ ID NOs: 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, 361, 371, 381, 391, 401, 411, 421, 431, 441, 451, 461, 471, 481, 491, 501, 511, 521, 531, 541, 551, 561, 571, 581, 591, 601, 611, 621, 631, 641, 651, 661, 671, 681, 691, 701, 711, 721, 731, 741, 751, 761, 771, 781, and/or 791. In some embodiments, the nucleic acid comprises a nucleotide sequence that is at least 80%, 85%, 90%, 91%, 92%, 93%, 94%, 95%, 96%, 97%, 98%, 99% or 100% identical to any one of SEQ ID NOs: 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, 366, 376, 386, 396, 406, 416, 426, 436, 446, 456, 466, 476, 486, 496, 506, 516, 526, 536, 546, 556, 566, 576, 586, 596, 606, 616, 626, 636, 646, 656, 666, 676, 686, 696, 706, 716, 726, 736, 746, 756, 766, 776, 786, and/or 796. In particular embodiments, the nucleic acid comprises a nucleotide sequence that is at least 80%, 85%, 90%, 91%, 92%, 93%, 94%, 95%, 96%, 97%, 98%, 99% or 100% identical to SEQ ID NO: 1, 6, 11 and/or 16

In certain embodiments, nucleic acids encoding antibodies or antigen-binding fragments also include nucleotide sequences that hybridize under highly stringent conditions to a polynucleotide encoding any of the above-mentioned antibodies or antigen-binding fragments nucleotide sequence, or complement sequences thereof. In some embodiments, the nucleic acids hybridize under highly stringent conditions to a polynucleotide encoding an amino acid sequence that is at least 80%, 85%, 90%, 91%, 92%, 93%, 94%, 95%, 96%, 97%, 98%, 99% or 100% identical to any one of SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, and 792. In some embodiments, the nucleic acids hybridize under highly stringent conditions to a polynucleotide encoding an amino acid sequence that is at least 80%, 85%, 90%, 91%, 92%, 93%, 94%, 95%, 96%, 97%, 98%, 99% or 100% identical to any one of SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797. One of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids encoding the antibody or antigen-binding fragment thereof due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In some embodiments, the disclosure provides for a vector comprising any of the nucleic acids disclosed herein. In some embodiments, the disclosure provides for a host cell comprising any of the vectors disclosed herein.

Regardless of when an antibody of the disclosure is a full length antibody or an antigen binding fragment, antibodies and antigen binding fragments of the disclosure can be recombinantly expressed in cell lines. In these embodiments, sequences encoding particular antibodies or antigen binding fragments can be used for transformation of a suitable host cell, such as a mammalian host cell or yeast host cell. According to these embodiments, transformation can be achieved using any known method for introducing polynucleotides into a host cell, including, for example packaging the polynucleotide in a virus (or into a viral vector) and transducing a host cell with the virus (or vector) or by transfection procedures known in the art. Generally, the transformation procedure used may depend upon the host to be transformed. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

According to certain embodiments of the disclosure, a nucleic acid molecule encoding the amino acid sequence of a heavy chain constant region (all or a portion), a heavy chain variable region of the disclosure, a light chain constant region, or a light chain variable region of the disclosure is inserted into an appropriate expression vector using standard ligation techniques. In a preferred embodiment, the heavy or light chain constant region is appended to the C-terminus of the appropriate variable region and is ligated into an expression vector. The vector is typically selected to be functional in the particular host cell employed (i.e., the vector is compatible with the host cell machinery such that amplification of the gene and/or expression of the gene can occur). For a review of expression vectors, see, Goeddel (ed.), 1990, Meth. Enzymol. Vol. 185, Academic Press. N.Y. In the context of antibody expression, both the heavy and light chain may be expressed from the same vector (e.g., from the same or different promoters present on the same vector) or the heavy and light chains may be expressed from different vectors. In certain embodiments, the heavy and light chains are expressed from different vectors which are transfected into the same host cell and co-expressed. Regardless of when the heavy and light chains are expressed in the same host cell from the same or a different vector, the chains can then associate to form an antibody (or antibody fragment, depending on the portions of the heavy and light chain being expressed).

Typically, expression vectors used in any of the host cells will contain sequences for plasmid maintenance and for cloning and expression of exogenous nucleotide sequences. Such sequences, collectively referred to as “flanking sequences” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. These portions of vectors are well known, and there are numerous generally available vectors that can be selected and used for the expression of proteins. One can readily select vectors based on the desired host cell and application.

An origin of replication is typically a part of those prokaryotic expression vectors purchased commercially, and the origin aids in the amplification of the vector in a host cell. If the vector of choice does not contain an origin of replication site, one may be chemically synthesized based on a known sequence, and ligated into the vector. For example, the origin of replication from the plasmid pBR322 (New England Biolabs, Beverly, Mass.) is suitable for most gram-negative bacteria and various viral origins (e.g., SV40, polyoma, adenovirus, vesicular stomatitus virus (VSV), or papillomaviruses such as HPV or BPV) are useful for cloning vectors in mammalian cells. Generally, the origin of replication component is not needed for mammalian expression vectors (for example, the SV40 origin is often used only because it also contains the virus early promoter).

The expression and cloning vectors of the disclosure will typically contain a promoter that is recognized by the host organism and operably linked to the molecule encoding heavy and/or light chain. Promoters are untranscribed sequences located upstream (i.e., 5′) to the start codon of a structural gene (generally within about 100 to 1000 bp) that control the transcription of the structural gene. Promoters are conventionally grouped into one of two classes: inducible promoters and constitutive promoters. Inducible promoters initiate increased levels of transcription from DNA under their control in response to some change in culture conditions, such as the presence or absence of a nutrient or a change in temperature. Constitutive promoters, on the other hand, initiate continual gene product production; that is, there is little or no control over gene expression. A large number of promoters, recognized by a variety of potential host cells, are well known. A suitable promoter is operably linked to the DNA encoding the heavy chain or light chain comprising an antibody or antigen binding fragment of the disclosure. In certain embodiments, the same promoter is used for both the heavy and light chain. In other embodiments, different promoters (present on the same or different vectors) are used for each.

Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus and most preferably Simian Virus 40 (SV40). Other suitable mammalian promoters include heterologous mammalian promoters, for example, heat-shock promoters and the actin promoter.

Additional promoters which may be of interest include, but are not limited to: the SV40 early promoter region (Bernoist and Chambon, 1981, Nature 290:304-10); the CMV promoter; the promoter contained in the 3′ long terminal repeat of Rous sarcoma virus (Yamamoto et al., 1980, Cell 22:787-97); the herpes thymidine kinase promoter (Wagner et al., 1981, Proc. Natl. Acad. Sci. USA 78:1444-45); the regulatory sequences of the metallothionine gene (Brinster et al., 1982, Nature 296:39-42); prokaryotic expression vectors such as the beta-lactamase promoter (Villa-Kamaroff et al., 1978, Proc. Natl. Acad. Sci. USA 75:3727-31); or the tac promoter (DeBoer et al., 1983, Proc. Natl. Acad. Sci. USA 80:21-25). Also of interest are the following animal transcriptional control regions, which exhibit tissue specificity and have been utilized in transgenic animals: the elastase I gene control region that is active in pancreatic acinar cells (Swift et al., 1984, Cell 38:639-46; Ornitz et al., 1986, Cold Spring Harbor Symp. Quant. Biol. 50:399-409 (1986); MacDonald, 1987, Hepatology 7:425-515); the insulin gene control region that is active in pancreatic beta cells (Hanahan, 1985, Nature 315:115-22); the immunoglobulin gene control region that is active in lymphoid cells (Grosschedl et al., 1984, Cell 38:647-58; Adames et al., 1985, Nature 318:533-38; Alexander et al., 1987, Mol. Cell. Biol. 7:1436-44); the mouse mammary tumor virus control region that is active in testicular, breast, lymphoid and mast cells (Leder et al., 1986, Cell 45:485-95); the albumin gene control region that is active in liver (Pinkert et al., 1987, Genes and Devel. 1:268-76); the alpha-feto-protein gene control region that is active in liver (Krumlauf et al., 1985, Mol. Cell. Biol. 5:1639-48; Hammer et al., 1987, Science 235:53-58); the alpha 1-antitrypsin gene control region that is active in liver (Kelsey et al., 1987, Genes and Devel. 1:161-71); the beta-globin gene control region that is active in myeloid cells (Mogram et al., 1985, Nature 315:338-40; Kollias et al., 1986, Cell 46:89-94); the myelin basic protein gene control region that is active in oligodendrocyte cells in the brain (Readhead et al., 1987, Cell 48:703-12); the myosin light chain-2 gene control region that is active in skeletal muscle (Sani, 1985, Nature 314:283-86); and the gonadotropic releasing hormone gene control region that is active in the hypothalamus (Mason et al., 1986, Science 234:1372-78).

The vector may also include an enhancer sequence to increase transcription of DNA encoding light chain or heavy chain.

Expression vectors of the disclosure may be constructed from a starting vector such as a commercially available vector. Such vectors may or may not contain all of the desired flanking sequences. Where one or more of the flanking sequences described herein are not already present in the vector, they may be individually obtained and ligated into the vector. Methods used for obtaining each of the flanking sequences are well known to one skilled in the art.

After the vector has been constructed and a nucleic acid molecule encoding light chain or heavy chain or light chain and heavy chain comprising an antibody or antigen binding fragment of the disclosure has been inserted into the proper site of the vector, the completed vector may be inserted into a suitable host cell for amplification and/or polypeptide expression. The transformation of an expression vector into a selected host cell may be accomplished by well known methods including transfection, infection, calcium phosphate co-precipitation, electroporation, microinjection, lipofection, DEAE-dextran mediated transfection, or other known techniques. The method selected will in part be a function of the type of host cell to be used. These methods and other suitable methods are well known to the skilled worker.

The host cell, when cultured under appropriate conditions, synthesizes the antibody or antigen binding fragment of the disclosure that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and case of folding into a biologically active molecule.

Mammalian cell lines available as host cells for expression are well known in the art and include, but are not limited to, many immortalized cell lines available from the American Type Culture Collection (A.T.C.C.), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. In another embodiment, one may select a cell line from the B cell lineage that does not make its own antibody but has a capacity to make and secrete a heterologous antibody (e.g., mouse myeloma cell lines NSO and SP2/0). In other embodiments, a cell other than a mammalian cell is used, such as a yeast cell line (e.g., Pichia).

In certain embodiments, the cell line stably expresses an antibody or antigen binding fragment of the disclosure. In other embodiments, the cells transiently express an antibody or antigen binding fragment of the disclosure.

### D. Therapeutic Formulation and Administration

The disclosure provides pharmaceutical compositions comprising the anti-PAR2 antibodies or antigen-binding fragments thereof of the present disclosure. The pharmaceutical compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-31 1.

The dose of antibody administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering anti-PAR2 antibodies or antigen-binding fragments may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intrathecal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In some embodiments, the antibodies and antigen-binding fragments thereof have utility in treating conditions and disorders associated with the central nervous system, and, particularly associated with the brain. While various factors must be considered when administering a macromolecule such as an antibody or antigen-binding fragment to a subject's brain, i.e., ability of the antibody or antigen-binding fragment to cross the blood-brain-barrier (BBB), the skilled worker is aware of methods of administering such macromolecules to the brain. For example, in some embodiments, the antibody or antigen-binding fragment is covalently modified with one or more cationic polyamines, such as hexamethylenediamine or tetramethylenediamine in order to increase the likelihood that the antibody or antigen-binding fragment is internalized across the BBB. In some embodiments, the antibody or antigen-binding fragment is a bispecific antibody or antigen-binding fragment, wherein the antibody or fragment targets PAR2 and also targets a receptor that facilitates transport across the BBB (e.g., transferrin receptor, insulin receptor and TMEM30A). In some embodiments, the antibody or antigen-binding fragment is conjugated to an agent that targets a receptor that facilitates transport across the BBB (e.g., transferrin receptor, insulin receptor and TMEM30A). In some embodiments, the BBB is temporarily disrupted prior to or during administration of the antibody or fragment. In some embodiments the BBB is temporarily disrupted by means of ultrasound, radiation, biochemical treatment (e.g., with a Kca receptor agonist such as NS-1619), or intra-arterial infusion of concentrated hyperosmotic solutions.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25TM pen, HUMALOG™ pen, HUMALIN 70/30TM pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intrathecal, intracutaneous and intramuscular injections, drip infusions, etc. These injectable preparations may be prepared by methods publicly known. For example, the injectable preparations may be prepared, e.g., by dissolving, suspending or emulsifying any of the antibodies or antigen-binding fragments disclosed herein or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody or antigen-binding fragment is contained in about 5 to about 100 mg and in about 10 to about 250 mg for the other dosage forms.

### E. Therapeutic Uses of the Antibodies

For any of the methods described herein, the disclosure contemplates the use of any of the antibodies or antigen-binding fragments of the disclosure.

In some embodiments, the disclosure provides for a method of treating a disorder in a subject in which undesired and/or aberrant PAR2 activity is involved, comprising administering any of the antibodies or antigen-binding fragments described herein. As used herein, “disorder”, “condition” and “disease” are used interchangeably to refer to any of the disorders, conditions or diseases disclosed herein. In some embodiments, the disease/disorder/condition in which undesired and/or aberrant PAR2 activity is involved is a disease/disorder/condition associated with aberrant or undesired inflammation. Examples of diseases/disorders/conditions in which aberrant or undesired PAR2 activity is involved include acute or chronic pain, acute or chronic itch, acute or chronic inflammation (e.g., acute or chronic inflammation of the joints, lungs, brain, gastrointestinal tract, periodontium, skin, and vascular systems), autoimmune disorders, periodontitis, osteoarthritis, rheumatoid arthritis, inflammatory bowel disease, arthritis, psoriasis, obesity, diabetes, cardiovascular disease, pancreatitis, cancer (e.g., breast, lung, colon, stomach or prostate cancer), asthma, fibrosis, gastric ulcers, fibrosis or fibrotic disorders, Alzheimer's Disease, Parkinson's Disease, contract dermatitis, Crohn's Disease, ulcerative colitis, adult respiratory distress syndrome (ARDS), glomerulonephritis, and meningitis. In some embodiments, the disclosure provides for methods of treating a subject with metabolic syndrome, or one or more conditions associated with metabolic syndrome, such as visceral fat deposition, hypertension, impaired glucose and insulin homeostasis, insulin resistance, endothelial damage, cardiovascular hypertrophy, inflammation, vascular inflammation, atherosclerosis, ventricular contractile dysfunction, fibrosis and fatty liver disease. In particular embodiments, the disclosure provides for methods of treating pain, e.g., pain associated with any of the diseases/disorders/conditions disclosed herein (e.g., osteoarthritic pain). In some embodiments, the subject is a mammal. In some embodiments, the subject is a human.

In some embodiments, the disclosure provides for a method of interfering with the interaction between a protease (e.g., trypsin) and PAR2, comprising the step of administering any of the antibodies or antigen-binding fragments described herein to a cell. In some embodiments, the disclosure provides for a method of inhibiting exposure of the tethered ligand of PAR2 on a cell, comprising the step of administering any of the antibodies or antigen-binding fragments described herein to a cell. In some embodiments, the disclosure provides for a method of inhibiting the interaction between the tethered ligand of PAR2 and the second transmembrane loop of the PAR2 protein, comprising the step of administering any of the antibodies or antigen-binding fragments described herein to a cell. In some embodiments, the disclosure provides for a method of inhibiting activation of a PAR2 receptor on a cell, comprising the step of administering any of the antibodies or antigen-binding fragments described herein to a cell. In some embodiments, the cell is a neuron (e.g., a sensory neuron). In some embodiments, the cell is in vitro. In other embodiments, the cell is in a subject. In some embodiments, the subject is a mammal. In some embodiments, the subject is a human. In some embodiments, the subject suffers from any of disorders disclosed herein.

For any of the methods described herein, the disclosure contemplates the combination of any step or steps of one method with any step or steps from another method. These methods involve administering to an individual in need thereof an effective amount of a compound of the disclosure appropriate for the particular disease or condition. In specific embodiments, these methods involve delivering any of the antibodies or antigen-binding fragments disclosed herein to the cells of a subject in need thereof.

The terms “treatment”, “treating”, “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes any one or more of: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). For example, “treatment” of pain (e.g., osteoarthritic pain) involves a reduction, arrest, alleviation, or elimination of pain symptoms in the treated subject. The population of subjects treated by the method of the disease includes subjects suffering from the undesirable condition or disease, as well as subjects at risk for development of the condition or disease.

For any of the methods described herein, the disclosure contemplates the use of any of the antibodies or antigen-binding fragments described throughout the application. In addition, for any of the methods described herein, the disclosure contemplates the combination of any step or steps of one method with any step or steps from another method.

In certain embodiments, the present invention provides methods of treating conditions associated with any of the diseases/conditions/disorders disclosed herein, e.g., acute or chronic pain (e.g., osteoarthritic pain). These methods involve administering to the individual a therapeutically effective amount of any of the antibodies or antigen-binding fragments as described above. These methods are particularly aimed at therapeutic and prophylactic treatments of animals, and more particularly, humans. The disclosure contemplates all combinations of any of the foregoing aspects and embodiments, as well as combinations with any of the embodiments set forth in the detailed description and examples.

By the term “therapeutically effective dose” is meant a dose that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

In certain embodiments, any of the antibodies or antigen-binding fragments of the present invention can be administered alone or in combination with one or more additional compounds or therapies for treating any of the diseases/conditions/disorders disclosed herein, e.g., acute or chronic pain (e.g., osteoarthritic pain). For example, any of the antibodies or antigen-binding fragments disclosed herein can be co-administered in conjunction with one or more therapeutic compounds. When co-administration is indicated, the combination therapy may encompass simultaneous or alternating administration. In addition, the combination may encompass acute or chronic administration. Optionally, the antibody/antigen-binding fragment and additional compounds act in an additive or synergistic manner for treating any of the diseases/conditions/disorders disclosed herein, e.g., acute or chronic pain (e.g., osteoarthritic pain). Additional compounds to be used in combination therapies include, but are not limited to, small molecules, polypeptides, antibodies, antisense oligonucleotides, and siRNA molecules. In some embodiments, the additional compound is any one or more of: an anti-inflammatory drug, analgesic, a nonsteroidal anti-inflammatory drug (NSAID), corticosteroid, hyaluronic acid, acetaminophen, codeine, lorcet, lortab, vicodin, hydrocodone, morphine, oxycontin, Roxicodone, Percocet, aspirin, celecoxib, pregabalin, joint fusion, joint replacement, abatacept, adalimumab, anakinra, certolizumab, etanercept, golimumab, infliximab, rituximab, tocilizumab and tofacitinib. Depending on the nature of the combinatory therapy, administration of the antibodies or antigen binding disclosures of the disclosure may be continued while the other therapy is being administered and/or thereafter. Administration of the antibodies or antigen-binding fragments may be made in a single dose, or in multiple doses. In some instances, administration of the antibodies or antigen binding fragments is commenced at least several days prior to the other therapy, while in other instances, administration is begun either immediately before or at the time of the administration of the other therapy. In some embodiments, any of the additional compounds disclosed herein is conjugated to any of the antibodies or antigen-binding fragments disclosed herein.

In another example of combination therapy, any of the antibodies or antigen-binding fragments of the disclosure can be used as part of a therapeutic regimen combined with one or more additional treatment modalities. By way of example, such other treatment modalities include, but are not limited to, dietary therapy, occupational therapy, physical therapy, psychiatric therapy, massage, acupuncture, acupressure, mobility aids, assistance animals, and the like.

Note that although the antibodies or antigen-binding fragments described herein can be used in combination with other therapies, in certain embodiments, an antibody or antigen-binding fragment is provided as the sole form of therapy. Regardless of whether administrated alone or in combination with other medications or therapeutic regiments, the dosage, frequency, route of administration, and timing of administration of the antibodies or antigen-binding fragments is determined by a physician based on the condition and needs of the patient.

According to certain embodiments of the present disclosure, multiple doses of an anti-PAR2 antibody or antigen-binding fragment thereof (or a pharmaceutical composition comprising a combination of an anti-PAR2 antibody and any of the additional therapies mentioned herein) may be administered to a subject over a defined time course. The methods according to this aspect of the disclosure comprise sequentially administering to a subject multiple doses of an anti-PAR2 antibody or antigen-binding fragment of the disclosure. As used herein, “sequentially administering” means that each dose of anti-PAR2 antibody or antigen-binding fragment is administered to the subject at a different point in time, e.g., on different days separated by a predetermined interval (e.g., hours, days, weeks or months). The present disclosure includes methods which comprise sequentially administering to the patient a single initial dose of an anti-PAR2 antibody or antigen-binding fragment, followed by one or more secondary doses of the anti-PAR2 antibody or antigen-binding fragment, and optionally followed by one or more tertiary doses of the anti-PAR2 antibody or antigen-binding fragment.

The terms “initial dose,” “secondary doses,” and “tertiary doses,” refer to the temporal sequence of administration of the anti-PAR2 antibody or antigen-binding fragment of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-PAR2 antibody or antigen-binding fragment, but generally may differ from one another in terms of frequency of administration. In certain embodiments, however, the amount of anti-PAR2 antibody or antigen-binding fragment contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

### F. Diagnostic/Other Uses of the Antibodies or Antigen Binding Fragments

The anti-PAR2 antibodies of the present disclosure may also be used to detect and/or measure PAR2, or PAR2-expressing cells in a sample, e.g., for diagnostic purposes. For example, an anti-PAR2 antibody, or antigen-binding fragment thereof, may be used to diagnose a condition or disease characterized by aberrant expression (e.g., over-expression, under-expression, lack of expression, etc.) of PAR2. Exemplary diagnostic assays for PAR2 may comprise, e.g., contacting a sample obtained from a patient, with an anti-PAR2 antibody of the disclosure, wherein the anti-PAR2 antibody is labeled with a detectable label or reporter molecule.

Alternatively, an unlabeled anti-PAR2 antibody can be used in diagnostic applications in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure PAR2 in a sample include enzyme-linked immunosorbent assay (ELISA), radioimmunoassay (RIA), and fluorescence-activated cell sorting (FACS).

The compositions of the disclosure have numerous uses. For example, the antibodies and antigen binding fragments of the disclosure are useful for studying preferential cell and tissue distribution in cells and in tissues in vitro and/or in vivo. Similarly, the antibodies and antigen binding fragments, either alone or conjugated to a heterologous agent are useful as imaging agents, such as for ex vivo or in vivo diagnostic applications. For example, the antibodies or antigen binding fragments conjugated to a radioactive moiety are useful for ex vivo or in vivo imaging studies. Similarly, any of the antibodies or antigen binding fragments of the disclosure are similarly useful.

When used in vitro, the antibodies and antigen binding fragments of the disclosure are suitable for identifying binding partners for the antibody or antigen binding fragment being delivered (e.g., identifying proteins or peptides that bind the antibody or antigen binding fragment), and for evaluating localization and trafficking. Similarly, when used in vivo, the antibodies or antigen-binding fragments are useful for identifying binding partners for the antibody or antigen-binding fragment being delivered (e.g., identifying proteins or peptides that bind the antibody or antigen-binding fragment), for evaluating localization and trafficking, for evaluating biodistribution and half-life, and for evaluating immunogenicity.

### G. Animal/Cell Models

Numerous animal models are known to the skilled worker that would be useful for examining any of the antibodies or fragments thereof. See, e.g., Kuyinu et al., 2016, J Orthop Surg Res, 11 (19): 10.1186/s13018-016-0346-5. In some embodiments, the animal model is a pain-based model generated by treatment of the animal with a chemical, such as sodium monoiodoacetate (MIA) or carrageenan. In some embodiments, the chemical is injected at the site of where the pain is to be induced in the animal. In some embodiments, the animal model is an animal in which an injury is introduced postoperatively (e.g., incisional), such as anterior cruciate ligament transection, meniscectomy, or medial meniscal transection. In some embodiments, the animal model is one associated with an inflammatory condition, such as lower esophageal irritation, colon inflammation, stomach ulceration, urinary bladder inflammation, pancreatic inflammation and uterine inflammation. See, e.g., the animal models referred to in National Research Council Committee on Recognition and Alleviation of Pain in Laboratory Animals, “Models of Pain,” 2009.

### H. Kits

In certain embodiments, the invention also provides a pharmaceutical package or kit comprising one or more containers filled with at least one antibody or antigen-binding fragment of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Generation of Antibodies with pH Sensitive Binding for PAR2

### Generation of Recombinant Human, Rat and Cynomolgus PAR2 and PAR1 Proteins.

The human, rat and cynomolgus (Macaca fascicularis) PAR2 (Proteinase-Activated Receptor 2) constructs comprising extracellular residues 1-75 were designed with N-terminal AviTag™ (Avidity LLC) and C-terminal Flag and poly Histidine tags and cloned into the vector pDEST12.2 OriP FH (Life Technologies). The human PAR1 (Proteinase-Activated Receptor 1) construct comprising extracellular residues 1-102 was designed with C-terminal Flag and poly Histidine tags and cloned into the vector pDEST12.2 OriP FH (Life Technologies). The constructs were expressed in HEK293 cells and purified from the media using standard affinity and size exclusion chromatography purification. To generate biotinylated proteins the AviTag™ was biotinylated enzymatically according to the manufacturer's instructions.

### Construction of Combinatorial Histidine Scanning Libraries

Split pool oligonucleotides were designed to introduce histidine or the wild type amino acid at each position of either the VHCDR2, VHCDR3 or VLCDR3 of Par0067. Three Par0067 scFv phage display libraries were subsequently constructed in which there were between 0% and 100% histidine residues in either the VHCDR2, VHCDR3 or VLCDR3.

Selection of pH Sensitive Par0067 Variant scFv's

The combinatorial histidine scanning libraries were subjected to affinity based phage display selections with the aim of isolating Par0067 variants which bind to PAR2 at pH 7.4 but with reduced binding at pH 6.0. To achieve this, four rounds of selection were performed with each library using decreasing concentrations of biotinylated recombinant human PAR2 (Hawkins, R E et al., 1992 Aug. 5; 226 (3): 889-96). At each round, phage were pre-incubated for 1 hour with streptavidin coated paramagnetic beads (Dynabeads®) to remove any streptavidin binders. The streptavidin beads were subsequently removed with a DYNAL® magnet and discarded and the remaining phage added to biotinylated recombinant human PAR2 at pH 7.4. The selection proceeded for 2 hours before adding streptavidin coated paramagnetic beads to capture the biotinylated recombinant human PAR2 bound phage. The beads were washed 5-times with PBS Tween (PBST) prior to elution of specific scFv in low pH buffer (pH 5.5-pH 6.0). The selected scFv-phage particles were then rescued as described previously (Osbourn J K. et al. Immunotechnology, 2 (3): 181-96, 1996), and the selection process was repeated in the presence of decreasing concentrations of biotinylated PAR2 (1 nM-0.05 nM over 4 rounds).

Reformatting of scFv to IgG1-TM

Antibodies were converted from scFv to whole immunoglobulin G1 triple mutant (IgG₁-TM, IgG₁ Fc sequence incorporating mutations L234F, L235E and P331S) antibody format essentially as described by Persic et al. (1997, Gene, 187, 9-18) with the following modifications. An OriP fragment was included in the expression vectors to facilitate use with CHO-transient cells and to allow episomal replication. The variable heavy (VH) domain was cloned into a vector containing the human heavy chain constant domains and regulatory elements to express whole IgG1-TM heavy chain in mammalian cells. Similarly, the variable light (VL) domain was cloned into a vector for the expression of the human light chain (lambda) constant domains and regulatory elements to express whole IgG light chain in mammalian cells. To obtain IgGs, the heavy and light chain IgG expressing vectors were transfected into CHO-transient mammalian cells (Daramola et al. Biotechnol Prog 30 (1): 132-41 (2014)). IgGs were expressed and secreted into the medium. Harvests were filtered prior to purification, then IgG was purified using Protein A chromatography. Culture supernatants were loaded on a column of appropriate size of Ceramic Protein A (BioSepra) and washed with 50 mM Tris-HCl pH 8.0, 250 mM NaCl. Bound IgG was eluted from the column using 0.1 M Sodium Citrate (pH 3.0) and neutralized by the addition of Tris-HCl (pH 9.0). The eluted material was buffer exchanged into PBS using Nap 10 columns (Amersham, #17-0854-02) and the concentration of IgG was determined spectrophotometrically using an extinction coefficient based on the amino acid sequence of the IgG (Mach et al., Anal. Biochem. 200 (1): 74-80 (1992)). The purified IgG were analyzed for aggregation and degradation purity using SEC-HPLC and by SDS-PAGE.

Screening for pH Sensitive Par0067 Variant scFv's and IgG's

In order to screen for and profile antibodies with potential pH dependent binding a biochemical epitope competition assay format was used. The assay, which used Homogeneous Time Resolved Fluorescence (HTRF™) technology, was designed to evaluate the ability of test antibodies (scFv or IgG) to inhibit the interaction of the parent Par0067 IgG antibody binding to the human PAR2 extracellular domain (ECD). Importantly the assay was implemented at two different pH values (pH 7.4 and pH 6.0).

Parallel assays at pH 7.4 and pH 6.0 (as described above) were first used to screen crude un-purified scFv (bacterial extracts) in a single point 384 well high throughput screen (HTS). This single point parallel HTS format enabled the screening of many 1000's of test scFv and those antibodies which resulted in reduced inhibition of the interaction of parent Par0067 IgG binding to human PAR2 at pH 6.0 versus pH 7.4 were taken forward for further characterisation. Subsequently the same epitope competition assay approach was then implemented in a multipoint dose response IC₅₀ format to test both purified scFv and purified IgG (in the latter case a minor modification of the assay design was required as set out in Section C). Again assays were implemented at pH 7.4 and pH 6.0 however in these experiments we were most interested in test antibodies where dose response inhibition curves showed a large rightward shift (i.e. significantly increased IC₅₀ value) at pH 6.0 relative to corresponding dose response inhibition curves observed at pH 7.4.

The following protocol includes methods for both single point testing of crude un-purified scFv and subsequent testing of purified scFv and IgG.

### Section A: Generic Assay Conditions

### Assay Buffers

Assay buffers were made fresh on the day of use. For experiments at pH 7.4 DPBS (Gibco 14190-086) was supplemented with KF (0.4M) (VWR, 103444T) and BSA (0.1% w/v) (PAA, K05-013) followed by rechecking the pH and fine adjusting to pH 7.4 as required. For experiments at pH 6.0, while keeping all other buffer components identical with the pH 7.4 assay buffer outlined above, the pH 6.0 assay buffer was formulated using a base buffer of 200 mM MES (Sigma, M-5287) (as opposed to DPBS). After addition of KF (0.4 M) and BSA (0.1% w/v) the 200 mM MES based buffer was then adjusted to pH 6.0 with HCL.

Assay Plates: Assays were performed using black shallow well 384 plates (Round bottom, non-binding) (Corning, 4514)

Assay Volume: 20 μl

Incubation and Plate Reading: Assay plates were incubated for 2 hrs at RT before reading using a standard HTRF™ read protocol on an Envision plate reader.

Section B: Testing of scFv: (Un-Purified Bacterial Lysates and Purified scFv)

Addition Order and Assay Components:

| | Total | nsb | Test |
|---|---|---|---|
| Par0067 IgG (×4 [final]) | 5 μl | 5 μl | 5 μl |
| Test scFv (×4 [final]) | — | — | 5 μl |
| Assay Buffer | 5 μl | 5 μl | — |
| Bio-human-PAR2 ECD (×4 [final]) | 5 μl | — | 5 μl |
| Assay Buffer | — | 5 μl | — |
| Europium Cryptate Labelled Streptavidin plus XL⁶⁶⁵ | 5 μl | 5 μl | 5 μl |
| labelled anti-human-Fc (both ×4 [final]) | | | |

Par0067 IgG Preparation/Addition:

Unlabelled purified Par0067 IgG (generated in house) was made up to a concentration of 4.44 nM (1.11 nM final [assay]) in each of the two assay buffers previously described in Section A (pH 7.4 & pH 6.0). 5 μl/well of the appropriate pH 4.44 nM Par0067 IgG solution was added to all wells of the relevant assay plate (pH 7.4 and pH 6.0).

Test scFv Preparation/Addition:
a) For parallel single point HTS of crude un-purified bacterial lysate scFv samples at both pH 7.4 and pH 6.0 samples were first pre-diluted to 40% of their neat concentration using either pH 7.4 or pH 6.0 assay buffer as appropriate. Subsequently 5 μl of 40% pre-diluted sample was then transferred into the appropriate assay (pH 7.4 or pH 6.0) in order to give a final assay sample concentration of 10.0% (in the 20 μl final assay volume). The parent Par0067 scFv was included in all HTS experiments as a control as were specific pH dependent antibodies as those became available (for benchmarking).
b) For multipoint dose response IC50 testing of purified scFv antibody, samples were tested from a top final assay of ¼ of the neat undiluted sample (i.e. no pre-dilution step was performed). Duplicate 11 point 1:3 serial dilutions then were prepared on 384 well polypropylene Greiner plates in each of the two assay buffers (pH 7.4 & 6.0). 5 μl per well of each serial dilution was transferred from the relevant pH scFv dilution plate to the corresponding assay plate (pH 7.4 and pH 6.0). 5 μl of the appropriate pH assay buffer was added to the total and non-specific wells. The parent Par0067 purified scFv was included in all multipoint dose response IC50 experiments as a control as were specific pH dependent purified scFv as those became available (for benchmarking). Data are presented in Table 1.

Biotinylated Human PAR2 ECD Preparation/Addition:

In house generated biotinylated human PAR2 ECD was diluted into each of the two assay buffers (pH 7.4 & pH 6.0) to give working solutions at 4.0 nM (1 nM final assay concentration). μl/well of the appropriate 4.0 nM biotinylated human PAR2 ECD working solution was then added to all wells of the corresponding assay plate (pH 7.4 and pH 6.0) with the exception of the negative binding control wells. 5 μl/well of the appropriate pH assay buffer was added to the negative binding wells.

HTRF Detection Reagent Preparation/Addition:

Europium Cryptate Labelled Streptavidin (CisBio, 610SAKLB) and XL⁶⁶⁵ Labelled Anti-Human-Fc (CisBio, 61HFCXLB) were each diluted into each pH assay buffer (pH 7.4, & pH 6.0) to give combined working solutions at concentrations of 6.0 nM (Europium Cryptate Labelled Streptavidin) and 40 nM (XL⁶⁶⁵ Labelled Anti-Human-Fc). Allowing for the ×4 fold dilution into the assay this resulted in final assay concentrations of 1.5 nM (Europium Cryptate Labelled Streptavidin) and 10 nM (XL⁶⁶⁵ Labelled Anti-Human-Fc). 5 μl/well of the relevant pH combined HTRF™ detection reagent working solution was then added to all wells of the corresponding assay plate (pH 7.4 and pH 6.0).

### Section C: Testing of Purified IgG:

Addition Order and Assay Components:

| | Total | nsb | Test |
|---|---|---|---|
| Dylight⁶⁵⁰ labelled Par0067 IgG (×4 [final]) | 5 μl | 5 μl | 5 μl |
| Test IgG (×4 [final]) | — | — | 5 μl |
| Assay Buffer | 5 μl | 5 μl | — |
| Bio-human-PAR2 ECD (×4 [final]) | 5 μl | — | 5 μl |
| Assay Buffer | — | 5 μl | — |
| Europium Cryptate Labelled Streptavidin (×4 [final]) | 5 μl | 5 μl | 5 μl |

Par0067 IgG Dylight650 Labelling:

Dylight⁶⁵⁰ labelling of Par0067 IgG was performed using a Dylight⁶⁵⁰ labelling kit (Thermo Scientific Cat. No. 84536). Labelling of in-house generated purified Par0067 IgG was performed according to the manufacturer's recommended labelling procedure. The final Dylight⁶⁵⁰ labelled Par0067 IgG concentration was determined as 0.56 mg/ml with a mean Dylight⁶⁵⁰ to IgG dye incorporation ratio of 2.7 moles dye/mole IgG.

Dylight⁶⁵⁰ Labelled Par0067 IgG Preparation/Addition:

Dylight⁶⁵⁰ labelled Par0067 IgG (0.56 mg/ml, 3,733 nM) was made up to a concentration of 4.44 nM (to give 1.11 nM final [assay]) in each of the two assay buffers previously described in the materials section (pH 7.4 & pH 6.0). 5 μl/well of the appropriate pH 4.44 nM Dylight⁶⁵⁰ Par0067 IgG solution was added to all wells of the relevant assay plate (pH 7.4 and pH 6.0).

Test IgG Serial Dilution/Addition:

Parent Par0067 IgG (used as a reference/control in all assays) was pre-diluted to give a 2000 nM stock in each of the two different pH assay buffers (in order to give a top final assay IgG concentration of 500 nM). All test, or other reference/control IgG were tested from a top final assay concentration of ¼ of the neat undiluted sample (i.e. no pre-dilution step was performed). Duplicate 11 point 1:3 serial dilutions then were prepared on 384 well polypropylene Greiner plates in each of the two assay buffers (pH 7.4 & 6.0). 5 μl per well was transferred from the relevant pH IgG dilution plate to the corresponding assay plate (pH 7.4 and pH 6.0). 5 μl of the appropriate pH assay buffer was added to the total and non-specific wells.

Biotinylated Human PAR2 ECD Preparation/Addition:

In house generated biotinylated human PAR2 ECD was diluted into each of the two assay buffers (pH 7.4 & pH 6.0) to give working solutions at 4.0 nM (1 nM final assay concentration). μl/well of the appropriate 4.0 nM biotinylated human PAR2 ECD working solution was then added to all wells of the corresponding assay plate (pH 7.4 and pH 6.0) with the exception of the negative binding control wells. 5 μl/well of the appropriate pH assay buffer was added to the negative binding wells.

HTRF Detection Reagent Preparation/Addition:

Europium Cryptate Labelled Streptavidin (CisBio, 610SAKLB) was diluted into each pH assay buffer (pH 7.4, & pH 6.0) to give work solutions at a concentration of 6.0 nM. Allowing for the x4 fold dilution into the assay this resulted in a final assay Europium Cryptate Labelled Streptavidin concentration of 1.5 nM. 5 μl/well of the relevant pH Europium Cryptate Labelled Streptavidin working solution was then added to all wells of the corresponding assay plate (pH 7.4 and pH 6.0).

Section D: Data Analysis:

665 nm and 620 nm counts were first converted to 665/620 ratio values. Delta F (%) was then calculated according to the following equation:

$$Delta F ( \% ) = ( ( sample ratio - negative ratio ) / negative ratio ) * 100$$

Negative ratio values were calculated in the absence of Par0067 IgG from the relevant negative binding control wells.

% Specific Binding was then calculated according to the following equation:

$$\% Specific Binding = \{ ( Sample \% Delta F - Negative Binding \% Delta F ) / ( Total Binding \% Delta F - Negative Binding \% Delta F ) \} * 100$$

For single point HTS % Specific Binding at pH 6.0 versus pH 7.4 was plotted on x and y axes, respectively, in order to visualise the distribution of scFv with reduced inhibition (higher % Specific Binding) at pH 6.0 relative to pH 7.4.

For multipoint dose response curves % Specific Binding values were plotted versus test purified antibody concentration (scFv or IgG). IC₅₀ values were determined via a sigmoidal dose response inhibition variable slope curve fit (4 parameter logistic equation) using Graphpad Prism Software.

TABLE 1

| | Par0067 Epitope Competition Assay | | | |
|---|---|---|---|---|
| | | | Variant: | Fold IC₅₀ |
| | pH 7.4 | pH 6.0 | Fold IC₅₀ | pH 7.4 |
| | IC₅₀ | IC₅₀ | (pH 6.0 v | (Variant v |
| Clone (scFv) | (nM) | (nM) | pH 7.4) | Par0067) |
| Par0067 | 3.3 | 3.6 | 1.1 | 1.0 |
| PaB670010 | 15.4 | 41.8 | 2.7 | 4.7 |
| PaB670020 | 5.6 | 18.5 | 3.3 | 1.7 |
| PaB670034 | 5.6 | 14.2 | 2.5 | 1.7 |
| PaB670045 | 9.8 | 72.5 | 7.4 | 3.0 |
| PaB670048 | 14.5 | 139.4 | 9.6 | 4.4 |
| PaB670064 | 67.1 | 289.9 | 4.3 | 20.3 |
| PaB670066 | 52.2 | 486.9 | 9.3 | 15.8 |
| PaB670067 | 14.4 | 113.2 | 7.9 | 4.4 |
| PaB670068 | 77.8 | 447.6 | 5.8 | 23.6 |
| PaB670070 | 60.4 | 270.0 | 4.5 | 18.3 |
| PaB670071 | 101.2 | 424.8 | 4.2 | 30.7 |
| PaB670073 | 72.1 | 428.3 | 5.9 | 21.8 |
| PaB670075 | 101.8 | 603.0 | 5.9 | 30.8 |
| PaB670076 | 46.2 | 291.8 | 6.3 | 14.0 |
| PaB670077 | 55.4 | 619.9 | 11.2 | 16.8 |
| PaB670078 | 33.3 | 319.2 | 9.6 | 10.1 |
| PaB670079 | 44.2 | 489.8 | 11.1 | 13.4 |
| PaB670080 | 16.6 | 158.1 | 9.5 | 5.0 |
| PaB670081 | 51.4 | 260.0 | 5.1 | 15.6 |
| PaB670082 | 25.4 | 77.9 | 3.1 | 7.7 |
| PaB670083 | 45.2 | 151.8 | 3.4 | 13.7 |
| PaB670084 | 54.5 | 282.7 | 5.2 | 16.5 |
| PaB670085 | 48.1 | 154.7 | 3.2 | 14.6 |
| PaB670087 | 40.9 | 130.7 | 3.2 | 12.4 |
| PaB670088 | 29.8 | 114.7 | 3.8 | 9.0 |
| PaB670089 | 29.9 | 134.0 | 4.5 | 9.1 |
| PaB670090 | 22.6 | 85.5 | 3.8 | 6.8 |
| PaB670091 | 25.2 | 98.9 | 3.9 | 7.6 |
| PaB670092 | 41.1 | 193.5 | 4.7 | 12.5 |
| PaB670093 | 18.6 | 58.7 | 3.2 | 5.6 |
| PaB670094 | 72.5 | 236.8 | 3.3 | 22.0 |
| PaB670095 | 20.0 | 113.2 | 5.7 | 6.1 |
| PaB670097 | 21.8 | 77.9 | 3.6 | 6.6 |
| PaB670098 | 65.9 | 210.3 | 3.2 | 20.0 |
| PaB670099 | 12.9 | 62.4 | 4.8 | 3.9 |
| PaB670100 | 20.3 | 69.0 | 3.4 | 6.2 |
| PaB670101 | 146.9 | 496.5 | 3.4 | 44.5 |
| PaB670102 | 5.3 | 13.7 | 2.6 | 1.6 |
| PaB670103 | 27.9 | 203.5 | 7.3 | 8.5 |
| PaB670104 | 21.7 | 202.2 | 9.3 | 6.6 |
| PaB670105 | 74.4 | 495.5 | 6.7 | 22.5 |
| PaB670106 | 312.8 | 1188.0 | 3.8 | 94.8 |
| PaB670107 | 42.7 | 463.9 | 10.9 | 12.9 |
| PaB670108 | 29.1 | 148.6 | 5.1 | 8.8 |

Recombination of Multiple Histidines into a Single scFv

Histidine residues from scFv which demonstrated pH dependent binding in the Par0067 epitope competition binding assay were recombined using site directed mutagenesis (Reikofski J and Tao B Y, 1992, Biotechnol Adv, 10 (4): 535-547). Resulting scFv which contained histidines in two different CDRs were retested in the competition binding assay as whole immunoglobulin G1 triple mutant (IgG1-TM) (Table 2) to identify variants with further improvements in pH dependent binding compared to the parent antibody, Par0067.

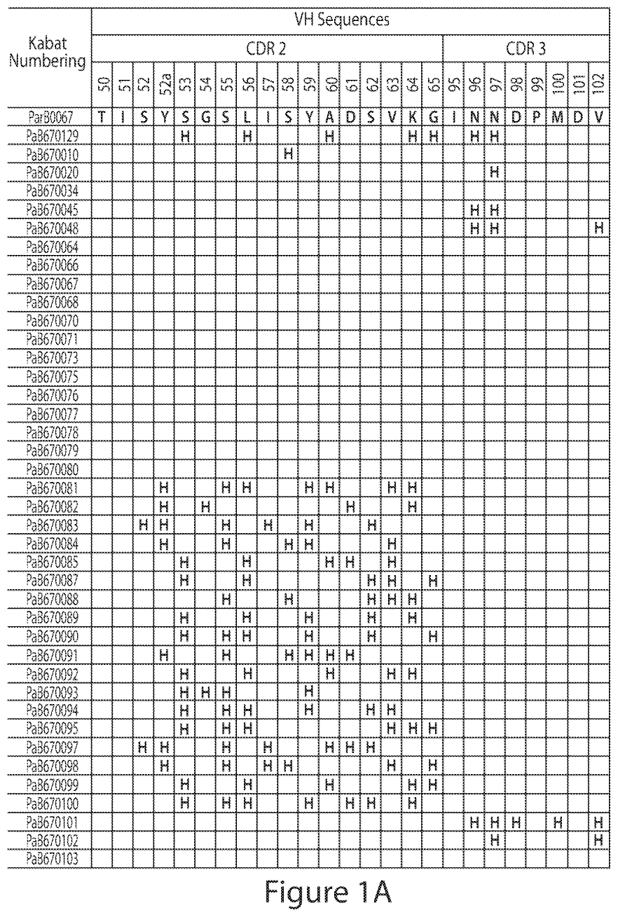

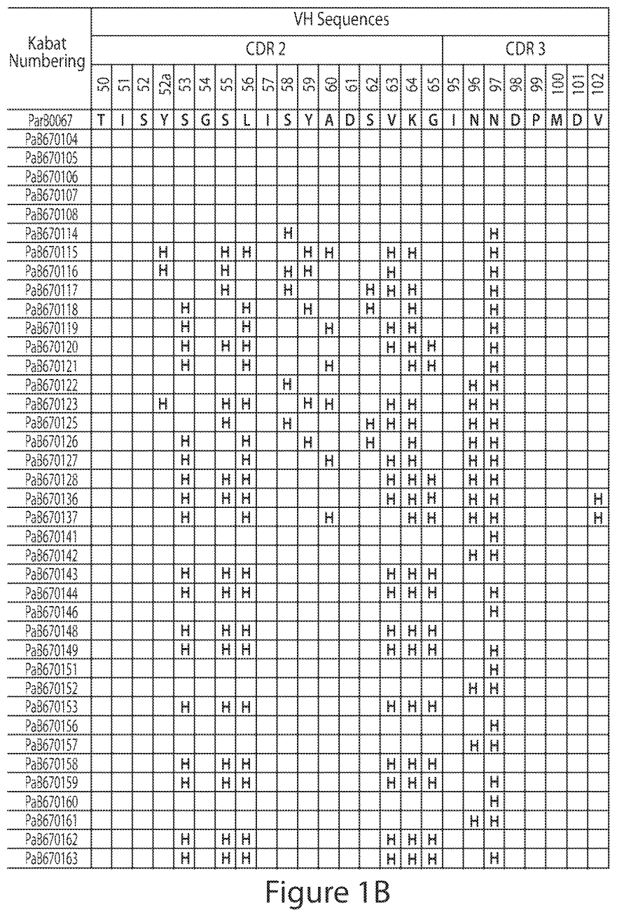

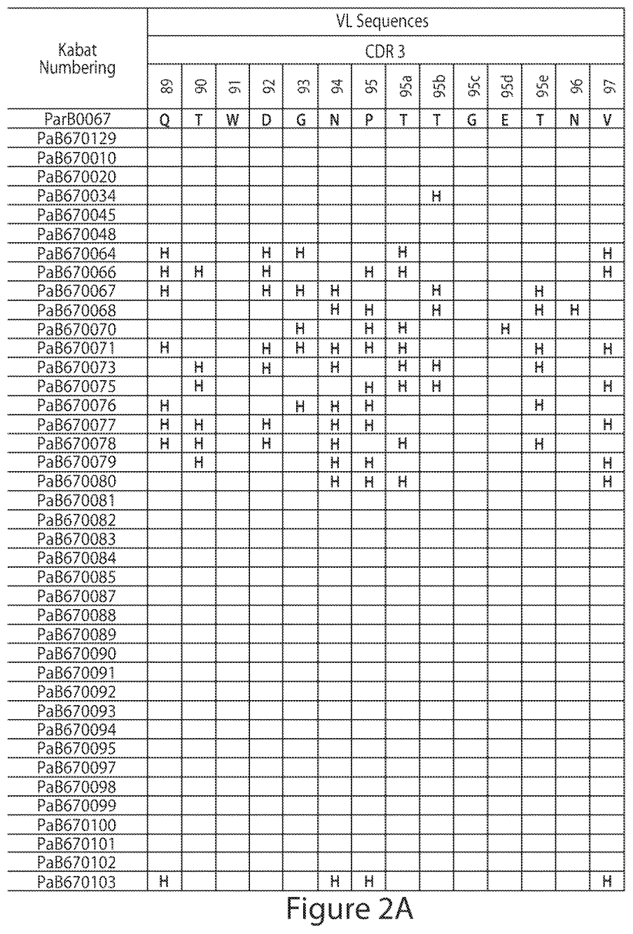

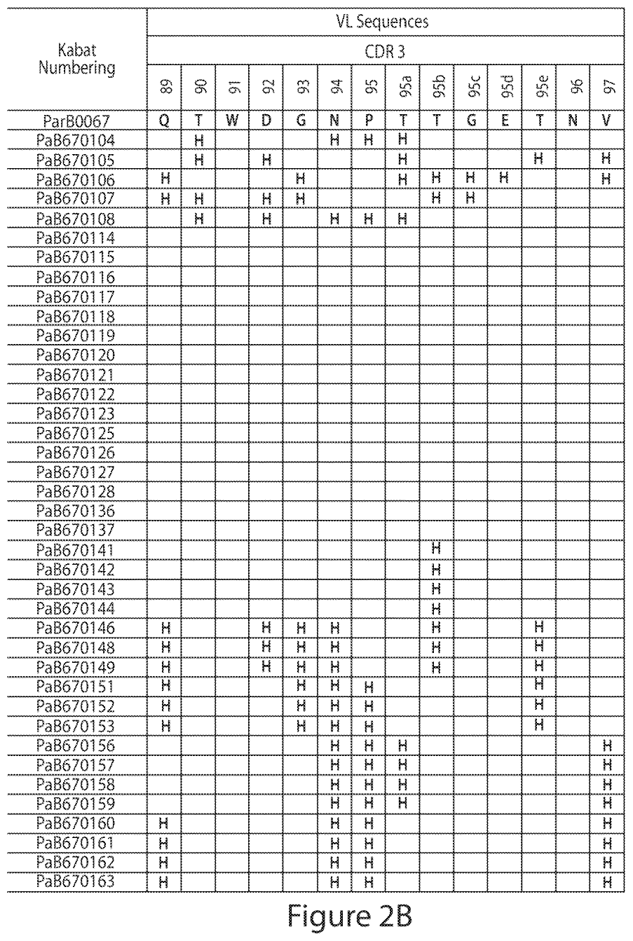

Sequence alignments for each of the histidine modified clones as compared to the reference Par0067 antibody CDR sequences are provided in FIGS. 1A-1B and 2A-2B.

TABLE 2

| | Par0067 Epitope Competition Assay | | | |
|---|---|---|---|---|
| | | | Variant: | Fold IC₅₀ |
| | pH 7.4 | pH 6.0 | Fold IC₅₀ | pH 7.4 |
| | IC₅₀ | IC₅₀ | (pH 6.0 v | (Variant v |
| Clone (IgG) | (nM) | (nM) | pH 7.4) | Par0067) |
| Par0067 | 0.835 | 0.790 | 0.95 | 1.00 |
| PaB670045 | 2.9 | 25.8 | 8.9 | 3.5 |
| PaB670048 | 5.7 | 48.2 | 8.5 | 6.8 |
| PaB670128 | 32.8 | IC | ND | 39.3 |
| PaB670129 | 56.1 | IC | ND | 67.2 |
| PaB670084 | 10.3 | 64.6 | 6.3 | 12.3 |
| PaB670141 | 2.4 | 13.9 | 5.9 | 2.8 |
| PaB670142 | 4.8 | 67.1 | 14.0 | 5.7 |
| PaB670143 | 3.4 | 40.0 | 11.8 | 4.0 |
| PaB670144 | 29.0 | 564.8 | 19.5 | 34.7 |
| PaB670146 | 91.9 | 784.3 | 8.5 | 110.1 |
| PaB670148 | 48.5 | 844.6 | 17.4 | 58.1 |
| PaB670149 | 504.9 | IC | ND | 604.7 |
| PaB670151 | 69.5 | 796.0 | 11.5 | 83.2 |
| PaB670152 | 438.8 | 6604.0 | 15.1 | 525.5 |
| PaB670153 | 93.0 | 1639.0 | 17.6 | 111.4 |
| PaB670156 | 7.0 | 190.0 | 27.2 | 8.4 |
| PaB670157 | 15.8 | 932.4 | 59.0 | 18.9 |
| PaB670158 | 6.8 | 477.0 | 69.8 | 8.2 |
| PaB670159 | 161.0 | IC | ND | 192.8 |
| PaB670160 | 16.9 | 634.3 | 37.5 | 20.2 |
| PaB670161 | 47.9 | 1899.0 | 39.6 | 57.4 |
| PaB670162 | 11.4 | 906.2 | 79.5 | 13.7 |
| PaB670163 | 145.1 | IC | ND | 173.8 |

IC = Incomplete Curve;
ND = Not Demonstrated

### Affinity of Anti-PAR2 Fabs for Human, Rat and Cynomolgus PAR2 as Determined by BIACORE™

Antigen binding fragments (Fabs) of the anti-PAR2 antibodies were expressed (Spooner J. et al. (2015) Biotechnol Bioeng. 112:1472-7) and the affinity for recombinant PAR2 of various species (human, rat and cynomolgus) determined by Biacore.

### Biacore Affinity Analysis

The affinity of the anti-PAR2 Fabs was measured using the Biacore T100 at 25° C. at various pH's (pH 7.4, pH 6.0 and pH 5.6). The experiments were carried out using recombinant human, rat and cynomolgus PAR2 with an N-terminal Avi tag and C-terminal Flag-His tags.

Streptavidin was covalently immobilised to a Cl chip surface using standard amine coupling techniques at a concentration of 4 μg/ml in 10 mM Sodium acetate pH 4.5. A final streptavidin surface of approximately 30-100 RUs was achieved. Recombinant biotinylated PAR2 species (produced in-house) were titrated onto the streptavidin chip surface at 4 μg/ml in HBS-EP+ buffer to enable Fab binding at saturation (Rₘₐₓ). This low level of analyte binding ensured minimal mass transport effects.

The anti-PAR2 Fabs were serially diluted (0.39 nM-25 nM) in HBS-EP+ buffer pH 7.4 or MES-BS-EP+ pH 6.0 buffer or in MES-BS-EP+ pH 5.6 buffer and flowed over the chip at 50 μl/min, with 3 minutes association and up to 30 minutes dissociation. Multiple buffer-only injections were made under the same conditions to allow for double reference subtraction of the final sensorgram sets, which were analysed using the BiaEval software (version 2.0.1). The chip surface was fully regenerated with pulses of 4 M MgCl₂.

BiaCore affinity results for select clones are provided below in Tables 3-13.

TABLE 3
Par0067 Fab.

| pH | Species | kₐ (M⁻¹ s⁻¹) | k_{d} (s⁻¹) | K_{D} (pM) | Rmax |
|---|---|---|---|---|---|
| 7.4 | Human | 6.66E+6 | 4.96E−5 | 7.5 | 50.1 |
| 6.0 | Human | 3.12E+6 | 9.72E−5 | 31.2 | 39.3 |
| 7.4 | Rat | 5.16E+6 | 1.94E−4 | 37.6 | 111 |
| 6.0 | Rat | 3.21E+6 | 5.44E−4 | 170 | 100 |

TABLE 4
PaB670048 Fab.

| pH | Species | kₐ (M⁻¹ s⁻¹) | k_{d} (s⁻¹) | K_{D} (pM) | Rmax |
|---|---|---|---|---|---|
| 7.4 | Human | 4.20E+6 | 5.19E−4 | 124 | 69.0 |
| 6.0 | Human | 1.94E+6 | 4.97E−3 | 2569 | 58.3 |
| 7.4 | Rat | 5.24E+6 | 3.07E−3 | 586 | 124.7 |
| 6.0 | Rat | 4.00E+6 | 2.05E−2 | 5120 | 105.2 |

TABLE 5
PaB670084 Fab.

| pH | Species | kₐ (M⁻¹ s⁻¹) | k_{d} (s⁻¹) | K_{D} (pM) | Rmax |
|---|---|---|---|---|---|
| 7.4 | Human | 6.49E+6 | 1.62E−3 | 250 | 68.4 |
| 6.0 | Human | 1.73E+6 | 7.63E−3 | 4,410 | 104.6 |
| 7.4 | Rat | 6.83E+6 | 8.37E−3 | 1226 | 97.6 |
| 16.0 | Rat | 2.57E+6 | 2.77E−2 | 10,800 | 91.97 |

TABLE 6
PaB670076 Fab.

| pH | Species | kₐ (M⁻¹ s⁻¹) | k_{d} (s⁻¹) | K_{D} (pM) | Rmax |
|---|---|---|---|---|---|
| 7.4 | Human | 5.84E+6 | 1.21E−3 | 207 | 63.5 |
| 6.0 | Human | 1.11E+6 | 5.14E−3 | 4,611 | 99.51 |
| 7.4 | Rat | 6.34E+6 | 1.07E−2 | 1,689 | 87.9 |
| 6.0 | Rat | 1.66E+6 | 5.10E−2 | 30,730 | 82.61 |

TABLE 7
PaB670120 Fab.

| pH | Species | kₐ (M⁻¹ s⁻¹) | k_{d} (s⁻¹) | K_{D} (pM) | Rmax |
|---|---|---|---|---|---|
| 7.4 | Human | 5.54E+6 | 1.37E−3 | 247 | 61.62 |
| 6.0 | Human | 3.48E+6 | 2.10E−2 | 6,047 | 94.67 |
| 7.4 | Rat | 5.62E+6 | 1.46E−2 | 2,606 | 84.23 |
| 6.0 | Rat | 8.26E+6 | 0.281 | 34,040 | 73.35 |

TABLE 8
PaB670128 Fab.

| pH | Species | kₐ (M⁻¹ s⁻¹) | k_{d} (s⁻¹) | K_{D} (pM) | Rmax |
|---|---|---|---|---|---|
| 7.4 | Human | 5.95E+6 | 3.65E−3 | 613 | 59.57 |
| 6.0 | Human | 2.22E+6 | 4.57E−2 | 20,062 | 79.4 |
| 7.4 | Rat | 6.49E+6 | 1.96E−2 | 3,023 | 80.86 |
| 6.0 | Rat | 1.79E+6 | 6.89E−2 | 38,520 | 63.26 |

TABLE 9
PaB670048 Fab.

| pH | Species | kₐ (M⁻¹ s⁻¹) | k_{d} (s⁻¹) | K_{D} (pM) | Rmax |
|---|---|---|---|---|---|
| 7.4 | Human | 5.59E+6 | 5.80E−4 | 104 | 58.53 |
| 6.0 | Human | 1.84E+6 | 4.58E−3 | 2,488 | 93.02 |
| 7.4 | Rat | 7.46E+6 | 3.25E−3 | 435 | 81.2 |
| 6.0 | Rat | 4.84E+6 | 2.17E−2 | 4,483 | 80.14 |

TABLE 10
PaB670129 Fab.

| pH | Species | kₐ (M⁻¹ s⁻¹) | k_{d} (s⁻¹) | K_{D} (pM) | Rmax |
|---|---|---|---|---|---|
| 7.4 | Human | 8.20E+6 | 5.04E−3 | 614 | 54.98 |
| 6.0 | Human | 1.59E+6 | 5.54E−2 | 34,840 | 76.02 |
| 7.4 | Rat | 8.82E+6 | 2.63E−2 | 2,979 | 73.16 |
| 6.0 | Rat | 1.45E+6 | 8.41E−2 | 57,910 | 67.27 |

TABLE 11
PaB670136 Fab.

| pH | Species | kₐ (M⁻¹ s⁻¹) | k_{d} (s⁻¹) | K_{D} (pM) | Rmax |
|---|---|---|---|---|---|
| 7.4 | Human | 5.17E+6 | 4.95E−3 | 957 | 50.27 |
| 6.0 | Human | 7.92E+8 | 14.9 | 18,840 | 67.97 |
| 7.4 | Rat | 7.57E+6 | 3.14E−2 | 4,149 | 73.24 |
| 6.0 | Rat | 1.57E+6 | 5.89E−2 | 37,550 | 47.7 |

TABLE 12
PaB670103 Fab.

| pH | Species | kₐ (M⁻¹ s⁻¹) | k_{d} (s⁻¹) | K_{D} (pM) | Rmax |
|---|---|---|---|---|---|
| 7.4 | Human | 3.52E+6 | 1.11E−4 | 32.5 | 46.3 |
| 6.0 | Human | 3.36E+5 | 6.07E−4 | 1,805 | 70.66 |
| 7.4 | Rat | 3.04E+6 | 5.58E−4 | 184 | 74.2 |
| 6.0 | Rat | 5.31E+5 | 5.42E−3 | 10,200 | 69.58 |

TABLE 13
PaB670129 Fab at 3 different pH's.

| pH | Species | kₐ (M⁻¹ s⁻¹) | k_{d} (s⁻¹) | K_{D} (pM) | Rmax |
|---|---|---|---|---|---|
| 7.4 | Human | 7.14E+6 | 5.34E−3 | 747.8 | 75.9 |
| | | 6.81E+6 | 5.30E−3 | 778.0 | 66.5 |
| 6.0 | Human | 1.56E+6 | 5.76E−2 | 36,910 | 58.1 |
| | | 3.26E+6 | 0.109 | 33,500 | 48.4 |
| 5.6 | Human | 1.33E+6 | 0.112 | 84,550 | 42.5 |
| 7.4 | Cynomolgus | 6.67E+6 | 2.23E−3 | 333.3 | 59.7 |
| | | 6.59E+6 | 2.21E−3 | 335.0 | 51.9 |
| 6.0 | Cynomolgus | 2.14E+6 | 2.75E−2 | 12,840 | 49.6 |
| | | 1.74E+6 | 2.36E−2 | 13,560 | 44.2 |
| 5.6 | Cynomolgus | 3.66E+6 | 0.139 | 37,840 | 41.3 |

### Example 2: Cell-Based PAR2 and PAR1 Activity Assay

Human A549 cells, rat KNRK, mouse LL/2 or cynomolgus CYNOM-K1 cells expressing endogenous PAR2, or human 1321N1-hPAR2-c18 cells overexpressing human PAR2 were seeded at 5,000 (human, cyno) or 7,000 (mouse, rat) cells per well on PDL-coated Tissue Culture Plates, (Greiner Bio-One). Cells were loaded with Fluo-Screen Quest™ Fluo-8 No Wash Calcium dye (AAT Bioquest, Inc). Cells were pre-treated with IgGs or Fabs diluted in assay buffer (HBSS, 0.1% BSA, 20 mM HEPES) for 1 h at room temperature. For mouse and cyno assays, cells were pre-treated with 0.5 nM or 10 nM thrombin, respectively, to desensitize PAR1 activity. PAR2 calcium responses to 11 nM (human), 400 nM (mouse) or 80 nM (rat, cyno) trypsin (Polymun) were measured on a Fluorescent Imaging Plate Reader (FLIPR) Tetra (Molecular Devices). To determine functional activity against human PAR1, thrombin driven calcium responses in the A549 human cell line were determined also on the FLIPR tetra and in these assays neutralising anti-PAR1 IgGs WEDE15 (Beckman Coulter) and ATAP2 (Life Technologies) were used as positive controls. To determine functional activity against diverse proteases, 1321N1-hPAR2-c18 cells overexpressing human PAR2 were pre-treated with PaB670129, prior to stimulating calcium release with 0.5 nM trypsin, 500 nM tryptase or 1 nM matriptase. Fluorescence measurements were measured before, during, and after protease addition and peak RFU calculated per well. % responses (relative to protease alone) were calculated against antibody concentration and IC50s determined using GraphPad Prism software.

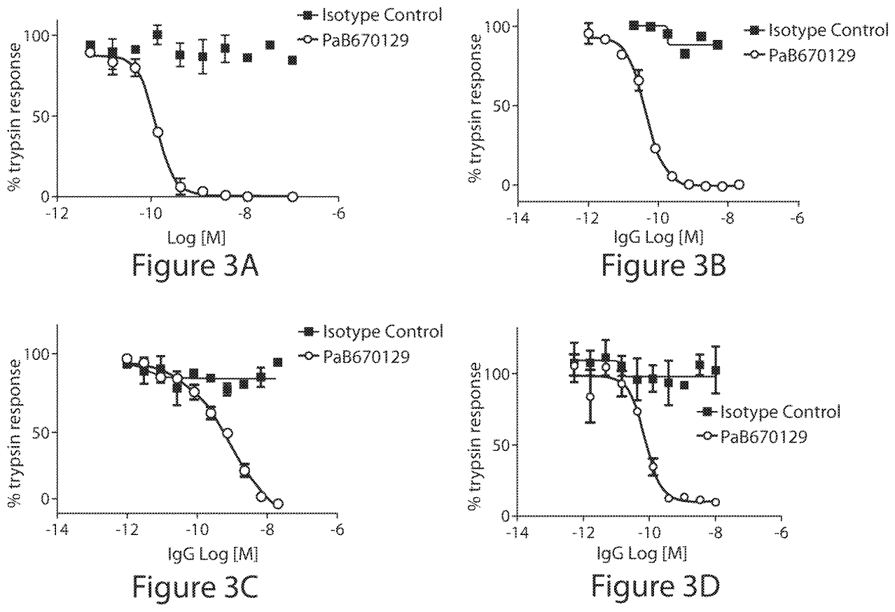

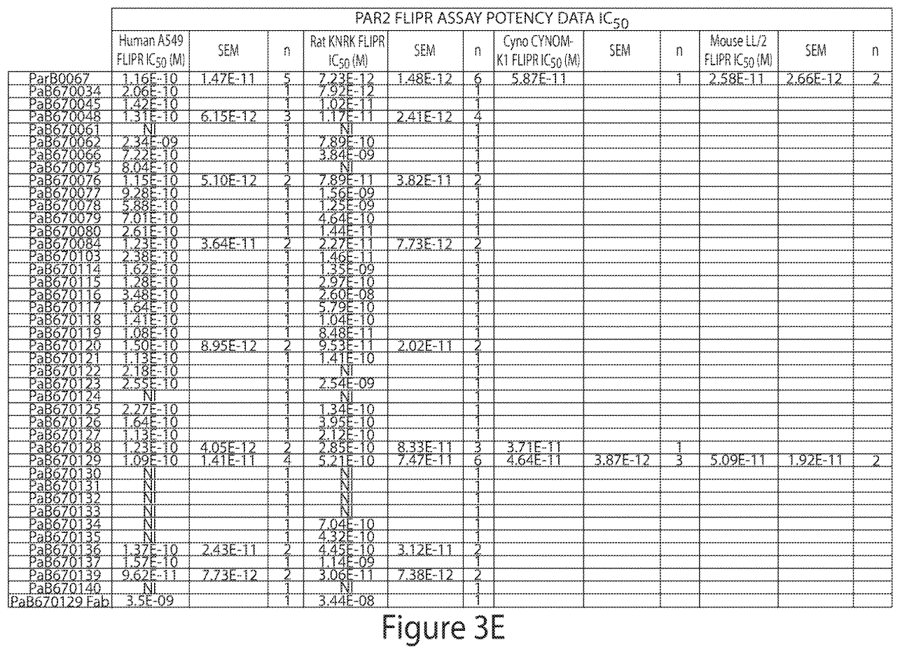

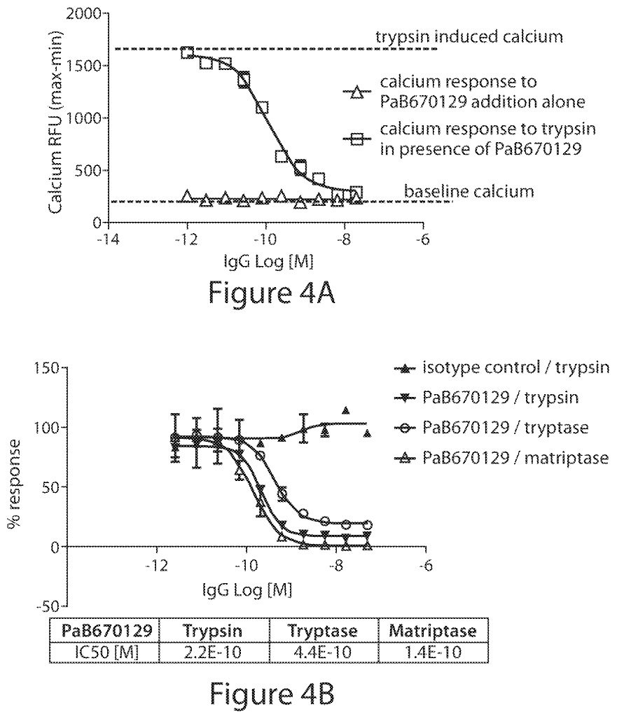

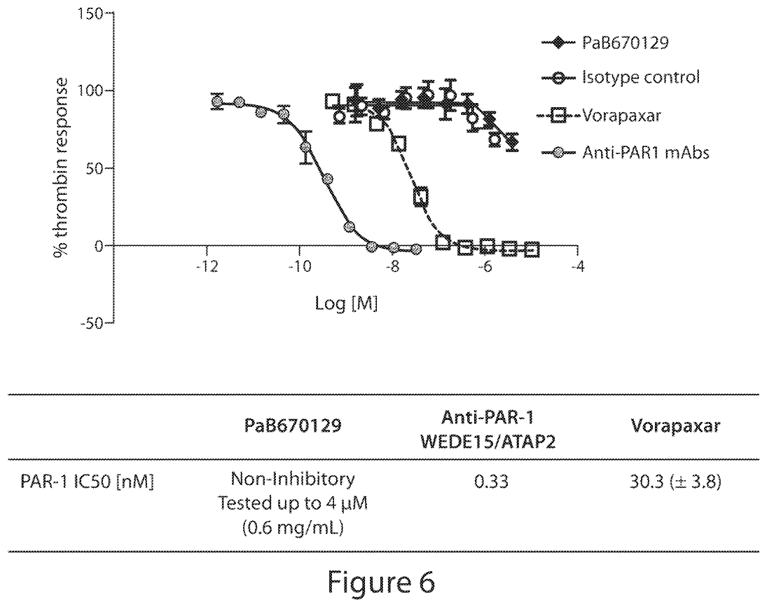

Results from human, cynomologus-monkey, rat, and mouse cells in these assays are provided in FIGS. 3A-3D, respectively, calculated PAR2 IC50s in FIG. 3E and FIG. 6 (PAR1 specificity data). IC50 calculations demonstrate that PaB670129 potently inhibits trypsin-induced PAR2 calcium responses in human, mouse, rat and cyno cells expressing endogenous PAR2 (FIG. 3E). Furthermore in human A549 cells, thrombin-induced PAR1 activation is not inhibited by PaB670129 but can be effectively blocked by the Vorapaxar and anti-PAR1 monoclonal antibodies (FIG. 6). These data demonstrate that PaB670129 is a potent and specific antagonist of PAR2. Application of PaB670129 alone to PAR2-expressing cells has no effect on basal cellular calcium levels, demonstrating that PaB670129 lacks any agonistic activity at PAR2 (FIG. 4A). Furthermore, PaB670129 potently antagonizes PAR2-evoked responses to diverse proteases including trypsin, tryptase and matriptase (FIG. 4B).

### Primary DRG Glial-neuronal PAR2 Calcium Assay

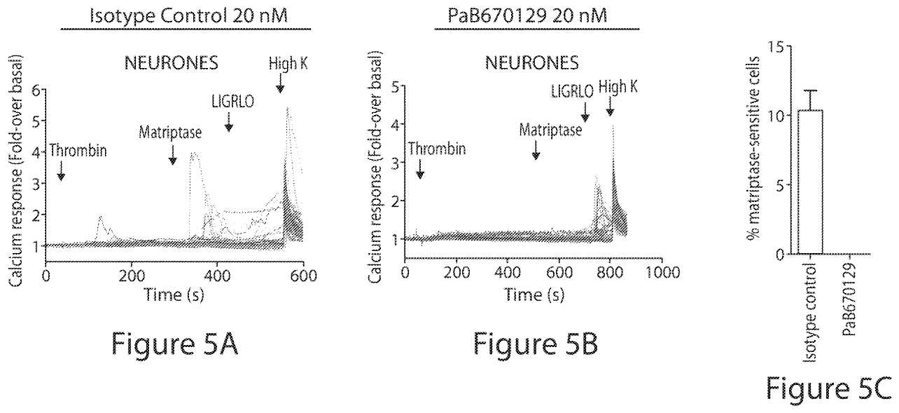

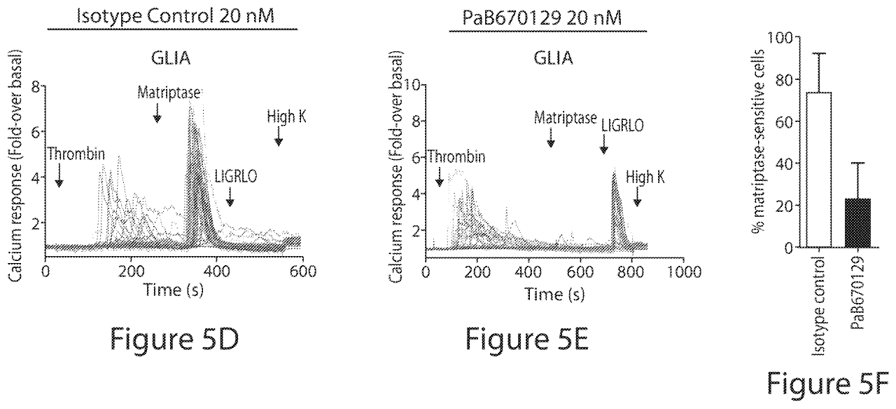

Dissociated cultures of Sprague dawley rat pup dorsal root ganglia (DRG) were prepared and grown on laminin and PDL-coated Tissue Culture Plates (Greiner Bio-One). Plates were incubated at 37 degrees for 24-72 hours before use in the assay. Cells were loaded with 2 μM Fura-2 calcium dye (Life Technologies). Cells were incubated in imaging buffer (HBSS, 20 mM HEPES, 0.1 mM Sulfinpyrazone, 10 μM PAR1 antagonist Vorapaxar) containing PAR2 antibodies at 20 nM. Intracellular calcium was then quantified by Fura-2 ratiometric imaging on an Olympus IX81 microscope equipped with a Xenon arc lamp exciting at 340 and 380 nm in response to application of the agonists thrombin (Sigma) and matriptase (R&D Systems) PAR2 activating peptide LIGRLO (SEQ ID NO: 832), (Peptides International) and high extracellular potassium (50 mM). Number of neurones versus glia was calculated per field of view (neurones defined as showing response to high potassium). Total matriptase-sensitive neurones and glia were then calculated per field of view. Results from the calcium assay are provided in FIGS. 5A-F and illustrate that the PaB670129 antibody effectively reduced sensitivity to matriptase in DRG neuronal (FIG. 5A-C) and non-neuronal cells (FIG. 5D-F).

### Example 3: Effects of an Anti-PAR2 Antibody in a Rat Model of Inflammatory Joint Pain

Intra-articular administration of Monosodium Iodoacetate (MIA) in the ipsilateral knee of Sprague Dawley rats leads to development of a robust and long-lasting hyperalgesia and allodynia associated initially with an inflammatory response. The development of these signs in this animal model are believed to be clinically relevant; reflecting the symptoms displayed by patients presenting with chronic inflammatory pain associated with underlying conditions such as osteoarthritis (OA) or rheumatoid arthritis (Bove et al., 2003; Fernihough et al., 2004; Kalbhen 1987). It has previously been demonstrated that (using weight-bearing as an end-point) the time course of MIA induced hyperalgesia follows a bi-phasic pattern with an early, predominantly inflammatory, component which is Cox-2 sensitive; and markedly reduced by the gold standard Celecoxib. This early inflammatory phase gives way to a more chronic pain phenotype which is Pregabalin (PGB) sensitive and Celecoxib insensitive suggesting an underlying more neuropathic-type component.

Weight bearing: Naive rats distribute their body weight equally between the two hind paws. However, when the injected (left) hind knee is inflamed and/or painful, the weight is re-distributed so that less weight is put on the affected limb (decrease in weight bearing on injured limb). Weight bearing through each hind limb is measured using a rat incapacitance tester (Linton Instruments, UK).

Sprague Dawley rats were placed in the incapacitance tester with the hind paws on separate sensors and the average force exerted by both hind limbs was recorded over 4 seconds.

Procedure: After delivery, rats underwent a minimum habituation period of 7 days prior to study commencement. Naïve rats were acclimatised to the procedure room in their home cages, with food and water available ad libitum. Habituations to the weight bearing chamber were performed over several days. Base line weight bearing readings were taken on the final day.

On Day 0, following the final baseline reading, animals were anaesthetised using isoflurane and oxygen mixed 3:1 in sterile conditions. The left knee area was shaved and cleaned with a dilute hibiscrub solution.

Osteoarthritis (OA) was induced via injection of MIA (Sigma, 12512) solution, 25 μl of 80 mg/ml, (2 mg) into the knee joint of the left hind leg. Sham animals were injected with saline. Animals were allowed to recover in a warmed environment, before being returned to their home cage.

Animals develop an inflammatory response post MIA and may guard and lick the affected area. Rats were therefore carefully monitored for unexpected signs of distress or severe pain, so that any animal displaying such signs could be culled immediately.

Animals were weighed daily for the first week and then every few days after. Weight bearing was assessed on Days 3, 7, 10 & 14, following injection of MIA, for development of chronic pain. On day 18, weight bearing measurements were taken and animals were ranked and randomised to treatment groups according to their MIA window in a Latin square design.

Antibody dosing regimen: Animals were treated with Par0067 (PAR2+ve) 10 mg/kg or isotype control (PAR2-ve) 10 mg/kg i.v. on day 18 and further weight bearing measurements were taken 4 hours and 1, 2, 6, 8, 10 & 14 days post antibody dosing.

Pregabalin & Celecoxib dosing regimen: Animals were dosed with PGB (30 mg/kg p.o; 2 ml/kg) or Celecoxib (50 mg/kg p.o; 2 ml/kg) daily on days 24, 25, 26, 27 and 28 post MIA injection. Weight bearing assessments were taken 1 hr post dosing on days 24, 26 and 28 and a further reading was taken post cessation of drug treatment on day 32.

On day 1, 2, 6, 10 and 14 post dosing, after weight bearing assessment, 400 μl of blood was taken via the tail vein for pk analysis from Antibody and Isotype control treated groups (n=5/group).

Evaluation of Study: Weight bearing (g) readings were taken for both right and left hind paws and the difference calculated. Data are expressed as % ratio ipsilateral/contralateral ((WB left/WB right)*100) (mean±s.c.m.).

Calculation: Ipsilateral reading/contralateral reading×100. Naïve WB difference-pre dose WB difference was defined as the MIA window.

Statistical analysis: Repeated measures ANOVA followed by Planned comparison test using In VivoStat (invivostat.co.uk), (p<0.05 considered significant). Data were analysed by comparing treatment groups to vehicle control group at each time point.

Injection of 2 mg MIA into the knee joint caused a marked inflammation and hypersensitivity response apparent from day 3 as detected by a shift in weight bearing between injured and non-injured hind paws. This MIA-induced hyperactivity response was still evident in all groups up until day 18 (and beyond for vehicle-treated control animals) at which point the first test agents were administered. Injection of saline had no effect on weight bearing.

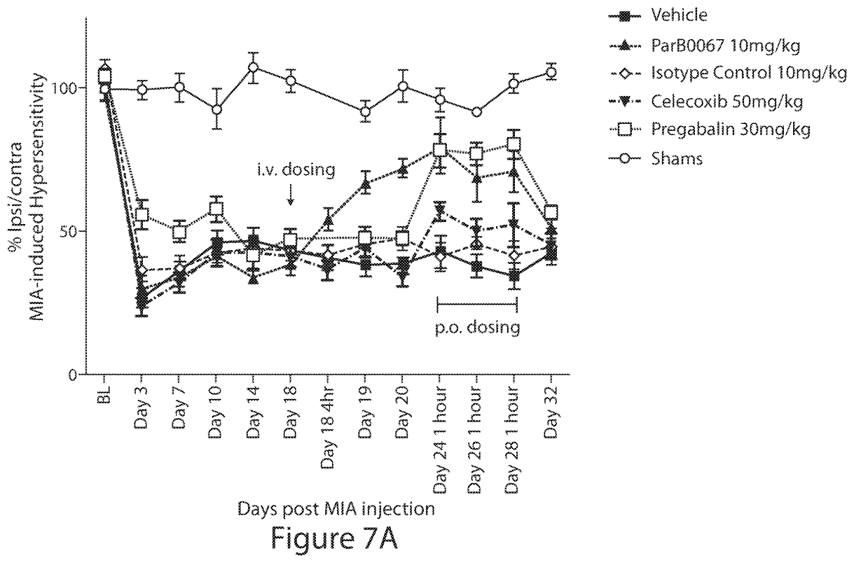

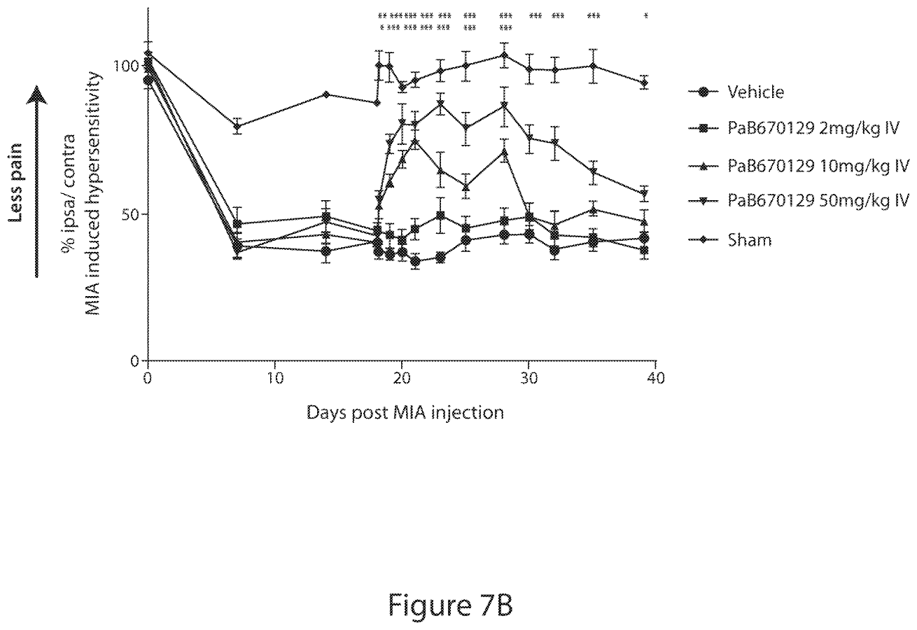

As demonstrated in FIG. 7A, a significant and marked reversal of hypersensitivity was seen after daily administration of Pregabalin (30 mg/kg) from day 24-28 with a weak residual effect still apparent after prior cessation of treatment on day 32. In contrast, daily administration of Celecoxib (50 mg/kg) from day 24-28 showed only a weak reversal of hypersensitivity at best. This pharmacological profile suggests that the hyperactivity observed during this phase of the MIA response is predominantly neuropathic, rather than inflammatory, in nature.

As also demonstrated in FIG. 7A, a significant reversal of hypersensitivity was seen with Par0067 from 4 hours post dose, through until day 28 (10 days post dose). No effect was seen with Isotype Control during the same time period. FIG. 7B illustrates the effect of treatment with different concentrations of Par0067.

### Example 4: The Effect of the PAR2 Antibody, PaB670129 on Reversal of Partial Nerve Ligation-Induced Mechanical Hyperalgesia in Female C57BL/6 Mice

### INTRODUCTION

Partial ligation of the sciatic nerve (PNL) as described by Seltzer (1990) is one of a number of nerve ligation models which are reported to serve as pre-clinical models of neuropathic pain. It produces a profound mechanical hyperalgesia which can be measured using an analgysemeter as described by Randall and Selitto (1957). This example describes the effects of the administration of the anti PAR2 antibody PaB670129, on hyperalgesia in this nerve injury/neuropathic pain model.

### Procedure

Sixty female C57BL/6 mice underwent insertion of transponders for identification purposes at least 5 days before the start of the study. Mechanical hyperalgesia was determined using an analgysemeter (Randall & Selitto 1957) (Ugo Basile). An increasing force was applied to the dorsal surface of each hind paw in turn until a withdrawal response was observed. The application of force was halted at this point and the weight in grams recorded. Data was expressed as withdrawal threshold in grams for ipsilateral and contralateral paws. Following the establishment of baseline readings, mice were divided into 2 groups with approximately equal ipsilateral/contralateral ratios which underwent surgery to partially ligate the sciatic nerve or served as sham operated controls. Operated mice were anaesthetised with isoflurane. Following this, approximately 1 cm of the left sciatic nerve was exposed by blunt dissection through an incision at the level of the mid thigh. A suture (8/0 Virgin Silk: Ethicon) was then passed through the dorsal third of the nerve and tied tightly. The incision was then closed using glue and the mice were allowed to recover for at least six days prior to commencement of testing. Sham operated mice underwent the same protocol, but following exposure of the nerve, the mice were sutured and allowed to recover.

Mice were tested for onset of hyperalgesia on days 7 and 10 post surgery. Any mice showing an ipsilateral/contralateral ratio of greater than 80% were classed as non-responders and removed from the study. Following testing on day 10, mice were further sub-divided into groups giving the final treatment groups;
A. Group 1: Sham operated+Isotype control 10 mg/kgs.c (N=10)
B. Group 2: Nerve Ligated+Isotype control 10 mg/kgs.c (N=9)
C. Group 3: Nerve ligated+Etanercept 0.3 mg/kg s.c. (N=9)
D. Group 4: Nerve ligated+PaB670129 3 mg/kg s.c. (N=9)
E. Group 5: Nerve ligated+PaB670129 10 mg/kg s.c. (N=9)
F. Group 6: Nerve ligated+PaB670129 50 mg/kg s.c. (N=9)

Mice were administered control or test molecules diluted in Phosphate Buffered Saline, (PBS) on day 13 and were re-tested for changes in mechanical hyperalgesia at 4 hours post dose and also on 1, 2, 4 and 7 days post dose.

### Data Analysis

Ipsilateral and contralateral readings were taken for each animal at each test time. Weight bearing through ipsilateral and contralateral hind limbs was expressed as a ratio and the group data were analysed (PRISM) using 2-way ANOVA and pairwise comparisons where appropriate were made using Tukey's test.

### Results

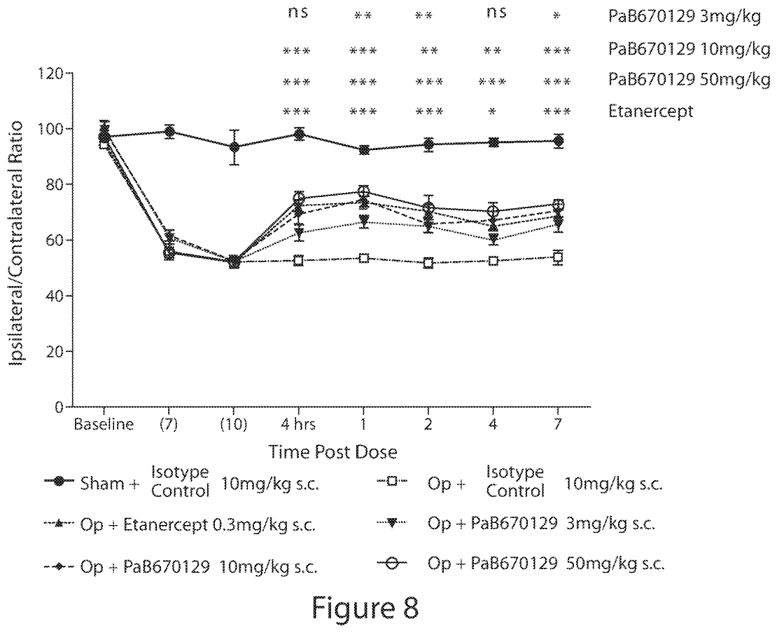

Partial ligation of the sciatic nerve caused a mechanical hyperalgesia which manifested as a significant reduction in the ipsilateral/contralateral ratio on day 7 and 10 when compared to sham operated controls. Following treatment with isotype control, operated mice did not show any change in the level of mechanical hyperalgesia from pre-dose levels indicating a lack of effect. The administration of the internal gold standard, etanercept (0.3 mg/kg s.c.) caused a significant reversal of the hyperalgesia from 4 hours through to 7 days post dose in agreement with the results seen in previous studies. PaB670129, caused a reversal of the ipsilateral/contralateral ratio in a dose related fashion with peak effects being seen at both 10 mg/kg and 50 mg/kg. The lowest dose of 3 mg/kg whilst significant at 1, 2 and 7 days post dose showed a smaller effect (see FIG. 8).

Partial ligation of the sciatic nerve induced a long lasting mechanical hyperalgesia consistent with previously reported results. Without wishing to be bound by theory, this is believed to serve as a pre-clinical correlate of the pain observed in neuropathic pain. The administration of PaB670129, showed a significant and dose related reversal of this hyperalgesia indicating a potential use of PAR2 antibodies in the treatment of neuropathic pain.

### Bibliography

Persic, L. et al. An integrated vector system for the eukaryotic expression of antibodies or their fragments after selection from phage display libraries. Gene 187, 9-18 (1997).
Reikofski J and Tao B Y (1992) Polymerase chain reaction (PCR) techniques for site-directed mutagenesis. Biotechnol Adv, 10 (4): 535-547.
Bove S E, Calcaterra S L, Brooker R M, Huber C M, Guzman R E, Juneau P L, et al. Weight bearing as a measure of disease progression and efficacy of anti-inflammatory compounds in a model of monosodium iodoacetate-induced osteoarthritis. Osteoarthritis Cartilage 2003; 11 (11): 821-30. eng.
Fernihough J, Gentry C, Malcangio M, Fox A, Rediske J, Pellas T, et al. Pain related behaviour in two models of osteoarthritis in the rat knee. Pain 2004; 112 (1-2): 83-93. eng.
Kalbhen D A. Chemical model of osteoarthritis—a pharmacological evaluation. J Rheumatol 1987; 14 Spec No: 130-1. eng.
Clark R A, Shoaib M, Hewitt K N, Stanford S C, Bate S T (2012)., A comparison of In VivoStat with other statistical software packages for analysis of data generated from animal experiments, J Psychopharmacology, 26 (8), 1136-1142.
Myska Improving Biosensor Analysis. Journal of Molecular Recognition. 1999.
D.G. Myska Improving Biosensor Analysis. Journal of Molecular Recognition. 1999; 12:279-284.
Myska D G, Improving Biosensor Analysis. Journal of Molecular Recognition. 1999; 12:279-284.
A. W. Drake, M. L. Tang, G. A. Papalia, G. Landes, M. Haak-Frendscho, S. L. Klakamp, Biacore surface matrix effects on the binding kinetics and affinity of an antigen/antibody complex, Anal. Biochem. 429 (2012) 58-69
Pace C N, Vajdos F, Fee L, Grisley G and Grey T, How to measure and predict the molar absorption coefficient of a protein, Protein Sci. 1995; 4:2411-2423.
Spooner J, Keen J, Nayyar K, Birkett N, Bond N, Bannister D, Tigue N, Higazi D, Kemp B, Vaughan T, Kippen A, Buchanan A. (2015) Evaluation of strategies to control Fab light chain dimer during mammalian expression and purification: A universal one-step process for purification of correctly assembled Fab. Biotechnol Bioeng. 112:1472-7.
Daramola O, Stevenson J, Dean G, Hatton D, Pettman G, Holmes W, Field R (2014) A high yielding CHO transient system: co-expression of genes encoding EBNA-1 and GS enhances transient protein expression. Biotechnol Prog. 30 (1): 132-41.
Mach H, Middaugh C R, Lewis R V (1992) Statistical determination of the average values of the extinction coefficients of tryptophan and tyrosine in native proteins. Anal. Biochem. 200 (1): 74-80.
Seltzer Z, Dubner R, Shir Y (1990). A novel behavioural model of neuropathic pain disorders produced in rats by partial sciatic nerve injury. Pain 43:205-218
Randall L O, Selitto J J (1957). A method for measurement of analgesic activity on inflamed tissue. Arch Int Pharmacodyn Ther. 111 (4): 409-419

### SEQUENCE LISTING

| SEQ | | |
|---|---|---|
| ID | | |
| NO: | clone name | Type |
| 1 | ZZ15D2-D02 (Par0067) | VH DNA |
| 2 | ZZ15D2-D02 (Par0067) | VH PRT |
| 3 | ZZ15D2-D02 (Par0067) | CDR1 PRT |
| 4 | ZZ15D2-D02 (Par0067) | CDR2 PRT |
| 5 | ZZ15D2-D02 (Par0067) | CDR3 PRT |
| 6 | ZZ15D2-D02 (Par0067) | VL DNA |
| 7 | ZZ15D2-D02 (Par0067) | VL PRT |
| 8 | ZZ15D2-D02 (Par0067) | CDR1 PRT |
| 9 | ZZ15D2-D02 (Par0067) | CDR2 PRT |
| 10 | ZZ15D2-D02 (Par0067) | CDR3 PRT |
| 11 | ZZ1RUE−F02 (PaB670129) | VH DNA |
| 12 | ZZ1RUE−F02 (PaB670129) | VH PRT |
| 13 | ZZ1RUE−F02 (PaB670129) | CDR1 PRT |
| 14 | ZZ1RUE−F02 (PaB670129) | CDR2 PRT |
| 15 | ZZ1RUE−F02 (PaB670129) | CDR3 PRT |
| 16 | ZZ1RUE−F02 (PaB670129) | VL DNA |
| 17 | ZZ1RUE−F02 (PaB670129) | VL PRT |
| 18 | ZZ1RUE−F02 (PaB670129) | CDR1 PRT |
| 19 | ZZ1RUE−F02 (PaB670129) | CDR2 PRT |
| 20 | ZZ1RUE−F02 (PaB670129) | CDR3 PRT |
| 21 | ZZ1DRB-B08 (PaB670010) | VH DNA |
| 22 | ZZ1DRB-B08 (PaB670010) | VH PRT |
| 23 | ZZ1DRB-B08 (PaB670010) | CDR1 PRT |
| 24 | ZZ1DRB-B08 (PaB670010) | CDR2 PRT |
| 25 | ZZ1DRB-B08 (PaB670010) | CDR3 PRT |
| 26 | ZZ1DRB-B08 (PaB670010) | VL DNA |
| 27 | ZZ1DRB-B08 (PaB670010) | VL PRT |
| 28 | ZZ1DRB-B08 (PaB670010) | CDR1 PRT |
| 29 | ZZ1DRB-B08 (PaB670010) | CDR2 PRT |
| 30 | ZZ1DRB-B08 (PaB670010) | CDR3 PRT |
| 31 | ZZ1IGD-D05 (PaB670020) | VH DNA |
| 32 | ZZ1IGD-D05 (PaB670020) | VH PRT |
| 33 | ZZ1IGD-D05 (PaB670020) | CDR1 PRT |
| 34 | ZZ1IGD-D05 (PaB670020) | CDR2 PRT |
| 35 | ZZ1IGD-D05 (PaB670020) | CDR3 PRT |
| 36 | ZZ1IGD-D05 (PaB670020) | VL DNA |
| 37 | ZZ1IGD-D05 (PaB670020) | VL PRT |
| 38 | ZZ1IGD-D05 (PaB670020) | CDR1 PRT |
| 39 | ZZ1IGD-D05 (PaB670020) | CDR2 PRT |
| 40 | ZZ1IGD-D05 (PaB670020) | CDR3 PRT |
| 41 | ZZ1IGF-B11 (PaB670034) | VH DNA |
| 42 | ZZ1IGF-B11 (PaB670034) | VH PRT |
| 43 | ZZ1IGF-B11 (PaB670034) | CDR1 PRT |
| 44 | ZZ1IGF-B11 (PaB670034) | CDR2 PRT |
| 45 | ZZ1IGF-B11 (PaB670034) | CDR3 PRT |
| 46 | ZZ1IGF-B11 (PaB670034) | VL DNA |
| 47 | ZZ1IGF-B11 (PaB670034) | VL PRT |
| 48 | ZZ1IGF-B11 (PaB670034) | CDR1 PRT |
| 49 | ZZ1IGF-B11 (PaB670034) | CDR2 PRT |
| 50 | ZZ1IGF-B11 (PaB670034) | CDR3 PRT |
| 51 | ZZ1KX3-F01 (PaB670045) | VH DNA |
| 52 | ZZ1KX3-F01 (PaB670045) | VH PRT |
| 53 | ZZ1KX3-F01 (PaB670045) | CDR1 PRT |
| 54 | ZZ1KX3-F01 (PaB670045) | CDR2 PRT |
| 55 | ZZ1KX3-F01 (PaB670045) | CDR3 PRT |
| 56 | ZZ1KX3-F01 (PaB670045) | VL DNA |
| 57 | ZZ1KX3-F01 (PaB670045) | VL PRT |
| 58 | ZZ1KX3-F01 (PaB670045) | CDR1 PRT |
| 59 | ZZ1KX3-F01 (PaB670045) | CDR2 PRT |
| 60 | ZZ1KX3-F01 (PaB670045) | CDR3 PRT |
| 61 | ZZ1KX4-E11 (PaB670048) | VH DNA |
| 62 | ZZ1KX4-E11 (PaB670048) | VH PRT |
| 63 | ZZ1KX4-E11 (PaB670048) | CDR1 PRT |
| 64 | ZZ1KX4-E11 (PaB670048) | CDR2 PRT |
| 65 | ZZ1KX4-E11 (PaB670048) | CDR3 PRT |
| 66 | ZZ1KX4-E11 (PaB670048) | VL DNA |
| 67 | ZZ1KX4-E11 (PaB670048) | VL PRT |
| 68 | ZZ1KX4-E11 (PaB670048) | CDR1 PRT |
| 69 | ZZ1KX4-E11 (PaB670048) | CDR2 PRT |
| 70 | ZZ1KX4-E11 (PaB670048) | CDR3 PRT |
| 71 | ZZ1KX6-B09 (PaB670064) | VH DNA |
| 72 | ZZ1KX6-B09 (PaB670064) | VH PRT |
| 73 | ZZ1KX6-B09 (PaB670064) | CDR1 PRT |
| 74 | ZZ1KX6-B09 (PaB670064) | CDR2 PRT |
| 75 | ZZ1KX6-B09 (PaB670064) | CDR3 PRT |
| 76 | ZZ1KX6-B09 (PaB670064) | VL DNA |
| 77 | ZZ1KX6-B09 (PaB670064) | VL PRT |
| 78 | ZZ1KX6-B09 (PaB670064) | CDR1 PRT |
| 79 | ZZ1KX6-B09 (PaB670064) | CDR2 PRT |
| 80 | ZZ1KX6-B09 (PaB670064) | CDR3 PRT |
| 81 | ZZ1KX6-D05 (PaB670066) | VH DNA |
| 82 | ZZ1KX6-D05 (PaB670066) | VH PRT |
| 83 | ZZ1KX6-D05 (PaB670066) | CDR1 PRT |
| 84 | ZZ1KX6-D05 (PaB670066) | CDR2 PRT |
| 85 | ZZ1KX6-D05 (PaB670066) | CDR3 PRT |
| 86 | ZZ1KX6-D05 (PaB670066) | VL DNA |
| 87 | ZZ1KX6-D05 (PaB670066) | VL PRT |
| 88 | ZZ1KX6-D05 (PaB670066) | CDR1 PRT |
| 89 | ZZ1KX6-D05 (PaB670066) | CDR2 PRT |
| 90 | ZZ1KX6-D05 (PaB670066) | CDR3 PRT |
| 91 | ZZ1KXE−A05 (PaB670067) | VH DNA |
| 92 | ZZ1KXE−A05 (PaB670067) | VH PRT |
| 93 | ZZ1KXE−A05 (PaB670067) | CDR1 PRT |
| 94 | ZZ1KXE−A05 (PaB670067) | CDR2 PRT |
| 95 | ZZ1KXE−A05 (PaB670067) | CDR3 PRT |
| 96 | ZZ1KXE−A05 (PaB670067) | VL DNA |
| 97 | ZZ1KXE−A05 (PaB670067) | VL PRT |
| 98 | ZZ1KXE−A05 (PaB670067) | CDR1 PRT |
| 99 | ZZ1KXE−A05 (PaB670067) | CDR2 PRT |
| 100 | ZZ1KXE−A05 (PaB670067) | CDR3 PRT |
| 101 | ZZ1KXE−B01 (PaB670068) | VH DNA |
| 102 | ZZ1KXE−B01 (PaB670068) | VH PRT |
| 103 | ZZ1KXE−B01 (PaB670068) | CDR1 PRT |
| 104 | ZZ1KXE−B01 (PaB670068) | CDR2 PRT |
| 105 | ZZ1KXE−B01 (PaB670068) | CDR3 PRT |
| 106 | ZZ1KXE−B01 (PaB670068) | VL DNA |
| 107 | ZZ1KXE−B01 (PaB670068) | VL PRT |
| 108 | ZZ1KXE−B01 (PaB670068) | CDR1 PRT |
| 109 | ZZ1KXE−B01 (PaB670068) | CDR2 PRT |
| 110 | ZZ1KXE−B01 (PaB670068) | CDR3 PRT |
| 111 | ZZ1KXE−D06 (PaB670070) | VH DNA |
| 112 | ZZ1KXE−D06 (PaB670070) | VH PRT |
| 113 | ZZ1KXE−D06 (PaB670070) | CDR1 PRT |
| 114 | ZZ1KXE−D06 (PaB670070) | CDR2 PRT |
| 115 | ZZ1KXE−D06 (PaB670070) | CDR3 PRT |
| 116 | ZZ1KXE−D06 (PaB670070) | VL DNA |
| 117 | ZZ1KXE−D06 (PaB670070) | VL PRT |
| 118 | ZZ1KXE−D06 (PaB670070) | CDR1 PRT |
| 119 | ZZ1KXE−D06 (PaB670070) | CDR2 PRT |
| 120 | ZZ1KXE−D06 (PaB670070) | CDR3 PRT |
| 121 | ZZ1L3F-A02 (PaB670071) | VH DNA |
| 122 | ZZ1L3F-A02 (PaB670071) | VH PRT |
| 123 | ZZ1L3F-A02 (PaB670071) | CDR1 PRT |
| 124 | ZZ1L3F-A02 (PaB670071) | CDR2 PRT |
| 125 | ZZ1L3F-A02 (PaB670071) | CDR3 PRT |
| 126 | ZZ1L3F-A02 (PaB670071) | VL DNA |
| 127 | ZZ1L3F-A02 (PaB670071) | VL PRT |
| 128 | ZZ1L3F-A02 (PaB670071) | CDR1 PRT |
| 129 | ZZ1L3F-A02 (PaB670071) | CDR2 PRT |
| 130 | ZZ1L3F-A02 (PaB670071) | CDR3 PRT |
| 131 | ZZ1L3F-H03 (PaB670073) | VH DNA |
| 132 | ZZ1L3F-H03 (PaB670073) | VH PRT |
| 133 | ZZ1L3F-H03 (PaB670073) | CDR1 PRT |
| 134 | ZZ1L3F-H03 (PaB670073) | CDR2 PRT |
| 135 | ZZ1L3F-H03 (PaB670073) | CDR3 PRT |
| 136 | ZZ1L3F-H03 (PaB670073) | VL DNA |
| 137 | ZZ1L3F-H03 (PaB670073) | VL PRT |
| 138 | ZZ1L3F-H03 (PaB670073) | CDR1 PRT |
| 139 | ZZ1L3F-H03 (PaB670073) | CDR2 PRT |
| 140 | ZZ1L3F-H03 (PaB670073) | CDR3 PRT |
| 141 | ZZ1NHH-A05 (PaB670075) | VH DNA |
| 142 | ZZ1NHH-A05 (PaB670075) | VH PRT |
| 143 | ZZ1NHH-A05 (PaB670075) | CDR1 PRT |
| 144 | ZZ1NHH-A05 (PaB670075) | CDR2 PRT |
| 145 | ZZ1NHH-A05 (PaB670075) | CDR3 PRT |
| 146 | ZZ1NHH-A05 (PaB670075) | VL DNA |
| 147 | ZZ1NHH-A05 (PaB670075) | VL PRT |
| 148 | ZZ1NHH-A05 (PaB670075) | CDR1 PRT |
| 149 | ZZ1NHH-A05 (PaB670075) | CDR2 PRT |
| 150 | ZZ1NHH-A05 (PaB670075) | CDR3 PRT |
| 151 | ZZ1NHH-F09 (PaB670076) | VH DNA |
| 152 | ZZ1NHH-F09 (PaB670076) | VH PRT |
| 153 | ZZ1NHH-F09 (PaB670076) | CDR1 PRT |
| 154 | ZZ1NHH-F09 (PaB670076) | CDR2 PRT |
| 155 | ZZ1NHH-F09 (PaB670076) | CDR3 PRT |
| 156 | ZZ1NHH-F09 (PaB670076) | VL DNA |
| 157 | ZZ1NHH-F09 (PaB670076) | VL PRT |
| 158 | ZZ1NHH-F09 (PaB670076) | CDR1 PRT |
| 159 | ZZ1NHH-F09 (PaB670076) | CDR2 PRT |
| 160 | ZZ1NHH-F09 (PaB670076) | CDR3 PRT |
| 161 | ZZ1OZJ-C11 (PaB670077) | VH DNA |
| 162 | ZZ1OZJ-C11 (PaB670077) | VH PRT |
| 163 | ZZ1OZJ-C11 (PaB670077) | CDR1 PRT |
| 164 | ZZ1OZJ-C11 (PaB670077) | CDR2 PRT |
| 165 | ZZ1OZJ-C11 (PaB670077) | CDR3 PRT |
| 166 | ZZ1OZJ-C11 (PaB670077) | VL DNA |
| 167 | ZZ1OZJ-C11 (PaB670077) | VL PRT |
| 168 | ZZ1OZJ-C11 (PaB670077) | CDR1 PRT |
| 169 | ZZ1OZJ-C11 (PaB670077) | CDR2 PRT |
| 170 | ZZ1OZJ-C11 (PaB670077) | CDR3 PRT |
| 171 | ZZ1OZJ-G03 (PaB670078) | VH DNA |
| 172 | ZZ1OZJ-G03 (PaB670078) | VH PRT |
| 173 | ZZ1OZJ-G03 (PaB670078) | CDR1 PRT |
| 174 | ZZ1OZJ-G03 (PaB670078) | CDR2 PRT |
| 175 | ZZ1OZJ-G03 (PaB670078) | CDR3 PRT |
| 176 | ZZ1OZJ-G03 (PaB670078) | VL DNA |
| 177 | ZZ1OZJ-G03 (PaB670078) | VL PRT |
| 178 | ZZ1OZJ-G03 (PaB670078) | CDR1 PRT |
| 179 | ZZ1OZJ-G03 (PaB670078) | CDR2 PRT |
| 180 | ZZ1OZJ-G03 (PaB670078) | CDR3 PRT |
| 181 | ZZ1OZJ-G05 (PaB670079) | VH DNA |
| 182 | ZZ1OZJ-G05 (PaB670079) | VH PRT |
| 183 | ZZ1OZJ-G05 (PaB670079) | CDR1 PRT |
| 184 | ZZ1OZJ-G05 (PaB670079) | CDR2 PRT |
| 185 | ZZ1OZJ-G05 (PaB670079) | CDR3 PRT |
| 186 | ZZ1OZJ-G05 (PaB670079) | VL DNA |
| 187 | ZZ1OZJ-G05 (PaB670079) | VL PRT |
| 188 | ZZ1OZJ-G05 (PaB670079) | CDR1 PRT |
| 189 | ZZ1OZJ-G05 (PaB670079) | CDR2 PRT |
| 190 | ZZ1OZJ-G05 (PaB670079) | CDR3 PRT |
| 191 | PaB670080 | VH DNA |
| 192 | PaB670080 | VH PRT |
| 193 | PaB670080 | CDR1 PRT |
| 194 | PaB670080 | CDR2 PRT |
| 195 | PaB670080 | CDR3 PRT |
| 196 | PaB670080 | VL DNA |
| 197 | PaB670080 | VL PRT |
| 198 | PaB670080 | CDR1 PRT |
| 199 | PaB670080 | CDR2 PRT |
| 200 | PaB670080 | CDR3 PRT |
| 201 | ZZ1OZA-C01 (PaB670081) | VH DNA |
| 202 | ZZ1OZA-C01 (PaB670081) | VH PRT |
| 203 | ZZ1OZA-C01 (PaB670081) | CDR1 PRT |
| 204 | ZZ1OZA-C01 (PaB670081) | CDR2 PRT |
| 205 | ZZ1OZA-C01 (PaB670081) | CDR3 PRT |
| 206 | ZZ1OZA-C01 (PaB670081) | VL DNA |
| 207 | ZZ1OZA-C01 (PaB670081) | VL PRT |
| 208 | ZZ1OZA-C01 (PaB670081) | CDR1 PRT |
| 209 | ZZ1OZA-C01 (PaB670081) | CDR2 PRT |
| 210 | ZZ1OZA-C01 (PaB670081) | CDR3 PRT |
| 211 | ZZ1OZA-D02 (PaB670082) | VH DNA |
| 212 | ZZ1OZA-D02 (PaB670082) | VH PRT |
| 213 | ZZ1OZA-D02 (PaB670082) | CDR1 PRT |
| 214 | ZZ1OZA-D02 (PaB670082) | CDR2 PRT |
| 215 | ZZ1OZA-D02 (PaB670082) | CDR3 PRT |
| 216 | ZZ1OZA-D02 (PaB670082) | VL DNA |
| 217 | ZZ1OZA-D02 (PaB670082) | VL PRT |
| 218 | ZZ1OZA-D02 (PaB670082) | CDR1 PRT |
| 219 | ZZ1OZA-D02 (PaB670082) | CDR2 PRT |
| 220 | ZZ1OZA-D02 (PaB670082) | CDR3 PRT |
| 221 | ZZ1OZB-H05 (PaB670083) | VH DNA |
| 222 | ZZ1OZB-H05 (PaB670083) | VH PRT |
| 223 | ZZ1OZB-H05 (PaB670083) | CDR1 PRT |
| 224 | ZZ1OZB-H05 (PaB670083) | CDR2 PRT |
| 225 | ZZ1OZB-H05 (PaB670083) | CDR3 PRT |
| 226 | ZZ1OZB-H05 (PaB670083) | VL DNA |
| 227 | ZZ1OZB-H05 (PaB670083) | VL PRT |
| 228 | ZZ1OZB-H05 (PaB670083) | CDR1 PRT |
| 229 | ZZ1OZB-H05 (PaB670083) | CDR2 PRT |
| 230 | ZZ1OZB-H05 (PaB670083) | CDR3 PRT |
| 231 | ZZ1PXA-A05 (PaB670084) | VH DNA |
| 232 | ZZ1PXA-A05 (PaB670084) | VH PRT |
| 233 | ZZ1PXA-A05 (PaB670084) | CDR1 PRT |
| 234 | ZZ1PXA-A05 (PaB670084) | CDR2 PRT |
| 235 | ZZ1PXA-A05 (PaB670084) | CDR3 PRT |
| 236 | ZZ1PXA-A05 (PaB670084) | VL DNA |
| 237 | ZZ1PXA-A05 (PaB670084) | VL PRT |
| 238 | ZZ1PXA-A05 (PaB670084) | CDR1 PRT |
| 239 | ZZ1PXA-A05 (PaB670084) | CDR2 PRT |
| 240 | ZZ1PXA-A05 (PaB670084) | CDR3 PRT |
| 241 | ZZ1ODR-A02 (PaB670085) | VH DNA |
| 242 | ZZ1ODR-A02 (PaB670085) | VH PRT |
| 243 | ZZ1ODR-A02 (PaB670085) | CDR1 PRT |
| 244 | ZZ1ODR-A02 (PaB670085) | CDR2 PRT |
| 245 | ZZ1ODR-A02 (PaB670085) | CDR3 PRT |
| 246 | ZZ1ODR-A02 (PaB670085) | VL DNA |
| 247 | ZZ1ODR-A02 (PaB670085) | VL PRT |
| 248 | ZZ1ODR-A02 (PaB670085) | CDR1 PRT |
| 249 | ZZ1ODR-A02 (PaB670085) | CDR2 PRT |
| 250 | ZZ1ODR-A02 (PaB670085) | CDR3 PRT |
| 251 | ZZ1ODR-B05 (PaB670087) | VH DNA |
| 252 | ZZ1ODR-B05 (PaB670087) | VH PRT |
| 253 | ZZ1ODR-B05 (PaB670087) | CDR1 PRT |
| 254 | ZZ1ODR-B05 (PaB670087) | CDR2 PRT |
| 255 | ZZ1ODR-B05 (PaB670087) | CDR3 PRT |
| 256 | ZZ1ODR-B05 (PaB670087) | VL DNA |
| 257 | ZZ1ODR-B05 (PaB670087) | VL PRT |
| 258 | ZZ1ODR-B05 (PaB670087) | CDR1 PRT |
| 259 | ZZ1ODR-B05 (PaB670087) | CDR2 PRT |
| 260 | ZZ1ODR-B05 (PaB670087) | CDR3 PRT |
| 261 | ZZ1ODR-B11 (PaB670088) | VH DNA |
| 262 | ZZ1ODR-B11 (PaB670088) | VH PRT |
| 263 | ZZ1ODR-B11 (PaB670088) | CDR1 PRT |
| 264 | ZZ1ODR-B11 (PaB670088) | CDR2 PRT |
| 265 | ZZ1ODR-B11 (PaB670088) | CDR3 PRT |
| 266 | ZZ1ODR-B11 (PaB670088) | VL DNA |
| 267 | ZZ1ODR-B11 (PaB670088) | VL PRT |
| 268 | ZZ1ODR-B11 (PaB670088) | CDR1 PRT |
| 269 | ZZ1ODR-B11 (PaB670088) | CDR2 PRT |
| 270 | ZZ1ODR-B11 (PaB670088) | CDR3 PRT |
| 271 | ZZ1ODR-C05 (PaB670089) | VH DNA |
| 272 | ZZ1ODR-C05 (PaB670089) | VH PRT |
| 273 | ZZ1ODR-C05 (PaB670089) | CDR1 PRT |
| 274 | ZZ1ODR-C05 (PaB670089) | CDR2 PRT |
| 275 | ZZ1ODR-C05 (PaB670089) | CDR3 PRT |
| 276 | ZZ1ODR-C05 (PaB670089) | VL DNA |
| 277 | ZZ1ODR-C05 (PaB670089) | VL PRT |
| 278 | ZZ1ODR-C05 (PaB670089) | CDR1 PRT |
| 279 | ZZ1ODR-C05 (PaB670089) | CDR2 PRT |
| 280 | ZZ1ODR-C05 (PaB670089) | CDR3 PRT |
| 281 | ZZ1ODR-F02 (PaB670090) | VH DNA |
| 282 | ZZ1ODR-F02 (PaB670090) | VH PRT |
| 283 | ZZ1ODR-F02 (PaB670090) | CDR1 PRT |
| 284 | ZZ1ODR-F02 (PaB670090) | CDR2 PRT |
| 285 | ZZ1ODR-F02 (PaB670090) | CDR3 PRT |
| 286 | ZZ1ODR-F02 (PaB670090) | VL DNA |
| 287 | ZZ1ODR-F02 (PaB670090) | VL PRT |
| 288 | ZZ1ODR-F02 (PaB670090) | CDR1 PRT |
| 289 | ZZ1ODR-F02 (PaB670090) | CDR2 PRT |
| 290 | ZZ1ODR-F02 (PaB670090) | CDR3 PRT |
| 291 | ZZ1ODR-G02 (PaB670091) | VH DNA |
| 292 | ZZ1ODR-G02 (PaB670091) | VH PRT |
| 293 | ZZ1ODR-G02 (PaB670091) | CDR1 PRT |
| 294 | ZZ1ODR-G02 (PaB670091) | CDR2 PRT |
| 295 | ZZ1ODR-G02 (PaB670091) | CDR3 PRT |
| 296 | ZZ1ODR-G02 (PaB670091) | VL DNA |
| 297 | ZZ1ODR-G02 (PaB670091) | VL PRT |
| 298 | ZZ1ODR-G02 (PaB670091) | CDR1 PRT |
| 299 | ZZ1ODR-G02 (PaB670091) | CDR2 PRT |
| 300 | ZZ1ODR-G02 (PaB670091) | CDR3 PRT |
| 301 | ZZ1ODR-G11 (PaB670092) | VH DNA |
| 302 | ZZ1ODR-G11 (PaB670092) | VH PRT |
| 303 | ZZ1ODR-G11 (PaB670092) | CDR1 PRT |
| 304 | ZZ1ODR-G11 (PaB670092) | CDR2 PRT |
| 305 | ZZ1ODR-G11 (PaB670092) | CDR3 PRT |
| 306 | ZZ1ODR-G11 (PaB670092) | VL DNA |
| 307 | ZZ1ODR-G11 (PaB670092) | VL PRT |
| 308 | ZZ1ODR-G11 (PaB670092) | CDR1 PRT |
| 309 | ZZ1ODR-G11 (PaB670092) | CDR2 PRT |
| 310 | ZZ1ODR-G11 (PaB670092) | CDR3 PRT |
| 311 | ZZ1ODR-H04 (PaB670093) | VH DNA |
| 312 | ZZ1ODR-H04 (PaB670093) | VH PRT |
| 313 | ZZ1ODR-H04 (PaB670093) | CDR1 PRT |
| 314 | ZZ1ODR-H04 (PaB670093) | CDR2 PRT |
| 315 | ZZ1ODR-H04 (PaB670093) | CDR3 PRT |
| 316 | ZZ1ODR-H04 (PaB670093) | VL DNA |
| 317 | ZZ1ODR-H04 (PaB670093) | VL PRT |
| 318 | ZZ1ODR-H04 (PaB670093) | CDR1 PRT |
| 319 | ZZ1ODR-H04 (PaB670093) | CDR2 PRT |
| 320 | ZZ1ODR-H04 (PaB670093) | CDR3 PRT |
| 321 | ZZ1ODS-B08 (PaB670094) | VH DNA |
| 322 | ZZ1ODS-B08 (PaB670094) | VH PRT |
| 323 | ZZ1ODS-B08 (PaB670094) | CDR1 PRT |
| 324 | ZZ1ODS-B08 (PaB670094) | CDR2 PRT |
| 325 | ZZ1ODS-B08 (PaB670094) | CDR3 PRT |
| 326 | ZZ1ODS-B08 (PaB670094) | VL DNA |
| 327 | ZZ1ODS-B08 (PaB670094) | VL PRT |
| 328 | ZZ1ODS-B08 (PaB670094) | CDR1 PRT |
| 329 | ZZ1ODS-B08 (PaB670094) | CDR2 PRT |
| 330 | ZZ1ODS-B08 (PaB670094) | CDR3 PRT |
| 331 | ZZ1ODS-H05 (PaB670095) | VH DNA |
| 332 | ZZ1ODS-H05 (PaB670095) | VH PRT |
| 333 | ZZ1ODS-H05 (PaB670095) | CDR1 PRT |
| 334 | ZZ1ODS-H05 (PaB670095) | CDR2 PRT |
| 335 | ZZ1ODS-H05 (PaB670095) | CDR3 PRT |
| 336 | ZZ1ODS-H05 (PaB670095) | VL DNA |
| 337 | ZZ1ODS-H05 (PaB670095) | VL PRT |
| 338 | ZZ1ODS-H05 (PaB670095) | CDR1 PRT |
| 339 | ZZ1ODS-H05 (PaB670095) | CDR2 PRT |
| 340 | ZZ1ODS-H05 (PaB670095) | CDR3 PRT |
| 341 | ZZ1ODT-E11 (PaB670097) | VH DNA |
| 342 | ZZ1ODT-E11 (PaB670097) | VH PRT |
| 343 | ZZ1ODT-E11 (PaB670097) | CDR1 PRT |
| 344 | ZZ1ODT-E11 (PaB670097) | CDR2 PRT |
| 345 | ZZ1ODT-E11 (PaB670097) | CDR3 PRT |
| 346 | ZZ1ODT-E11 (PaB670097) | VL DNA |
| 347 | ZZ1ODT-E11 (PaB670097) | VL PRT |
| 348 | ZZ1ODT-E11 (PaB670097) | CDR1 PRT |
| 349 | ZZ1ODT-E11 (PaB670097) | CDR2 PRT |
| 350 | ZZ1ODT-E11 (PaB670097) | CDR3 PRT |
| 351 | ZZ1ODT-G01 (PaB670098) | VH DNA |
| 352 | ZZ1ODT-G01 (PaB670098) | VH PRT |
| 353 | ZZ1ODT-G01 (PaB670098) | CDR1 PRT |
| 354 | ZZ1ODT-G01 (PaB670098) | CDR2 PRT |
| 355 | ZZ1ODT-G01 (PaB670098) | CDR3 PRT |
| 356 | ZZ1ODT-G01 (PaB670098) | VL DNA |
| 357 | ZZ1ODT-G01 (PaB670098) | VL PRT |
| 358 | ZZ1ODT-G01 (PaB670098) | CDR1 PRT |
| 359 | ZZ1ODT-G01 (PaB670098) | CDR2 PRT |
| 360 | ZZ1ODT-G01 (PaB670098) | CDR3 PRT |
| 361 | PaB670099 | VH DNA |
| 362 | PaB670099 | VH PRT |
| 363 | PaB670099 | CDR1 PRT |
| 364 | PaB670099 | CDR2 PRT |
| 365 | PaB670099 | CDR3 PRT |
| 366 | PaB670099 | VL DNA |
| 367 | PaB670099 | VL PRT |
| 368 | PaB670099 | CDR1 PRT |
| 369 | PaB670099 | CDR2 PRT |
| 370 | PaB670099 | CDR3 PRT |
| 371 | PaB670100 | VH DNA |
| 372 | PaB670100 | VH PRT |
| 373 | PaB670100 | CDR1 PRT |
| 374 | PaB670100 | CDR2 PRT |
| 375 | PaB670100 | CDR3 PRT |
| 376 | PaB670100 | VL DNA |
| 377 | PaB670100 | VL PRT |
| 378 | PaB670100 | CDR1 PRT |
| 379 | PaB670100 | CDR2 PRT |
| 380 | PaB670100 | CDR3 PRT |
| 381 | ZZ1ODO-H01 (PaB670101) | VH DNA |
| 382 | ZZ1ODO-H01 (PaB670101) | VH PRT |
| 383 | ZZ1ODO-H01 (PaB670101) | CDR1 PRT |
| 384 | ZZ1ODO-H01 (PaB670101) | CDR2 PRT |
| 385 | ZZ1ODO-H01 (PaB670101) | CDR3 PRT |
| 386 | ZZ1ODO-H01 (PaB670101) | VL DNA |
| 387 | ZZ1ODO-H01 (PaB670101) | VL PRT |
| 388 | ZZ1ODO-H01 (PaB670101) | CDR1 PRT |
| 389 | ZZ1ODO-H01 (PaB670101) | CDR2 PRT |
| 390 | ZZ1ODO-H01 (PaB670101) | CDR3 PRT |
| 391 | ZZ1PXS-F08 (PaB670102) | VH DNA |
| 392 | ZZ1PXS-F08 (PaB670102) | VH PRT |
| 393 | ZZ1PXS-F08 (PaB670102) | CDR1 PRT |
| 394 | ZZ1PXS-F08 (PaB670102) | CDR2 PRT |
| 395 | ZZ1PXS-F08 (PaB670102) | CDR3 PRT |
| 396 | ZZ1PXS-F08 (PaB670102) | VL DNA |
| 397 | ZZ1PXS-F08 (PaB670102) | VL PRT |
| 398 | ZZ1PXS-F08 (PaB670102) | CDR1 PRT |
| 399 | ZZ1PXS-F08 (PaB670102) | CDR2 PRT |
| 400 | ZZ1PXS-F08 (PaB670102) | CDR3 PRT |
| 401 | ZZ1RCX-C09 (PaB670103) | VH DNA |
| 402 | ZZ1RCX-C09 (PaB670103) | VH PRT |
| 403 | ZZ1RCX-C09 (PaB670103) | CDR1 PRT |
| 404 | ZZ1RCX-C09 (PaB670103) | CDR2 PRT |
| 405 | ZZ1RCX-C09 (PaB670103) | CDR3 PRT |
| 406 | ZZ1RCX-C09 (PaB670103) | VL DNA |
| 407 | ZZ1RCX-C09 (PaB670103) | VL PRT |
| 408 | ZZ1RCX-C09 (PaB670103) | CDR1 PRT |
| 409 | ZZ1RCX-C09 (PaB670103) | CDR2 PRT |
| 410 | ZZ1RCX-C09 (PaB670103) | CDR3 PRT |
| 411 | PaB670104 | VH DNA |
| 412 | PaB670104 | VH PRT |
| 413 | PaB670104 | CDR1 PRT |
| 414 | PaB670104 | CDR2 PRT |
| 415 | PaB670104 | CDR3 PRT |
| 416 | PaB670104 | VL DNA |
| 417 | PaB670104 | VL PRT |
| 418 | PaB670104 | CDR1 PRT |
| 419 | PaB670104 | CDR2 PRT |
| 420 | PaB670104 | CDR3 PRT |
| 421 | ZZ1RD0-D01 (PaB670105) | VH DNA |
| 422 | ZZ1RD0-D01 (PaB670105) | VH PRT |
| 423 | ZZ1RD0-D01 (PaB670105) | CDR1 PRT |
| 424 | ZZ1RD0-D01 (PaB670105) | CDR2 PRT |
| 425 | ZZ1RD0-D01 (PaB670105) | CDR3 PRT |
| 426 | ZZ1RD0-D01 (PaB670105) | VL DNA |
| 427 | ZZ1RD0-D01 (PaB670105) | VL PRT |
| 428 | ZZ1RD0-D01 (PaB670105) | CDR1 PRT |
| 429 | ZZ1RD0-D01 (PaB670105) | CDR2 PRT |
| 430 | ZZ1RD0-D01 (PaB670105) | CDR3 PRT |
| 431 | ZZ1RD0-G02 (PaB670106) | VH DNA |
| 432 | ZZ1RD0-G02 (PaB670106) | VH PRT |
| 433 | ZZ1RD0-G02 (PaB670106) | CDR1 PRT |
| 434 | ZZ1RD0-G02 (PaB670106) | CDR2 PRT |
| 435 | ZZ1RD0-G02 (PaB670106) | CDR3 PRT |
| 436 | ZZ1RD0-G02 (PaB670106) | VL DNA |
| 437 | ZZ1RD0-G02 (PaB670106) | VL PRT |
| 438 | ZZ1RD0-G02 (PaB670106) | CDR1 PRT |
| 439 | ZZ1RD0-G02 (PaB670106) | CDR2 PRT |
| 440 | ZZ1RD0-G02 (PaB670106) | CDR3 PRT |
| 441 | ZZ1RD3-D09 (PaB670107) | VH DNA |
| 442 | ZZ1RD3-D09 (PaB670107) | VH PRT |
| 443 | ZZ1RD3-D09 (PaB670107) | CDR1 PRT |
| 444 | ZZ1RD3-D09 (PaB670107) | CDR2 PRT |
| 445 | ZZ1RD3-D09 (PaB670107) | CDR3 PRT |
| 446 | ZZ1RD3-D09 (PaB670107) | VL DNA |
| 447 | ZZ1RD3-D09 (PaB670107) | VL PRT |
| 448 | ZZ1RD3-D09 (PaB670107) | CDR1 PRT |
| 449 | ZZ1RD3-D09 (PaB670107) | CDR2 PRT |
| 450 | ZZ1RD3-D09 (PaB670107) | CDR3 PRT |
| 451 | ZZ1RD3-H03 (PaB670108) | VH DNA |
| 452 | ZZ1RD3-H03 (PaB670108) | VH PRT |
| 453 | ZZ1RD3-H03 (PaB670108) | CDR1 PRT |
| 454 | ZZ1RD3-H03 (PaB670108) | CDR2 PRT |
| 455 | ZZ1RD3-H03 (PaB670108) | CDR3 PRT |
| 456 | ZZ1RD3-H03 (PaB670108) | VL DNA |
| 457 | ZZ1RD3-H03 (PaB670108) | VL PRT |
| 458 | ZZ1RD3-H03 (PaB670108) | CDR1 PRT |
| 459 | ZZ1RD3-H03 (PaB670108) | CDR2 PRT |
| 460 | ZZ1RD3-H03 (PaB670108) | CDR3 PRT |
| 461 | ZZ1RUC-C01 (PaB670114) | VH DNA |
| 462 | ZZ1RUC-C01 (PaB670114) | VH PRT |
| 463 | ZZ1RUC-C01 (PaB670114) | CDR1 PRT |
| 464 | ZZ1RUC-C01 (PaB670114) | CDR2 PRT |
| 465 | ZZ1RUC-C01 (PaB670114) | CDR3 PRT |
| 466 | ZZ1RUC-C01 (PaB670114) | VL DNA |
| 467 | ZZ1RUC-C01 (PaB670114) | VL PRT |
| 468 | ZZ1RUC-C01 (PaB670114) | CDR1 PRT |
| 469 | ZZ1RUC-C01 (PaB670114) | CDR2 PRT |
| 470 | ZZ1RUC-C01 (PaB670114) | CDR3 PRT |
| 471 | ZZ1RUC-G02 (PaB670115) | VH DNA |
| 472 | ZZ1RUC-G02 (PaB670115) | VH PRT |
| 473 | ZZ1RUC-G02 (PaB670115) | CDR1 PRT |
| 474 | ZZ1RUC-G02 (PaB670115) | CDR2 PRT |
| 475 | ZZ1RUC-G02 (PaB670115) | CDR3 PRT |
| 476 | ZZ1RUC-G02 (PaB670115) | VL DNA |
| 477 | ZZ1RUC-G02 (PaB670115) | VL PRT |
| 478 | ZZ1RUC-G02 (PaB670115) | CDR1 PRT |
| 479 | ZZ1RUC-G02 (PaB670115) | CDR2 PRT |
| 480 | ZZ1RUC-G02 (PaB670115) | CDR3 PRT |
| 481 | ZZ1RUC-B04 (PaB670116) | VH DNA |
| 482 | ZZ1RUC-B04 (PaB670116) | VH PRT |
| 483 | ZZ1RUC-B04 (PaB670116) | CDR1 PRT |
| 484 | ZZ1RUC-B04 (PaB670116) | CDR2 PRT |
| 485 | ZZ1RUC-B04 (PaB670116) | CDR3 PRT |
| 486 | ZZ1RUC-B04 (PaB670116) | VL DNA |
| 487 | ZZ1RUC-B04 (PaB670116) | VL PRT |
| 488 | ZZ1RUC-B04 (PaB670116) | CDR1 PRT |
| 489 | ZZ1RUC-B04 (PaB670116) | CDR2 PRT |
| 490 | ZZ1RUC-B04 (PaB670116) | CDR3 PRT |
| 491 | ZZ1RUC-A06 (PaB670117) | VH DNA |
| 492 | ZZ1RUC-A06 (PaB670117) | VH PRT |
| 493 | ZZ1RUC-A06 (PaB670117) | CDR1 PRT |
| 494 | ZZ1RUC-A06 (PaB670117) | CDR2 PRT |
| 495 | ZZ1RUC-A06 (PaB670117) | CDR3 PRT |
| 496 | ZZ1RUC-A06 (PaB670117) | VL DNA |
| 497 | ZZ1RUC-A06 (PaB670117) | VL PRT |
| 498 | ZZ1RUC-A06 (PaB670117) | CDR1 PRT |
| 499 | ZZ1RUC-A06 (PaB670117) | CDR2 PRT |
| 500 | ZZ1RUC-A06 (PaB670117) | CDR3 PRT |
| 501 | ZZ1RUC-A07 (PaB670118) | VH DNA |
| 502 | ZZ1RUC-A07 (PaB670118) | VH PRT |
| 503 | ZZ1RUC-A07 (PaB670118) | CDR1 PRT |
| 504 | ZZ1RUC-A07 (PaB670118) | CDR2 PRT |
| 505 | ZZ1RUC-A07 (PaB670118) | CDR3 PRT |
| 506 | ZZ1RUC-A07 (PaB670118) | VL DNA |
| 507 | ZZ1RUC-A07 (PaB670118) | VL PRT |
| 508 | ZZ1RUC-A07 (PaB670118) | CDR1 PRT |
| 509 | ZZ1RUC-A07 (PaB670118) | CDR2 PRT |
| 510 | ZZ1RUC-A07 (PaB670118) | CDR3 PRT |
| 511 | ZZ1RUC-G08 (PaB670119) | VH DNA |
| 512 | ZZ1RUC-G08 (PaB670119) | VH PRT |
| 513 | ZZ1RUC-G08 (PaB670119) | CDR1 PRT |
| 514 | ZZ1RUC-G08 (PaB670119) | CDR2 PRT |
| 515 | ZZ1RUC-G08 (PaB670119) | CDR3 PRT |
| 516 | ZZ1RUC-G08 (PaB670119) | VL DNA |
| 517 | ZZ1RUC-G08 (PaB670119) | VL PRT |
| 518 | ZZ1RUC-G08 (PaB670119) | CDR1 PRT |
| 519 | ZZ1RUC-G08 (PaB670119) | CDR2 PRT |
| 520 | ZZ1RUC-G08 (PaB670119) | CDR3 PRT |
| 521 | ZZ1RUC-C11 (PaB670120) | VH DNA |
| 522 | ZZ1RUC-C11 (PaB670120) | VH PRT |
| 523 | ZZ1RUC-C11 (PaB670120) | CDR1 PRT |
| 524 | ZZ1RUC-C11 (PaB670120) | CDR2 PRT |
| 525 | ZZ1RUC-C11 (PaB670120) | CDR3 PRT |
| 526 | ZZ1RUC-C11 (PaB670120) | VL DNA |
| 527 | ZZ1RUC-C11 (PaB670120) | VL PRT |
| 528 | ZZ1RUC-C11 (PaB670120) | CDR1 PRT |
| 529 | ZZ1RUC-C11 (PaB670120) | CDR2 PRT |
| 530 | ZZ1RUC-C11 (PaB670120) | CDR3 PRT |
| 531 | ZZ1RUC-H11 (PaB670121) | VH DNA |
| 532 | ZZ1RUC-H11 (PaB670121) | VH PRT |
| 533 | ZZ1RUC-H11 (PaB670121) | CDR1 PRT |
| 534 | ZZ1RUC-H11 (PaB670121) | CDR2 PRT |
| 535 | ZZ1RUC-H11 (PaB670121) | CDR3 PRT |
| 536 | ZZ1RUC-H11 (PaB670121) | VL DNA |
| 537 | ZZ1RUC-H11 (PaB670121) | VL PRT |
| 538 | ZZ1RUC-H11 (PaB670121) | CDR1 PRT |
| 539 | ZZ1RUC-H11 (PaB670121) | CDR2 PRT |
| 540 | ZZ1RUC-H11 (PaB670121) | CDR3 PRT |
| 541 | ZZ1RUD-A02 (PaB670122) | VH DNA |
| 542 | ZZ1RUD-A02 (PaB670122) | VH PRT |
| 543 | ZZ1RUD-A02 (PaB670122) | CDR1 PRT |
| 544 | ZZ1RUD-A02 (PaB670122) | CDR2 PRT |
| 545 | ZZ1RUD-A02 (PaB670122) | CDR3 PRT |
| 546 | ZZ1RUD-A02 (PaB670122) | VL DNA |
| 547 | ZZ1RUD-A02 (PaB670122) | VL PRT |
| 548 | ZZ1RUD-A02 (PaB670122) | CDR1 PRT |
| 549 | ZZ1RUD-A02 (PaB670122) | CDR2 PRT |
| 550 | ZZ1RUD-A02 (PaB670122) | CDR3 PRT |
| 551 | ZZ1RUD-H03 (PaB670123) | VH DNA |
| 552 | ZZ1RUD-H03 (PaB670123) | VH PRT |
| 553 | ZZ1RUD-H03 (PaB670123) | CDR1 PRT |
| 554 | ZZ1RUD-H03 (PaB670123) | CDR2 PRT |
| 555 | ZZ1RUD-H03 (PaB670123) | CDR3 PRT |
| 556 | ZZ1RUD-H03 (PaB670123) | VL DNA |
| 557 | ZZ1RUD-H03 (PaB670123) | VL PRT |
| 558 | ZZ1RUD-H03 (PaB670123) | CDR1 PRT |
| 559 | ZZ1RUD-H03 (PaB670123) | CDR2 PRT |
| 560 | ZZ1RUD-H03 (PaB670123) | CDR3 PRT |
| 561 | ZZ1RUD-H06 (PaB670125) | VH DNA |
| 562 | ZZ1RUD-H06 (PaB670125) | VH PRT |
| 563 | ZZ1RUD-H06 (PaB670125) | CDR1 PRT |
| 564 | ZZ1RUD-H06 (PaB670125) | CDR2 PRT |
| 565 | ZZ1RUD-H06 (PaB670125) | CDR3 PRT |
| 566 | ZZ1RUD-H06 (PaB670125) | VL DNA |
| 567 | ZZ1RUD-H06 (PaB670125) | VL PRT |
| 568 | ZZ1RUD-H06 (PaB670125) | CDR1 PRT |
| 569 | ZZ1RUD-H06 (PaB670125) | CDR2 PRT |
| 570 | ZZ1RUD-H06 (PaB670125) | CDR3 PRT |
| 571 | ZZ1RUD-B08 (PaB670126) | VH DNA |
| 572 | ZZ1RUD-B08 (PaB670126) | VH PRT |
| 573 | ZZ1RUD-B08 (PaB670126) | CDR1 PRT |
| 574 | ZZ1RUD-B08 (PaB670126) | CDR2 PRT |
| 575 | ZZ1RUD-B08 (PaB670126) | CDR3 PRT |
| 576 | ZZ1RUD-B08 (PaB670126) | VL DNA |
| 577 | ZZ1RUD-B08 (PaB670126) | VL PRT |
| 578 | ZZ1RUD-B08 (PaB670126) | CDR1 PRT |
| 579 | ZZ1RUD-B08 (PaB670126) | CDR2 PRT |
| 580 | ZZ1RUD-B08 (PaB670126) | CDR3 PRT |
| 581 | ZZ1RUD-H10 (PaB670127) | VH DNA |
| 582 | ZZ1RUD-H10 (PaB670127) | VH PRT |
| 583 | ZZ1RUD-H10 (PaB670127) | CDR1 PRT |
| 584 | ZZ1RUD-H10 (PaB670127) | CDR2 PRT |
| 585 | ZZ1RUD-H10 (PaB670127) | CDR3 PRT |
| 586 | ZZ1RUD-H10 (PaB670127) | VL DNA |
| 587 | ZZ1RUD-H10 (PaB670127) | VL PRT |
| 588 | ZZ1RUD-H10 (PaB670127) | CDR1 PRT |
| 589 | ZZ1RUD-H10 (PaB670127) | CDR2 PRT |
| 590 | ZZ1RUD-H10 (PaB670127) | CDR3 PRT |
| 591 | ZZ1RUE−A01 (PaB670128) | VH DNA |
| 592 | ZZ1RUE−A01 (PaB670128) | VH PRT |
| 593 | ZZ1RUE−A01 (PaB670128) | CDR1 PRT |
| 594 | ZZ1RUE−A01 (PaB670128) | CDR2 PRT |
| 595 | ZZ1RUE−A01 (PaB670128) | CDR3 PRT |
| 596 | ZZ1RUE−A01 (PaB670128) | VL DNA |
| 597 | ZZ1RUE−A01 (PaB670128) | VL PRT |
| 598 | ZZ1RUE−A01 (PaB670128) | CDR1 PRT |
| 599 | ZZ1RUE−A01 (PaB670128) | CDR2 PRT |
| 600 | ZZ1RUE−A01 (PaB670128) | CDR3 PRT |
| 601 | ZZ1RUF-A01 (PaB670136) | VH DNA |
| 602 | ZZ1RUF-A01 (PaB670136) | VH PRT |
| 603 | ZZ1RUF-A01 (PaB670136) | CDR1 PRT |
| 604 | ZZ1RUF-A01 (PaB670136) | CDR2 PRT |
| 605 | ZZ1RUF-A01 (PaB670136) | CDR3 PRT |
| 606 | ZZ1RUF-A01 (PaB670136) | VL DNA |
| 607 | ZZ1RUF-A01 (PaB670136) | VL PRT |
| 608 | ZZ1RUF-A01 (PaB670136) | CDR1 PRT |
| 609 | ZZ1RUF-A01 (PaB670136) | CDR2 PRT |
| 610 | ZZ1RUF-A01 (PaB670136) | CDR3 PRT |
| 611 | ZZ1RUF-B06 (PaB670137) | VH DNA |
| 612 | ZZ1RUF-B06 (PaB670137) | VH PRT |
| 613 | ZZ1RUF-B06 (PaB670137) | CDR1 PRT |
| 614 | ZZ1RUF-B06 (PaB670137) | CDR2 PRT |
| 615 | ZZ1RUF-B06 (PaB670137) | CDR3 PRT |
| 616 | ZZ1RUF-B06 (PaB670137) | VL DNA |
| 617 | ZZ1RUF-B06 (PaB670137) | VL PRT |
| 618 | ZZ1RUF-B06 (PaB670137) | CDR1 PRT |
| 619 | ZZ1RUF-B06 (PaB670137) | CDR2 PRT |
| 620 | ZZ1RUF-B06 (PaB670137) | CDR3 PRT |
| 621 | PaB670141 | VH DNA |
| 622 | PaB670141 | VH PRT |
| 623 | PaB670141 | CDR1 PRT |
| 624 | PaB670141 | CDR2 PRT |
| 625 | PaB670141 | CDR3 PRT |
| 626 | PaB670141 | VL DNA |
| 627 | PaB670141 | VL PRT |
| 628 | PaB670141 | CDR1 PRT |
| 629 | PaB670141 | CDR2 PRT |
| 630 | PaB670141 | CDR3 PRT |
| 631 | PaB670142 | VH DNA |
| 632 | PaB670142 | VH PRT |
| 633 | PaB670142 | CDR1 PRT |
| 634 | PaB670142 | CDR2 PRT |
| 635 | PaB670142 | CDR3 PRT |
| 636 | PaB670142 | VL DNA |
| 637 | PaB670142 | VL PRT |
| 638 | PaB670142 | CDR1 PRT |
| 639 | PaB670142 | CDR2 PRT |
| 640 | PaB670142 | CDR3 PRT |
| 641 | PaB670143 | VH DNA |
| 642 | PaB670143 | VH PRT |
| 643 | PaB670143 | CDR1 PRT |
| 644 | PaB670143 | CDR2 PRT |
| 645 | PaB670143 | CDR3 PRT |
| 646 | PaB670143 | VL DNA |
| 647 | PaB670143 | VL PRT |
| 648 | PaB670143 | CDR1 PRT |
| 649 | PaB670143 | CDR2 PRT |
| 650 | PaB670143 | CDR3 PRT |
| 651 | PaB670144 | VH DNA |
| 652 | PaB670144 | VH PRT |
| 653 | PaB670144 | CDR1 PRT |
| 654 | PaB670144 | CDR2 PRT |
| 655 | PaB670144 | CDR3 PRT |
| 656 | PaB670144 | VL DNA |
| 657 | PaB670144 | VL PRT |
| 658 | PaB670144 | CDR1 PRT |
| 659 | PaB670144 | CDR2 PRT |
| 660 | PaB670144 | CDR3 PRT |
| 661 | PaB670146 | VH DNA |
| 662 | PaB670146 | VH PRT |
| 663 | PaB670146 | CDR1 PRT |
| 664 | PaB670146 | CDR2 PRT |
| 665 | PaB670146 | CDR3 PRT |
| 666 | PaB670146 | VL DNA |
| 667 | PaB670146 | VL PRT |
| 668 | PaB670146 | CDR1 PRT |
| 669 | PaB670146 | CDR2 PRT |
| 670 | PaB670146 | CDR3 PRT |
| 671 | PaB670148 | VH DNA |
| 672 | PaB670148 | VH PRT |
| 673 | PaB670148 | CDR1 PRT |
| 674 | PaB670148 | CDR2 PRT |
| 675 | PaB670148 | CDR3 PRT |
| 676 | PaB670148 | VL DNA |
| 677 | PaB670148 | VL PRT |
| 678 | PaB670148 | CDR1 PRT |
| 679 | PaB670148 | CDR2 PRT |
| 680 | PaB670148 | CDR3 PRT |
| 681 | PaB670149 | VH DNA |
| 682 | PaB670149 | VH PRT |
| 683 | PaB670149 | CDR1 PRT |
| 684 | PaB670149 | CDR2 PRT |
| 685 | PaB670149 | CDR3 PRT |
| 686 | PaB670149 | VL DNA |
| 687 | PaB670149 | VL PRT |
| 688 | PaB670149 | CDR1 PRT |
| 689 | PaB670149 | CDR2 PRT |
| 690 | PaB670149 | CDR3 PRT |
| 691 | PaB670151 | VH DNA |
| 692 | PaB670151 | VH PRT |
| 693 | PaB670151 | CDR1 PRT |
| 694 | PaB670151 | CDR2 PRT |
| 695 | PaB670151 | CDR3 PRT |
| 696 | PaB670151 | VL DNA |
| 697 | PaB670151 | VL PRT |
| 698 | PaB670151 | CDR1 PRT |
| 699 | PaB670151 | CDR2 PRT |
| 700 | PaB670151 | CDR3 PRT |
| 701 | PaB670152 | VH DNA |
| 702 | PaB670152 | VH PRT |
| 703 | PaB670152 | CDR1 PRT |
| 704 | PaB670152 | CDR2 PRT |
| 705 | PaB670152 | CDR3 PRT |
| 706 | PaB670152 | VL DNA |
| 707 | PaB670152 | VL PRT |
| 708 | PaB670152 | CDR1 PRT |
| 709 | PaB670152 | CDR2 PRT |
| 710 | PaB670152 | CDR3 PRT |
| 711 | PaB670153 | VH DNA |
| 712 | PaB670153 | VH PRT |
| 713 | PaB670153 | CDR1 PRT |
| 714 | PaB670153 | CDR2 PRT |
| 715 | PaB670153 | CDR3 PRT |
| 716 | PaB670153 | VL DNA |
| 717 | PaB670153 | VL PRT |
| 718 | PaB670153 | CDR1 PRT |
| 719 | PaB670153 | CDR2 PRT |
| 720 | PaB670153 | CDR3 PRT |
| 721 | PaB670156 | VH DNA |
| 722 | PaB670156 | VH PRT |
| 723 | PaB670156 | CDR1 PRT |
| 724 | PaB670156 | CDR2 PRT |
| 725 | PaB670156 | CDR3 PRT |
| 726 | PaB670156 | VL DNA |
| 727 | PaB670156 | VL PRT |
| 728 | PaB670156 | CDR1 PRT |
| 729 | PaB670156 | CDR2 PRT |
| 730 | PaB670156 | CDR3 PRT |
| 731 | PaB670157 | VH DNA |
| 732 | PaB670157 | VH PRT |
| 733 | PaB670157 | CDR1 PRT |
| 734 | PaB670157 | CDR2 PRT |
| 735 | PaB670157 | CDR3 PRT |
| 736 | PaB670157 | VL DNA |
| 737 | PaB670157 | VL PRT |
| 738 | PaB670157 | CDR1 PRT |
| 739 | PaB670157 | CDR2 PRT |
| 740 | PaB670157 | CDR3 PRT |
| 741 | PaB670158 | VH DNA |
| 742 | PaB670158 | VH PRT |
| 743 | PaB670158 | CDR1 PRT |
| 744 | PaB670158 | CDR2 PRT |
| 745 | PaB670158 | CDR3 PRT |
| 746 | PaB670158 | VL DNA |
| 747 | PaB670158 | VL PRT |
| 748 | PaB670158 | CDR1 PRT |
| 749 | PaB670158 | CDR2 PRT |
| 750 | PaB670158 | CDR3 PRT |
| 751 | PaB670159 | VH DNA |
| 752 | PaB670159 | VH PRT |
| 753 | PaB670159 | CDR1 PRT |
| 754 | PaB670159 | CDR2 PRT |
| 755 | PaB670159 | CDR3 PRT |
| 756 | PaB670159 | VL DNA |
| 757 | PaB670159 | VL PRT |
| 758 | PaB670159 | CDR1 PRT |
| 759 | PaB670159 | CDR2 PRT |
| 760 | PaB670159 | CDR3 PRT |
| 761 | PaB670160 | VH DNA |
| 762 | PaB670160 | VH PRT |
| 763 | PaB670160 | CDR1 PRT |
| 764 | PaB670160 | CDR2 PRT |
| 765 | PaB670160 | CDR3 PRT |
| 766 | PaB670160 | VL DNA |
| 767 | PaB670160 | VL PRT |
| 768 | PaB670160 | CDR1 PRT |
| 769 | PaB670160 | CDR2 PRT |
| 770 | PaB670160 | CDR3 PRT |
| 771 | PaB670161 | VH DNA |
| 772 | PaB670161 | VH PRT |
| 773 | PaB670161 | CDR1 PRT |
| 774 | PaB670161 | CDR2 PRT |
| 775 | PaB670161 | CDR3 PRT |
| 776 | PaB670161 | VL DNA |
| 777 | PaB670161 | VL PRT |
| 778 | PaB670161 | CDR1 PRT |
| 779 | PaB670161 | CDR2 PRT |
| 780 | PaB670161 | CDR3 PRT |
| 781 | PaB670162 | VH DNA |
| 782 | PaB670162 | VH PRT |
| 783 | PaB670162 | CDR1 PRT |
| 784 | PaB670162 | CDR2 PRT |
| 785 | PaB670162 | CDR3 PRT |
| 786 | PaB670162 | VL DNA |
| 787 | PaB670162 | VL PRT |
| 788 | PaB670162 | CDR1 PRT |
| 789 | PaB670162 | CDR2 PRT |
| 790 | PaB670162 | CDR3 PRT |
| 791 | PaB670163 | VH DNA |
| 792 | PaB670163 | VH PRT |
| 793 | PaB670163 | CDR1 PRT |
| 794 | PaB670163 | CDR2 PRT |
| 795 | PaB670163 | CDR3 PRT |
| 796 | PaB670163 | VL DNA |
| 797 | PaB670163 | VL PRT |
| 798 | PaB670163 | CDR1 PRT |
| 799 | PaB670163 | CDR2 PRT |
| 800 | PaB670163 | CDR3 PRT |

PRT = amino acid sequence

Human PAR2 Preproprotein
(GenBank Accession No. NP_005233.3)
SEQ ID NO: 801
MRSPSAAWLLGAAILLAASLSCSGTIQGTNRSSKGRSLIGKVDGTSHVT
GKGVTVETVFSVDEFSASVLTGKLTTVFLPIVYTIVFVVGLPSNGMALW
VFLFRTKKKHPAVIYMANLALADLLSVIWFPLKIAYHIHGNNWIYGEAL
CNVLIGFFYGNMYCSILFMTCLSVQRYWVIVNPMGHSRKKANIAIGISL
AIWLLILLVTIPLYVVKQTIFIPALNITTCHDVLPEQLLVGDMFNYFLS
LAIGVFLFPAFLTASAYVLMIRMLRSSAMDENSEKKRKRAIKLIVTVLA
MYLICFTPSNLLLVVHYFLIKSQGQSHVYALYIVALCLSTLNSCIDPFV
YYFVSHDFRDHAKNALLCRSVRTVKQMQVSLTSKKHSRKSSSYSSSSTT
VKTSY
Human PAR2 Tethered Ligand
SEQ ID NO: 802
SLIGKVDGTSHVTGKGVTVETVFSVDEFSASVLTGKLTT
Exemplary VH Framework Region 1
SEQ ID NO: 803
EVQLLESGGGLVQPGGSLRLSCAASGFTFS
Exemplary VH Framework Region 2
SEQ ID NO: 804
WVRQAPGKGLEWVS
Exemplary VH Framework Region 3
SEQ ID NO: 805
RFTISRDNSKNTLYLQMNSLRAEDTAVYYCAR
Exemplary VH Framework Region 4
SEQ ID NO: 806
WGQGTLVTVSS
Exemplary VL Framework Region 1
SEQ ID NO: 807
SIELTQPPSVSVSPGQTASITC
Exemplary VL Framework Region 2
SEQ ID NO: 808
WYQQKPGQSPVLVIY
Exemplary VL Framework Region 3
SEQ ID NO: 809
GIPERFSGSNSGNTATLTISGTQAMDEADYYC
Exemplary VL Framework Region 4
SEQ ID NO: 810
FGGGTKLTVL
Exemplary VH CDR2
SEQ ID NO: 811
TISYSGSHISYHDSVHH
Exemplary VH CDR2
SEQ ID NO: 812
TISYHGSLISYHDSVHH
Exemplary VH CDR2
SEQ ID NO: 813
TISYHGSHISYADSVHH
Exemplary VH CDR2
SEQ ID NO: 814
TISYHGSHISYHDSVKH
Exemplary VH CDR2
SEQ ID NO: 815
TISYHGSHISYHDSVHG
Exemplary VH CDR2
SEQ ID NO: 816
TISYHGSLISYADSVKG
Exemplary VH CDR2
SEQ ID NO: 817
TISYSGSHISYADSVKG
Exemplary VH CDR2
SEQ ID NO: 818
TISYHGSHISYADSVKG
Exemplary VH CDR3
SEQ ID NO: 819
IHNDPMDV
Exemplary VH CDR3
SEQ ID NO: 820
INHDPMDV
Exemplary VH
SEQ ID NO: 821
EVQLLESGGGLVQPGGSLRLSCAASGFTFSSYAMNWVRQAPGKGLEWVS
TISYHGSHISYHDSVHHRFTISRDNSKNTLYLQMNSLRAEDTAVYYCAR
IHHDPMDVWGQGTLVTVSS
Exemplary VH
SEQ ID NO: 822
EVQLLESGGGLVQPGGSLRLSCAASGFTFSSYAMNWVRQAPGKGLEWVS
TISYSGSHISYHDSVHHRFTISRDNSKNTLYLQMNSLRAEDTAVYYCAR
IHHDPMDVWGQGTLVTVSS
Exemplary VH
SEQ ID NO: 823
EVQLLESGGGLVQPGGSLRLSCAASGFTFSSYAMNWVRQAPGKGLEWVS
TISYHGSLISYHDSVHHRFTISRDNSKNTLYLQMNSLRAEDTAVYYCAR
IHHDPMDVWGQGTLVTVSS
Exemplary VH
SEQ ID NO: 824
EVQLLESGGGLVQPGGSLRLSCAASGFTFSSYAMNWVRQAPGKGLEWVS
TISYHGSHISYADSVHHRFTISRDNSKNTLYLQMNSLRAEDTAVYYCAR
IHHDPMDVWGQGTLVTVSS
Exemplary VH
SEQ ID NO: 825
EVQLLESGGGLVQPGGSLRLSCAASGFTFSSYAMNWVRQAPGKGLEWVS
TISYHGSHISYHDSVKHRFTISRDNSKNTLYLQMNSLRAEDTAVYYCAR
IHHDPMDVWGQGTLVTVSS
Exemplary VH
SEQ ID NO: 826
EVQLLESGGGLVQPGGSLRLSCAASGFTFSSYAMNWVRQAPGKGLEWVS
TISYHGSHISYHDSVHGRFTISRDNSKNTLYLQMNSLRAEDTAVYYCAR
IHHDPMDVWGQGTLVTVSS
Exemplary VH
SEQ ID NO: 827
EVQLLESGGGLVQPGGSLRLSCAASGFTFSSYAMNWVRQAPGKGLEWVS
TISYHGSHISYHDSVHHRFTISRDNSKNTLYLQMNSLRAEDTAVYYCAR
INHDPMDVWGQGTLVTVSS
Exemplary VH
SEQ ID NO: 828
EVQLLESGGGLVQPGGSLRLSCAASGFTFSSYAMNWVRQAPGKGLEWVS
TISYHGSHISYHDSVHHRFTISRDNSKNTLYLQMNSLRAEDTAVYYCAR
IHNDPMDVWGQGTLVTVSS
Exemplary VH
SEQ ID NO: 829
EVQLLESGGGLVQPGGSLRLSCAASGFTFSSYAMNWVRQAPGKGLEWVS
TISYHGSLISYADSVKGRFTISRDNSKNTLYLQMNSLRAEDTAVYYCAR
IHHDPMDVWGQGTLVTVSS
Exemplary VH
SEQ ID NO: 830
EVQLLESGGGLVQPGGSLRLSCAASGFTFSSYAMNWVRQAPGKGLEWVS
TISYSGSHISYADSVKGRFTISRDNSKNTLYLQMNSLRAEDTAVYYCAR
IHHDPMDVWGQGTLVTVSS
Exemplary VH
SEQ ID NO: 831
EVQLLESGGGLVQPGGSLRLSCAASGFTFSSYAMNWVRQAPGKGLEWVS
TISYHGSHISYADSVKGRFTISRDNSKNTLYLQMNSLRAEDTAVYYCAR
IHHDPMDVWGQGTLVTVSS

### INCORPORATION BY REFERENCE

All publications and patents mentioned herein are hereby incorporated by reference in their entirety as if each individual publication or patent was specifically and individually indicated to be incorporated by reference.

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

What is claimed is:

1. An antibody or antigen-binding fragment thereof comprising a heavy chain variable domain (VH) and a light chain variable domain (VL), wherein the VH comprises:
i) a VH-CDR1 having the amino acid sequence of SEQ ID NO: 3, but wherein 1, 2, 3, 4, or 5 amino acid substitutions, deletions or insertions are optionally present in the sequence of SEQ ID NO: 3;
ii) a VH-CDR2 having the amino acid sequence of SEQ ID NO: 4, but wherein 1, 2, 3, 4, or 5 amino acid substitutions, deletions or insertions are optionally present in the sequence of SEQ ID NO: 4; and
iii) a VH-CDR3 having the amino acid sequence of SEQ ID NO: 5, but wherein 1, 2, 3, 4, or 5 amino acid substitutions, deletions or insertions are optionally present in the sequence of SEQ ID NO: 5;
and wherein the VL comprises:
i) a VL-CDR1 having the amino acid sequence of SEQ ID NO: 8; but wherein 1, 2, 3, 4, or 5 amino acid substitutions, deletions or insertions are optionally present in the sequence of SEQ ID NO: 8;
ii) a VL-CDR2 having the amino acid sequence of SEQ ID NO: 9, but wherein 1, 2, 3, 4, or 5 amino acid substitutions, deletions or insertions are optionally present in the sequence of SEQ ID NO: 9; and
iii) a VL-CDR3 having the amino acid sequence of SEQ ID NO: 10; but wherein 1, 2, 3, 4, or 5 amino acid substitutions, deletions or insertions are optionally present in the sequence of SEQ ID NO: 10;
wherein the amino acid substitutions, deletions or insertions reduce the binding affinity of the antibody or antigen-binding fragment thereof for human PAR2 by no more than 1000, 800, 700, 500, 400, 300, 200, 100, 50 or 10-fold as compared to an antibody or antigen-binding fragment having a VH with an amino acid sequence of SEQ ID NO: 2 and VL with an amino acid sequence of SEQ ID NO: 7 when tested at a pH of 7.4 in a PAR2 binding assay.

2. An antibody or antigen-binding fragment thereof comprising a heavy chain variable domain (VH) and a light chain variable domain (VL), wherein the VH comprises:
i) a VH-CDR1 having the amino acid sequence of SEQ ID NO: 3;
ii) a VH-CDR2 having the amino acid sequence of SEQ ID NO: 4, but wherein a histidine is optionally present at any one or more of the amino acid positions corresponding to positions 1-17 of SEQ ID NO: 4; and
iii) a VH-CDR3 having the amino acid sequence of SEQ ID NO: 5, but wherein a histidine is optionally present at any one or more of the amino acid positions corresponding to positions 1-8 of SEQ ID NO: 5;
and wherein the VL comprises:
i) a VL-CDR1 having the amino acid sequence of SEQ ID NO: 8;
ii) a VL-CDR2 having the amino acid sequence of SEQ ID NO: 9; and
iii) a VL-CDR3 having the amino acid sequence of SEQ ID NO: 10; but wherein a histidine is optionally present at any one or more of the amino acid positions corresponding to positions 1-14 of SEQ ID NO: 10.

3. The antibody or antigen-binding fragment thereof of claim 2, wherein the VH comprises:
i) a VH-CDR1 having the amino acid sequence of SEQ ID NO: 3,
ii) a VH-CDR2 having the amino acid sequence of SEQ ID NO: 4,
iii) a VH-CDR3 having the amino acid sequence of SEQ ID NO: 5,
and wherein the VL comprises:
i) a VL-CDR1 having the amino acid sequence of SEQ ID NO: 8,
ii) a VL-CDR2 having the amino acid sequence of SEQ ID NO: 9,
iii) a VL-CDR3 having the amino acid sequence of SEQ ID NO: 10.

4. The antibody or antigen-binding fragment thereof of claim 2, wherein the VH comprises:
i) a VH-CDR1 having the amino acid sequence of SEQ ID NO: 13,
ii) a VH-CDR2 having the amino acid sequence of SEQ ID NO: 14,
iii) a VH-CDR3 having the amino acid sequence of SEQ ID NO: 15,
and wherein the VL comprises:
i) a VL-CDR1 having the amino acid sequence of SEQ ID NO: 18,
ii) a VL-CDR2 having the amino acid sequence of SEQ ID NO: 19,
iii) a VL-CDR3 having the amino acid sequence of SEQ ID NO: 20.

5. The antibody or antigen-binding fragment thereof of claim 2, wherein a histidine is present at any one or more of the amino acid positions corresponding to:
a) positions 5, 7, 8, 12, 15, 16 or 17 of SEQ ID NO: 4;
b) positions 2 or 3 of SEQ ID NO: 5; and/or
c) positions 1, 5, 6 or 14 of SEQ ID NO: 10.

6. The antibody or antigen-binding fragment thereof of claim 2, wherein a histidine is present at the amino acid positions corresponding to positions 5 and 8 of SEQ ID NO: 4.

7. The antibody or antigen-binding fragment thereof of claim 6, wherein a histidine is present at the amino acid positions corresponding to positions 2 and 3 of SEQ ID NO: 5.

8. The antibody or antigen-binding fragment thereof of claim 2, wherein:
a) the VH-CDR2 comprises an amino acid sequence selected from the group consisting of: SEQ ID NOs: 4, 14, 24, 34, 44, 54, 64, 74, 84, 94, 104, 114, 124, 134, 144, 154, 164, 174, 184, 194, 204, 214, 224, 234, 244, 254, 264, 274, 284, 294, 304, 314, 324, 334, 344, 354, 364, 374, 384, 394, 404, 414, 424, 434, 444, 454, 464, 474, 484, 494, 504, 514, 524, 534, 544, 554, 564, 574, 584, 594, 604, 614, 624, 634, 644, 654, 664, 674, 684, 694, 704, 714, 724, 734, 744, 754, 764, 774, 784, 794, and 811-818;
b) the VH-CDR3 comprises an amino acid sequence selected from the group consisting of: SEQ ID NOs: 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, 645, 655, 665, 675, 685, 695, 705, 715, 725, 735, 745, 755, 765, 775, 785, 795, and 819-820; and/or
c) the VL-CDR3 comprises an amino acid sequence selected from the group consisting of: SEQ ID NOs: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790 and 800.

9. The antibody or antigen-binding fragment thereof of claim 2, wherein the VH-CDR2 comprises an amino acid sequence corresponding to SEQ ID NO: 14; wherein the VH-CDR3 comprises an amino acid sequence corresponding to SEQ ID NO: 15, and wherein the VL-CDR3 comprises an amino acid sequence corresponding to SEQ ID NO: 20.

10. The antibody or antigen-binding fragment thereof of claim 2, wherein the VH-CDR2 comprises an amino acid sequence corresponding to SEQ ID NO: 818; wherein the VH-CDR3 comprises an amino acid sequence corresponding to SEQ ID NO: 15, and wherein the VL-CDR3 comprises an amino acid sequence corresponding to SEQ ID NO: 10 or 20.

11. The antibody or antigen-binding fragment thereof of claim 2, wherein:
a) the VH comprises an amino acid sequence that is at least 80%, 85%, 90%, 92%, 93%, 95%, 97%, 98%, 99% or 100% identical to a sequence selected from the group consisting of: SEQ ID NOs: 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, 362, 372, 382, 392, 402, 412, 422, 432, 442, 452, 462, 472, 482, 492, 502, 512, 522, 532, 542, 552, 562, 572, 582, 592, 602, 612, 622, 632, 642, 652, 662, 672, 682, 692, 702, 712, 722, 732, 742, 752, 762, 772, 782, 792, and 821-831; and/or
b) the VL comprises an amino acid sequence that is at least 80%, 85%, 90%, 92%, 93%, 95%, 97%, 98%, 99% or 100% identical to a sequence selected from the group consisting of: SEQ ID NOs: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, 367, 377, 387, 397, 407, 417, 427, 437, 447, 457, 467, 477, 487, 497, 507, 517, 527, 537, 547, 557, 567, 577, 587, 597, 607, 617, 627, 637, 647, 657, 667, 677, 687, 697, 707, 717, 727, 737, 747, 757, 767, 777, 787, and 797.

12. The antibody or antigen-binding fragment thereof of claim 2, wherein the VH comprises an amino acid sequence corresponding to SEQ ID NO: 831 and wherein the VL comprises an amino acid sequence corresponding to SEQ ID NO: 7 or 17.

13. The antibody or antigen-binding fragment thereof of claim 2, wherein the VH comprises an amino acid sequence that is at least 80%, 85%, 90%, 92%, 93%, 95%, 97%, 99% or 100% identical to SEQ ID NO: 12 and wherein the VL comprises an amino acid sequence that is at least 80%, 85%, 90%, 92%, 93%, 95%, 97%, 99% or 100% identical to SEQ ID NO: 17.

14. The antibody or antigen-binding fragment of claim 2, wherein the antigen-binding fragment is an scFv or a Fab′.

15. The antibody or antigen-binding fragment of claim 2, wherein the antibody is a monoclonal antibody.

16. The antibody or antigen-binding fragment of claim 2, wherein the antibody or antigen-binding fragment prevents trypsin, tryptase and/or matriptase from interacting with PAR2.

17. The antibody or antigen-binding fragment of claim 2, wherein the antibody or antigen-binding fragment binds to PAR2 with greater affinity at a pH of 7.4 than at a pH of 6.0.

18. A composition comprising a pharmaceutically acceptable carrier and the antibody or antigen-binding fragment of claim 2.

19. A kit comprising the antibody or antigen-binding fragment of claim 2.

20. A method for treating pain in a subject in need thereof, comprising administering to the subject a pharmaceutically effective amount of the antibody or antigen-binding fragment of claim 2.


SEQUENCE LISTING (841 sequences)

SEQ ID NO: 1 - DNA, 351 bp, Homo sapiens
  source 1..351: mol_type = other DNA
gaggtgcagctgctcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 2 - protein, 117 aa, Homo sapiens
EVQLLESGGGLVQPGGSLRLSCAASGFTFSSYAMNWVRQAPGKGLEWVSTISYSGSLISYADSVKGRFTISRDNSKNTLYLQMNSLRAEDTAVYYCARINNDPMDVWGQGTLVTVSS

SEQ ID NO: 3 - protein, 5 aa, Homo sapiens
SYAMN

SEQ ID NO: 4 - protein, 17 aa, Homo sapiens
TISYSGSLISYADSVKG

SEQ ID NO: 5 - protein, 8 aa, Homo sapiens
INNDPMDV

SEQ ID NO: 6 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggccatggacgaggccgactactactgccagacctgggacgggaacccgacgacgggcgagaccaacgtgttcggcggagggaccaagttaaccgtccta

SEQ ID NO: 7 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQTWDGNPTTGETNVFGGGTKLTVL

SEQ ID NO: 8 - protein, 11 aa, Homo sapiens
SGDNLGKKYVQ

SEQ ID NO: 9 - protein, 7 aa, Homo sapiens
DDSNRPS

SEQ ID NO: 10 - protein, 14 aa, Homo sapiens
QTWDGNPTTGETNV

SEQ ID NO: 11 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgcatcacaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 12 - protein, 117 aa, Homo sapiens
EVQLLESGGGLVQPGGSLRLSCAASGFTFSSYAMNWVRQAPGKGLEWVSTISYHGSHISYHDSVHHRFTISRDNSKNTLYLQMNSLRAEDTAVYYCARIHHDPMDVWGQGTLVTVSS

SEQ ID NO: 13 - protein, 5 aa, Homo sapiens
SYAMN

SEQ ID NO: 14 - protein, 17 aa, Homo sapiens
TISYHGSHISYHDSVHH

SEQ ID NO: 15 - protein, 8 aa, Homo sapiens
IHHDPMDV

SEQ ID NO: 16 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagacctgggacgggaacccgacgacgggcgagaccaacgtgttcggcggagggaccaagttaaccgtccta

SEQ ID NO: 17 - protein, 111 aa, Homo sapiens; identical to SEQ ID NO: 7

SEQ ID NO: 18 - protein, 11 aa, Homo sapiens
SGDNLGKKYVQ

SEQ ID NO: 19 - protein, 7 aa, Homo sapiens
DDSNRPS

SEQ ID NO: 20 - protein, 14 aa, Homo sapiens
QTWDGNPTTGETNV

SEQ ID NO: 21 - DNA, 351 bp, Homo sapiens
  source 1..351: mol_type = other DNA
gaggtgcagctgctcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatccactacgccgacagcgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 22 - protein, 117 aa, Homo sapiens
EVQLLESGGGLVQPGGSLRLSCAASGFTFSSYAMNWVRQAPGKGLEWVSTISYSGSLIHYADSVKGRFTISRDNSKNTLYLQMNSLRAEDTAVYYCARINNDPMDVWGQGTLVTVSS

SEQ ID NO: 23 - protein, 5 aa, Homo sapiens
SYAMN

SEQ ID NO: 24 - protein, 17 aa, Homo sapiens
TISYSGSLIHYADSVKG

SEQ ID NO: 25 - protein, 8 aa, Homo sapiens
INNDPMDV

SEQ ID NO: 26 - DNA, 333 bp, Homo sapiens; identical to SEQ ID NO: 6
  source 1..333: mol_type = other DNA

SEQ ID NO: 27 - protein, 111 aa, Homo sapiens; identical to SEQ ID NO: 7

SEQ ID NO: 28 - protein, 11 aa, Homo sapiens
SGDNLGKKYVQ

SEQ ID NO: 29 - protein, 7 aa, Homo sapiens
DDSNRPS

SEQ ID NO: 30 - protein, 14 aa, Homo sapiens
QTWDGNPTTGETNV

SEQ ID NO: 31 - DNA, 351 bp, Homo sapiens
  source 1..351: mol_type = other DNA
gaggtgcagctgctcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 32 - protein, 117 aa, Homo sapiens
EVQLLESGGGLVQPGGSLRLSCAASGFTFSSYAMNWVRQAPGKGLEWVSTISYSGSLISYADSVKGRFTISRDNSKNTLYLQMNSLRAEDTAVYYCARINHDPMDVWGQGTLVTVSS

SEQ ID NO: 33 - protein, 5 aa, Homo sapiens
SYAMN

SEQ ID NO: 34 - protein, 17 aa, Homo sapiens
TISYSGSLISYADSVKG

SEQ ID NO: 35 - protein, 8 aa, Homo sapiens
INHDPMDV

SEQ ID NO: 36 - DNA, 333 bp, Homo sapiens; identical to SEQ ID NO: 6
  source 1..333: mol_type = other DNA

SEQ ID NO: 37 - protein, 111 aa, Homo sapiens; identical to SEQ ID NO: 7

SEQ ID NO: 38 - protein, 11 aa, Homo sapiens
SGDNLGKKYVQ

SEQ ID NO: 39 - protein, 7 aa, Homo sapiens
DDSNRPS

SEQ ID NO: 40 - protein, 14 aa, Homo sapiens
QTWDGNPTTGETNV

SEQ ID NO: 41 - DNA, 351 bp, Homo sapiens; identical to SEQ ID NO: 1
  source 1..351: mol_type = other DNA

SEQ ID NO: 42 - protein, 117 aa, Homo sapiens; identical to SEQ ID NO: 2

SEQ ID NO: 43 - protein, 5 aa, Homo sapiens
SYAMN

SEQ ID NO: 44 - protein, 17 aa, Homo sapiens
TISYSGSLISYADSVKG

SEQ ID NO: 45 - protein, 8 aa, Homo sapiens
INNDPMDV

SEQ ID NO: 46 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggccatggacgaggccgactactactgccagacctgggacgggaacccgacgcacggcgagaccaacgtgttcggcggagggaccaagttaaccgtccta

SEQ ID NO: 47 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQTWDGNPTHGETNVFGGGTKLTVL

SEQ ID NO: 48 - protein, 11 aa, Homo sapiens
SGDNLGKKYVQ

SEQ ID NO: 49 - protein, 7 aa, Homo sapiens
DDSNRPS

SEQ ID NO: 50 - protein, 14 aa, Homo sapiens
QTWDGNPTHGETNV

SEQ ID NO: 51 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 52 - protein, 117 aa, Homo sapiens
EVQLLESGGGLVQPGGSLRLSCAASGFTFSSYAMNWVRQAPGKGLEWVSTISYSGSLISYADSVKGRFTISRDNSKNTLYLQMNSLRAEDTAVYYCARIHHDPMDVWGQGTLVTVSS

SEQ ID NO: 53 - protein, 5 aa, Homo sapiens
SYAMN

SEQ ID NO: 54 - protein, 17 aa, Homo sapiens
TISYSGSLISYADSVKG

SEQ ID NO: 55 - protein, 8 aa, Homo sapiens
IHHDPMDV

SEQ ID NO: 56 - DNA, 333 bp, Homo sapiens; identical to SEQ ID NO: 16
  source 1..333: mol_type = other DNA

SEQ ID NO: 57 - protein, 111 aa, Homo sapiens; identical to SEQ ID NO: 7

SEQ ID NO: 58 - protein, 11 aa, Homo sapiens
SGDNLGKKYVQ

SEQ ID NO: 59 - protein, 7 aa, Homo sapiens
DDSNRPS

SEQ ID NO: 60 - protein, 14 aa, Homo sapiens
QTWDGNPTTGETNV

SEQ ID NO: 61 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatccaccacgaccctatggatcactggggccagggcaccctggtgacagtgagctca

SEQ ID NO: 62 - protein, 117 aa, Homo sapiens
EVQLLESGGGLVQPGGSLRLSCAASGFTFSSYAMNWVRQAPGKGLEWVSTISYSGSLISYADSVKGRFTISRDNSKNTLYLQMNSLRAEDTAVYYCARIHHDPMDHWGQGTLVTVSS

SEQ ID NO: 63 - protein, 5 aa, Homo sapiens
SYAMN

SEQ ID NO: 64 - protein, 17 aa, Homo sapiens
TISYSGSLISYADSVKG

SEQ ID NO: 65 - protein, 8 aa, Homo sapiens
IHHDPMDH

SEQ ID NO: 66 - DNA, 333 bp, Homo sapiens; identical to SEQ ID NO: 16
  source 1..333: mol_type = other DNA

SEQ ID NO: 67 - protein, 111 aa, Homo sapiens; identical to SEQ ID NO: 7

SEQ ID NO: 68 - protein, 11 aa, Homo sapiens
SGDNLGKKYVQ

SEQ ID NO: 69 - protein, 7 aa, Homo sapiens
DDSNRPS

SEQ ID NO: 70 - protein, 14 aa, Homo sapiens
QTWDGNPTTGETNV

SEQ ID NO: 71 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 72 - protein, 117 aa, Homo sapiens; identical to SEQ ID NO: 2

SEQ ID NO: 73 - protein, 5 aa, Homo sapiens
SYAMN

SEQ ID NO: 74 - protein, 17 aa, Homo sapiens
TISYSGSLISYADSVKG

SEQ ID NO: 75 - protein, 8 aa, Homo sapiens
INNDPMDV

SEQ ID NO: 76 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctggcaccacaacccgcacacgggggagaccaaccacttcggcggagggaccaagttaaccgtccta

SEQ ID NO: 77 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHTWHHNPHTGETNHFGGGTKLTVL

SEQ ID NO: 78 - protein, 11 aa, Homo sapiens
SGDNLGKKYVQ

SEQ ID NO: 79 - protein, 7 aa, Homo sapiens
DDSNRPS

SEQ ID NO: 80 - protein, 14 aa, Homo sapiens
HTWHHNPHTGETNH

SEQ ID NO: 81 - DNA, 351 bp, Homo sapiens; identical to SEQ ID NO: 71
  source 1..351: mol_type = other DNA

SEQ ID NO: 82 - protein, 117 aa, Homo sapiens; identical to SEQ ID NO: 2

SEQ ID NO: 83 - protein, 5 aa, Homo sapiens
SYAMN

SEQ ID NO: 84 - protein, 17 aa, Homo sapiens
TISYSGSLISYADSVKG

SEQ ID NO: 85 - protein, 8 aa, Homo sapiens
INNDPMDV

SEQ ID NO: 86 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccaccactggcacgggaaccaccacacgggggagaccaaccacttcggcggagggaccaagttaaccgtccta

SEQ ID NO: 87 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHHWHGNHHTGETNHFGGGTKLTVL

SEQ ID NO: 88 - protein, 11 aa, Homo sapiens
SGDNLGKKYVQ

SEQ ID NO: 89 - protein, 7 aa, Homo sapiens
DDSNRPS

SEQ ID NO: 90 - protein, 14 aa, Homo sapiens
HHWHGNHHTGETNH

SEQ ID NO: 91 - DNA, 351 bp, Homo sapiens; identical to SEQ ID NO: 71
  source 1..351: mol_type = other DNA

SEQ ID NO: 92 - protein, 117 aa, Homo sapiens; identical to SEQ ID NO: 2

SEQ ID NO: 93 - protein, 5 aa, Homo sapiens
SYAMN

SEQ ID NO: 94 - protein, 17 aa, Homo sapiens
TISYSGSLISYADSVKG

SEQ ID NO: 95 - protein, 8 aa, Homo sapiens
INNDPMDV

SEQ ID NO: 96 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctggcaccaccacccgacgcatggggagcacaacgtgttcggcggagggaccaagttaaccgtccta

SEQ ID NO: 97 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHTWHHHPTHGEHNVFGGGTKLTVL

SEQ ID NO: 98 - protein, 11 aa, Homo sapiens
SGDNLGKKYVQ

SEQ ID NO: 99 - protein, 7 aa, Homo sapiens
DDSNRPS

SEQ ID NO: 100 - protein, 14 aa, Homo sapiens
HTWHHHPTHGEHNV

SEQ ID NO: 101 - DNA, 351 bp, Homo sapiens; identical to SEQ ID NO: 71
  source 1..351: mol_type = other DNA

SEQ ID NO: 102 - protein, 117 aa, Homo sapiens; identical to SEQ ID NO: 2

SEQ ID NO: 103 - protein, 5 aa, Homo sapiens
SYAMN

SEQ ID NO: 104 - protein, 17 aa, Homo sapiens
TISYSGSLISYADSVKG

SEQ ID NO: 105 - protein, 8 aa, Homo sapiens
INNDPMDV

SEQ ID NO: 106 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagacctgggacgggcaccacacgcatggggagcaccacgtgttcggcggagggaccaagttaaccgtccta

SEQ ID NO: 107 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQTWDGHHTHGEHHVFGGGTKLTVL

SEQ ID NO: 108 - protein, 11 aa, Homo sapiens
SGDNLGKKYVQ

SEQ ID NO: 109 - protein, 7 aa, Homo sapiens
DDSNRPS

SEQ ID NO: 110 - protein, 14 aa, Homo sapiens
QTWDGHHTHGEHHV

SEQ ID NO: 111 - DNA, 351 bp, Homo sapiens; identical to SEQ ID NO: 71
  source 1..351: mol_type = other DNA

SEQ ID NO: 112 - protein, 117 aa, Homo sapiens; identical to SEQ ID NO: 2

SEQ ID NO: 113 - protein, 5 aa, Homo sapiens
SYAMN

SEQ ID NO: 114 - protein, 17 aa, Homo sapiens
TISYSGSLISYADSVKG

SEQ ID NO: 115 - protein, 8 aa, Homo sapiens
INNDPMDV

SEQ ID NO: 116 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagacctgggaccacaaccaccacacggggcacaccaacgtgttcggcggagggaccaagttaaccgtccta

SEQ ID NO: 117 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQTWDHNHHTGHTNVFGGGTKLTVL

SEQ ID NO: 118 - protein, 11 aa, Homo sapiens
SGDNLGKKYVQ

SEQ ID NO: 119 - protein, 7 aa, Homo sapiens
DDSNRPS

SEQ ID NO: 120 - protein, 14 aa, Homo sapiens
QTWDHNHHTGHTNV

SEQ ID NO: 121 - DNA, 351 bp, Homo sapiens; identical to SEQ ID NO: 71
  source 1..351: mol_type = other DNA

SEQ ID NO: 122 - protein, 117 aa, Homo sapiens; identical to SEQ ID NO: 2

SEQ ID NO: 123 - protein, 5 aa, Homo sapiens
SYAMN

SEQ ID NO: 124 - protein, 17 aa, Homo sapiens
TISYSGSLISYADSVKG

SEQ ID NO: 125 - protein, 8 aa, Homo sapiens
INNDPMDV

SEQ ID NO: 126 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctggcaccaccaccaccacacgggggagcacaaccacttcggcggagggaccaagttaaccgtccta

SEQ ID NO: 127 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHTWHHHHHTGEHNHFGGGTKLTVL

SEQ ID NO: 128 - protein, 11 aa, Homo sapiens
SGDNLGKKYVQ

SEQ ID NO: 129 - protein, 7 aa, Homo sapiens
DDSNRPS

SEQ ID NO: 130 - protein, 14 aa, Homo sapiens
HTWHHHHHTGEHNH

SEQ ID NO: 131 - DNA, 351 bp, Homo sapiens; identical to SEQ ID NO: 71
  source 1..351: mol_type = other DNA

SEQ ID NO: 132 - protein, 117 aa, Homo sapiens; identical to SEQ ID NO: 2

SEQ ID NO: 133 - protein, 5 aa, Homo sapiens
SYAMN

SEQ ID NO: 134 - protein, 17 aa, Homo sapiens
TISYSGSLISYADSVKG

SEQ ID NO: 135 - protein, 8 aa, Homo sapiens
INNDPMDV

SEQ ID NO: 136 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagcactggcacgggcacccgcaccatggggagcacaacgtgttcggcggagggaccaagttaaccgtccta

SEQ ID NO: 137 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQHWHGHPHHGEHNVFGGGTKLTVL

SEQ ID NO: 138 - protein, 11 aa, Homo sapiens
SGDNLGKKYVQ

SEQ ID NO: 139 - protein, 7 aa, Homo sapiens
DDSNRPS

SEQ ID NO: 140 - protein, 14 aa, Homo sapiens
QHWHGHPHHGEHNV

SEQ ID NO: 141 - DNA, 351 bp, Homo sapiens; identical to SEQ ID NO: 71
  source 1..351: mol_type = other DNA

SEQ ID NO: 142 - protein, 117 aa, Homo sapiens; identical to SEQ ID NO: 2

SEQ ID NO: 143 - protein, 5 aa, Homo sapiens
SYAMN

SEQ ID NO: 144 - protein, 17 aa, Homo sapiens
TISYSGSLISYADSVKG

SEQ ID NO: 145 - protein, 8 aa, Homo sapiens
INNDPMDV

SEQ ID NO: 146 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagcactgggacgggaaccaccaccatggggagaccaaccacttcggcggagggaccaagttaaccgtccta

SEQ ID NO: 147 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQHWDGNHHHGETNHFGGGTKLTVL

SEQ ID NO: 148 - protein, 11 aa, Homo sapiens
SGDNLGKKYVQ

SEQ ID NO: 149 - protein, 7 aa, Homo sapiens
DDSNRPS

SEQ ID NO: 150 - protein, 14 aa, Homo sapiens
QHWDGNHHHGETNH

SEQ ID NO: 151 - DNA, 351 bp, Homo sapiens; identical to SEQ ID NO: 71
  source 1..351: mol_type = other DNA

SEQ ID NO: 152 - protein, 117 aa, Homo sapiens; identical to SEQ ID NO: 2

SEQ ID NO: 153 - protein, 5 aa, Homo sapiens
SYAMN

SEQ ID NO: 154 - protein, 17 aa, Homo sapiens
TISYSGSLISYADSVKG

SEQ ID NO: 155 - protein, 8 aa, Homo sapiens
INNDPMDV

SEQ ID NO: 156 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctgggaccaccaccacacgacgggggagcacaacgtgttcggcggagggaccaagttaaccgtccta

SEQ ID NO: 157 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHTWDHHHTTGEHNVFGGGTKLTVL

SEQ ID NO: 158 - protein, 11 aa, Homo sapiens
SGDNLGKKYVQ

SEQ ID NO: 159 - protein, 7 aa, Homo sapiens
DDSNRPS

SEQ ID NO: 160 - protein, 14 aa, Homo sapiens
HTWDHHHTTGEHNV

SEQ ID NO: 161 - DNA, 351 bp, Homo sapiens; identical to SEQ ID NO: 71
  source 1..351: mol_type = other DNA

SEQ ID NO: 162 - protein, 117 aa, Homo sapiens; identical to SEQ ID NO: 2

SEQ ID NO: 163 - protein, 5 aa, Homo sapiens
SYAMN

SEQ ID NO: 164 - protein, 17 aa, Homo sapiens
TISYSGSLISYADSVKG

SEQ ID NO: 165 - protein, 8 aa, Homo sapiens
INNDPMDV

SEQ ID NO: 166 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccaccactggcacgggcaccacacgacgggggagaccaaccacttcggcggagggaccaagttaaccgtccta

SEQ ID NO: 167 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHHWHGHHTTGETNHFGGGTKLTVL

SEQ ID NO: 168 - protein, 11 aa, Homo sapiens
SGDNLGKKYVQ

SEQ ID NO: 169 - protein, 7 aa, Homo sapiens
DDSNRPS

SEQ ID NO: 170 - protein, 14 aa, Homo sapiens
HHWHGHHTTGETNH

SEQ ID NO: 171 - DNA, 351 bp, Homo sapiens; identical to SEQ ID NO: 71
  source 1..351: mol_type = other DNA

SEQ ID NO: 172 - protein, 117 aa, Homo sapiens; identical to SEQ ID NO: 2

SEQ ID NO: 173 - protein, 5 aa, Homo sapiens
SYAMN

SEQ ID NO: 174 - protein, 17 aa, Homo sapiens
TISYSGSLISYADSVKG

SEQ ID NO: 175 - protein, 8 aa, Homo sapiens
INNDPMDV

SEQ ID NO: 176 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccaccactggcacgggcacccgcacacgggggagcacaacgtgttcggcggagggaccaagttaaccgtccta

SEQ ID NO: 177 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHHWHGHPHTGEHNVFGGGTKLTVL

SEQ ID NO: 178 - protein, 11 aa, Homo sapiens
SGDNLGKKYVQ

SEQ ID NO: 179 - protein, 7 aa, Homo sapiens
DDSNRPS

SEQ ID NO: 180 - protein, 14 aa, Homo sapiens
HHWHGHPHTGEHNV

SEQ ID NO: 181 - DNA, 351 bp, Homo sapiens; identical to SEQ ID NO: 71
  source 1..351: mol_type = other DNA

SEQ ID NO: 182 - protein, 117 aa, Homo sapiens; identical to SEQ ID NO: 2

SEQ ID NO: 183 - protein, 5 aa, Homo sapiens
SYAMN

SEQ ID NO: 184 - protein, 17 aa, Homo sapiens
TISYSGSLISYADSVKG

SEQ ID NO: 185 - protein, 8 aa, Homo sapiens
INNDPMDV

SEQ ID NO: 186 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagcactgggacgggcaccacacgacgggggagaccaaccacttcggcggagggaccaagttaaccgtccta

SEQ ID NO: 187 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQHWDGHHTTGETNHFGGGTKLTVL

SEQ ID NO: 188 - protein, 11 aa, Homo sapiens
SGDNLGKKYVQ

SEQ ID NO: 189 - protein, 7 aa, Homo sapiens
DDSNRPS

SEQ ID NO: 190 - protein, 14 aa, Homo sapiens
QHWDGHHTTGETNH

SEQ ID NO: 191 - DNA, 351 bp, Homo sapiens; identical to SEQ ID NO: 71
  source 1..351: mol_type = other DNA

SEQ ID NO: 192 - protein, 117 aa, Homo sapiens; identical to SEQ ID NO: 2

SEQ ID NO: 193 - protein, 5 aa, Homo sapiens
SYAMN

SEQ ID NO: 194 - protein, 17 aa, Homo sapiens
TISYSGSLISYADSVKG

SEQ ID NO: 195 - protein, 8 aa, Homo sapiens
INNDPMDV

SEQ ID NO: 196 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagacctgggacgggcaccaccacacgggggagaccaaccacttcggcggagggaccaagttaaccgtccta

SEQ ID NO: 197 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQTWDGHHHTGETNHFGGGTKLTVL

SEQ ID NO: 198 - protein, 11 aa, Homo sapiens
SGDNLGKKYVQ

SEQ ID NO: 199 - protein, 7 aa, Homo sapiens
DDSNRPS

SEQ ID NO: 200 - protein, 14 aa, Homo sapiens
QTWDGHHHTGETNH

SEQ ID NO: 201 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcaccacatcagccaccacgacagccatcatggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 202 - protein, 117 aa, Homo sapiens
EVQLLESGGGLVQPGGSLRLSCAASGFTFSSYAMNWVRQAPGKGLEWVSTISHSGHHISHHDSHHGRFTISRDNSKNTLYLQMNSLRAEDTAVYYCARINNDPMDVWGQGTLVTVSS

SEQ ID NO: 203 - protein, 5 aa, Homo sapiens
SYAMN

SEQ ID NO: 204 - protein, 17 aa, Homo sapiens
TISHSGHHISHHDSHHG

SEQ ID NO: 205 - protein, 8 aa, Homo sapiens
INNDPMDV

SEQ ID NO: 206 - DNA, 333 bp, Homo sapiens; identical to SEQ ID NO: 16
  source 1..333: mol_type = other DNA

SEQ ID NO: 207 - protein, 111 aa, Homo sapiens; identical to SEQ ID NO: 7

SEQ ID NO: 208 - protein, 11 aa, Homo sapiens
SGDNLGKKYVQ

SEQ ID NO: 209 - protein, 7 aa, Homo sapiens
DDSNRPS

SEQ ID NO: 210 - protein, 14 aa, Homo sapiens
QTWDGNPTTGETNV

SEQ ID NO: 211 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagccactcgctgatcagctacgcccacagcgtgcatggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 212 - protein, 117 aa, Homo sapiens
EVQLLESGGGLVQPGGSLRLSCAASGFTFSSYAMNWVRQAPGKGLEWVSTISHSHSLISYAHSVHGRFTISRDNSKNTLYLQMNSLRAEDTAVYYCARINNDPMDVWGQGTLVTVSS

SEQ ID NO: 213 - protein, 5 aa, Homo sapiens
SYAMN

SEQ ID NO: 214 - protein, 17 aa, Homo sapiens
TISHSHSLISYAHSVHG

SEQ ID NO: 215 - protein, 8 aa, Homo sapiens
INNDPMDV

SEQ ID NO: 216 - DNA, 333 bp, Homo sapiens; identical to SEQ ID NO: 16
  source 1..333: mol_type = other DNA

SEQ ID NO: 217 - protein, 111 aa, Homo sapiens; identical to SEQ ID NO: 7

SEQ ID NO: 218 - protein, 11 aa, Homo sapiens
SGDNLGKKYVQ

SEQ ID NO: 219 - protein, 7 aa, Homo sapiens
DDSNRPS

SEQ ID NO: 220 - protein, 14 aa, Homo sapiens
QTWDGNPTTGETNV

SEQ ID NO: 221 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatccaccacagcgggcacctgcacagccacgccgaccacgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 222 - protein, 117 aa, Homo sapiens
EVQLLESGGGLVQPGGSLRLSCAASGFTFSSYAMNWVRQAPGKGLEWVSTIHHSGHLHSHADHVKGRFTISRDNSKNTLYLQMNSLRAEDTAVYYCARINNDPMDVWGQGTLVTVSS

SEQ ID NO: 223 - protein, 5 aa, Homo sapiens
SYAMN

SEQ ID NO: 224 - protein, 17 aa, Homo sapiens
TIHHSGHLHSHADHVKG

SEQ ID NO: 225 - protein, 8 aa, Homo sapiens
INNDPMDV

SEQ ID NO: 226 - DNA, 333 bp, Homo sapiens; identical to SEQ ID NO: 16
  source 1..333: mol_type = other DNA

SEQ ID NO: 227 - protein, 111 aa, Homo sapiens; identical to SEQ ID NO: 7

SEQ ID NO: 228 - protein, 11 aa, Homo sapiens
SGDNLGKKYVQ

SEQ ID NO: 229 - protein, 7 aa, Homo sapiens
DDSNRPS

SEQ ID NO: 230 - protein, 14 aa, Homo sapiens
QTWDGNPTTGETNV

SEQ ID NO: 231 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcacctgatccaccacgccgacagccataagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 232 - protein, 117 aa, Homo sapiens
EVQLLESGGGLVQPGGSLRLSCAASGFTFSSYAMNWVRQAPGKGLEWVSTISHSGHLIHHADSHKGRFTISRDNSKNTLYLQMNSLRAEDTAVYYCARINNDPMDVWGQGTLVTVSS

SEQ ID NO: 233 - protein, 5 aa, Homo sapiens
SYAMN

SEQ ID NO: 234 - protein, 17 aa, Homo sapiens
TISHSGHLIHHADSHKG

SEQ ID NO: 235 - protein, 8 aa, Homo sapiens
INNDPMDV

SEQ ID NO: 236 - DNA, 333 bp, Homo sapiens; identical to SEQ ID NO: 16
  source 1..333: mol_type = other DNA

SEQ ID NO: 237 - protein, 111 aa, Homo sapiens; identical to SEQ ID NO: 7

SEQ ID NO: 238 - protein, 11 aa, Homo sapiens
SGDNLGKKYVQ

SEQ ID NO: 239 - protein, 7 aa, Homo sapiens
DDSNRPS

SEQ ID NO: 240 - protein, 14 aa, Homo sapiens
QTWDGNPTTGETNV

SEQ ID NO: 241 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccaccacagccataagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 242 - protein, 117 aa, Homo sapiens
EVQLLESGGGLVQPGGSLRLSCAASGFTFSSYAMNWVRQAPGKGLEWVSTISYHGSHISYHHSHKGRFTISRDNSKNTLYLQMNSLRAEDTAVYYCARINNDPMDVWGQGTLVTVSS

SEQ ID NO: 243 - protein, 5 aa, Homo sapiens
SYAMN

SEQ ID NO: 244 - protein, 17 aa, Homo sapiens
TISYHGSHISYHHSHKG

SEQ ID NO: 245 - protein, 8 aa, Homo sapiens
INNDPMDV

SEQ ID NO: 246 - DNA, 333 bp, Homo sapiens; identical to SEQ ID NO: 16
  source 1..333: mol_type = other DNA

SEQ ID NO: 247 - protein, 111 aa, Homo sapiens; identical to SEQ ID NO: 7

SEQ ID NO: 248 - protein, 11 aa, Homo sapiens
SGDNLGKKYVQ

SEQ ID NO: 249 - protein, 7 aa, Homo sapiens
DDSNRPS

SEQ ID NO: 250 - protein, 14 aa, Homo sapiens
QTWDGNPTTGETNV

SEQ ID NO: 251 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctacgccgaccaccataagcacaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 252 - protein, 117 aa, Homo sapiens
EVQLLESGGGLVQPGGSLRLSCAASGFTFSSYAMNWVRQAPGKGLEWVSTISYHGSHISYADHHKHRFTISRDNSKNTLYLQMNSLRAEDTAVYYCARINNDPMDVWGQGTLVTVSS

SEQ ID NO: 253 - protein, 5 aa, Homo sapiens
SYAMN

SEQ ID NO: 254 - protein, 17 aa, Homo sapiens
TISYHGSHISYADHHKH

SEQ ID NO: 255 - protein, 8 aa, Homo sapiens
INNDPMDV

SEQ ID NO: 256 - DNA, 333 bp, Homo sapiens; identical to SEQ ID NO: 16
  source 1..333: mol_type = other DNA

SEQ ID NO: 257 - protein, 111 aa, Homo sapiens; identical to SEQ ID NO: 7

SEQ ID NO: 258 - protein, 11 aa, Homo sapiens
SGDNLGKKYVQ

SEQ ID NO: 259 - protein, 7 aa, Homo sapiens
DDSNRPS

SEQ ID NO: 260 - protein, 14 aa, Homo sapiens
QTWDGNPTTGETNV

SEQ ID NO: 261 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggcacctgatccactacgccgaccaccatcatggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 262 - protein, 117 aa, Homo sapiens
EVQLLESGGGLVQPGGSLRLSCAASGFTFSSYAMNWVRQAPGKGLEWVSTISYSGHLIHYADHHHGRFTISRDNSKNTLYLQMNSLRAEDTAVYYCARINNDPMDVWGQGTLVTVSS

SEQ ID NO: 263 - protein, 5 aa, Homo sapiens
SYAMN

SEQ ID NO: 264 - protein, 17 aa, Homo sapiens
TISYSGHLIHYADHHHG

SEQ ID NO: 265 - protein, 8 aa, Homo sapiens
INNDPMDV

SEQ ID NO: 266 - DNA, 333 bp, Homo sapiens; identical to SEQ ID NO: 16
  source 1..333: mol_type = other DNA

SEQ ID NO: 267 - protein, 111 aa, Homo sapiens; identical to SEQ ID NO: 7

SEQ ID NO: 268 - protein, 11 aa, Homo sapiens
SGDNLGKKYVQ

SEQ ID NO: 269 - protein, 7 aa, Homo sapiens
DDSNRPS

SEQ ID NO: 270 - protein, 14 aa, Homo sapiens
QTWDGNPTTGETNV

SEQ ID NO: 271 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagccacgccgaccacgtgcatggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 272 - protein, 117 aa, Homo sapiens
EVQLLESGGGLVQPGGSLRLSCAASGFTFSSYAMNWVRQAPGKGLEWVSTISYHGSHISHADHVHGRFTISRDNSKNTLYLQMNSLRAEDTAVYYCARINNDPMDVWGQGTLVTVSS

SEQ ID NO: 273 - protein, 5 aa, Homo sapiens
SYAMN

SEQ ID NO: 274 - protein, 17 aa, Homo sapiens
TISYHGSHISHADHVHG

SEQ ID NO: 275 - protein, 8 aa, Homo sapiens
INNDPMDV

SEQ ID NO: 276 - DNA, 333 bp, Homo sapiens; identical to SEQ ID NO: 16
  source 1..333: mol_type = other DNA

SEQ ID NO: 277 - protein, 111 aa, Homo sapiens; identical to SEQ ID NO: 7

SEQ ID NO: 278 - protein, 11 aa, Homo sapiens
SGDNLGKKYVQ

SEQ ID NO: 279 - protein, 7 aa, Homo sapiens
DDSNRPS

SEQ ID NO: 280 - protein, 14 aa, Homo sapiens
QTWDGNPTTGETNV

SEQ ID NO: 281 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagccacgccgaccacgtgaagcacaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 282 - protein, 117 aa, Homo sapiens
EVQLLESGGGLVQPGGSLRLSCAASGFTFSSYAMNWVRQAPGKGLEWVSTISYHGHHISHADHVKHRFTISRDNSKNTLYLQMNSLRAEDTAVYYCARINNDPMDVWGQGTLVTVSS

SEQ ID NO: 283 - protein, 5 aa, Homo sapiens
SYAMN

SEQ ID NO: 284 - protein, 17 aa, Homo sapiens
TISYHGHHISHADHVKH

SEQ ID NO: 285 - protein, 8 aa, Homo sapiens
INNDPMDV

SEQ ID NO: 286 - DNA, 333 bp, Homo sapiens; identical to SEQ ID NO: 16
  source 1..333: mol_type = other DNA

SEQ ID NO: 287 - protein, 111 aa, Homo sapiens; identical to SEQ ID NO: 7

SEQ ID NO: 288 - protein, 11 aa, Homo sapiens
SGDNLGKKYVQ

SEQ ID NO: 289 - protein, 7 aa, Homo sapiens
DDSNRPS

SEQ ID NO: 290 - protein, 14 aa, Homo sapiens
QTWDGNPTTGETNV

SEQ ID NO: 291 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcacctgatccaccaccaccacagcgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 292 - protein, 117 aa, Homo sapiens
EVQLLESGGGLVQPGGSLRLSCAASGFTFSSYAMNWVRQAPGKGLEWVSTISHSGHLIHHHHSVKGRFTISRDNSKNTLYLQMNSLRAEDTAVYYCARINNDPMDVWGQGTLVTVSS

SEQ ID NO: 293 - protein, 5 aa, Homo sapiens
SYAMN

SEQ ID NO: 294 - protein, 17 aa, Homo sapiens
TISHSGHLIHHHHSVKG

SEQ ID NO: 295 - protein, 8 aa, Homo sapiens
INNDPMDV

SEQ ID NO: 296 - DNA, 333 bp, Homo sapiens; identical to SEQ ID NO: 16
  source 1..333: mol_type = other DNA

SEQ ID NO: 297 - protein, 111 aa, Homo sapiens; identical to SEQ ID NO: 7

SEQ ID NO: 298 - protein, 11 aa, Homo sapiens
SGDNLGKKYVQ

SEQ ID NO: 299 - protein, 7 aa, Homo sapiens
DDSNRPS

SEQ ID NO: 300 - protein, 14 aa, Homo sapiens
QTWDGNPTTGETNV

SEQ ID NO: 301 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagccatcatggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 302 - protein, 117 aa, Homo sapiens
EVQLLESGGGLVQPGGSLRLSCAASGFTFSSYAMNWVRQAPGKGLEWVSTISYHGSHISYHDSHHGRFTISRDNSKNTLYLQMNSLRAEDTAVYYCARINNDPMDVWGQGTLVTVSS

SEQ ID NO: 303 - protein, 5 aa, Homo sapiens
SYAMN

SEQ ID NO: 304 - protein, 17 aa, Homo sapiens
TISYHGSHISYHDSHHG

SEQ ID NO: 305 - protein, 8 aa, Homo sapiens
INNDPMDV

SEQ ID NO: 306 - DNA, 333 bp, Homo sapiens; identical to SEQ ID NO: 16
  source 1..333: mol_type = other DNA

SEQ ID NO: 307 - protein, 111 aa, Homo sapiens; identical to SEQ ID NO: 7

SEQ ID NO: 308 - protein, 11 aa, Homo sapiens
SGDNLGKKYVQ

SEQ ID NO: 309 - protein, 7 aa, Homo sapiens
DDSNRPS

SEQ ID NO: 310 - protein, 14 aa, Homo sapiens
QTWDGNPTTGETNV

SEQ ID NO: 311 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccaccaccacctgatcagccacgccgacagcgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 312 - protein, 117 aa, Homo sapiens
EVQLLESGGGLVQPGGSLRLSCAASGFTFSSYAMNWVRQAPGKGLEWVSTISYHHHLISHADSVKGRFTISRDNSKNTLYLQMNSLRAEDTAVYYCARINNDPMDVWGQGTLVTVSS

SEQ ID NO: 313 - protein, 5 aa, Homo sapiens
SYAMN

SEQ ID NO: 314 - protein, 17 aa, Homo sapiens
TISYHHHLISHADSVKG

SEQ ID NO: 315 - protein, 8 aa, Homo sapiens
INNDPMDV

SEQ ID NO: 316 - DNA, 333 bp, Homo sapiens; identical to SEQ ID NO: 16
  source 1..333: mol_type = other DNA

SEQ ID NO: 317 - protein, 111 aa, Homo sapiens; identical to SEQ ID NO: 7

SEQ ID NO: 318 - protein, 11 aa, Homo sapiens
SGDNLGKKYVQ

SEQ ID NO: 319 - protein, 7 aa, Homo sapiens
DDSNRPS

SEQ ID NO: 320 - protein, 14 aa, Homo sapiens
QTWDGNPTTGETNV

SEQ ID NO: 321 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagccacgccgaccaccataagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 322 - protein, 117 aa, Homo sapiens
EVQLLESGGGLVQPGGSLRLSCAASGFTFSSYAMNWVRQAPGKGLEWVSTISYHGHHISHADHHKGRFTISRDNSKNTLYLQMNSLRAEDTAVYYCARINNDPMDVWGQGTLVTVSS

SEQ ID NO: 323 - protein, 5 aa, Homo sapiens
SYAMN

SEQ ID NO: 324 - protein, 17 aa, Homo sapiens
TISYHGHHISHADHHKG

SEQ ID NO: 325 - protein, 8 aa, Homo sapiens
INNDPMDV

SEQ ID NO: 326 - DNA, 333 bp, Homo sapiens; identical to SEQ ID NO: 16
  source 1..333: mol_type = other DNA

SEQ ID NO: 327 - protein, 111 aa, Homo sapiens; identical to SEQ ID NO: 7

SEQ ID NO: 328 - protein, 11 aa, Homo sapiens
SGDNLGKKYVQ

SEQ ID NO: 329 - protein, 7 aa, Homo sapiens
DDSNRPS

SEQ ID NO: 330 - protein, 14 aa, Homo sapiens
QTWDGNPTTGETNV

SEQ ID NO: 331 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagctacgccgacagccatcatcacaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 332 - protein, 117 aa, Homo sapiens
EVQLLESGGGLVQPGGSLRLSCAASGFTFSSYAMNWVRQAPGKGLEWVSTISYHGHHISYADSHHHRFTISRDNSKNTLYLQMNSLRAEDTAVYYCARINNDPMDVWGQGTLVTVSS

SEQ ID NO: 333 - protein, 5 aa, Homo sapiens
SYAMN

SEQ ID NO: 334 - protein, 17 aa, Homo sapiens
TISYHGHHISYADSHHH

SEQ ID NO: 335 - protein, 8 aa, Homo sapiens
INNDPMDV

SEQ ID NO: 336 - DNA, 333 bp, Homo sapiens; identical to SEQ ID NO: 16
  source 1..333: mol_type = other DNA

SEQ ID NO: 337 - protein, 111 aa, Homo sapiens; identical to SEQ ID NO: 7

SEQ ID NO: 338 - protein, 11 aa, Homo sapiens
SGDNLGKKYVQ

SEQ ID NO: 339 - protein, 7 aa, Homo sapiens
DDSNRPS

SEQ ID NO: 340 - protein, 14 aa, Homo sapiens
QTWDGNPTTGETNV

SEQ ID NO: 341 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatccaccacagcgggcacctgcacagctaccaccaccacgtgaagggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 342 - protein, 117 aa, Homo sapiens
EVQLLESGGGLVQPGGSLRLSCAASGFTFSSYAMNWVRQAPGKGLEWVSTIHHSGHLHSYHHHVKGRFTISRDNSKNTLYLQMNSLRAEDTAVYYCARINNDPMDVWGQGTLVTVSS

SEQ ID NO: 343 - protein, 5 aa, Homo sapiens
SYAMN

SEQ ID NO: 344 - protein, 17 aa, Homo sapiens
TIHHSGHLHSYHHHVKG

SEQ ID NO: 345 - protein, 8 aa, Homo sapiens
INNDPMDV

SEQ ID NO: 346 - DNA, 333 bp, Homo sapiens; identical to SEQ ID NO: 16
  source 1..333: mol_type = other DNA

SEQ ID NO: 347 - protein, 111 aa, Homo sapiens; identical to SEQ ID NO: 7

SEQ ID NO: 348 - protein, 11 aa, Homo sapiens
SGDNLGKKYVQ

SEQ ID NO: 349 - protein, 7 aa, Homo sapiens
DDSNRPS

SEQ ID NO: 350 - protein, 14 aa, Homo sapiens
QTWDGNPTTGETNV

SEQ ID NO: 351 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcacctgcaccactacgccgacagccataagcacaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 352 - protein, 117 aa, Homo sapiens
EVQLLESGGGLVQPGGSLRLSCAASGFTFSSYAMNWVRQAPGKGLEWVSTISHSGHLHHYADSHKHRFTISRDNSKNTLYLQMNSLRAEDTAVYYCARINNDPMDVWGQGTLVTVSS

SEQ ID NO: 353 - protein, 5 aa, Homo sapiens
SYAMN

SEQ ID NO: 354 - protein, 17 aa, Homo sapiens
TISHSGHLHHYADSHKH

SEQ ID NO: 355 - protein, 8 aa, Homo sapiens
INNDPMDV

SEQ ID NO: 356 - DNA, 333 bp, Homo sapiens; identical to SEQ ID NO: 16
  source 1..333: mol_type = other DNA

SEQ ID NO: 357 - protein, 111 aa, Homo sapiens; identical to SEQ ID NO: 7

SEQ ID NO: 358 - protein, 11 aa, Homo sapiens
SGDNLGKKYVQ

SEQ ID NO: 359 - protein, 7 aa, Homo sapiens
DDSNRPS

SEQ ID NO: 360 - protein, 14 aa, Homo sapiens
QTWDGNPTTGETNV

SEQ ID NO: 361 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgcatcacaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 362 - protein, 117 aa, Homo sapiens
EVQLLESGGGLVQPGGSLRLSCAASGFTFSSYAMNWVRQAPGKGLEWVSTISYHGSHISYHDSVHHRFTISRDNSKNTLYLQMNSLRAEDTAVYYCARINNDPMDVWGQGTLVTVSS

SEQ ID NO: 363 - protein, 5 aa, Homo sapiens
SYAMN

SEQ ID NO: 364 - protein, 17 aa, Homo sapiens
TISYHGSHISYHDSVHH

SEQ ID NO: 365 - protein, 8 aa, Homo sapiens
INNDPMDV

SEQ ID NO: 366 - DNA, 333 bp, Homo sapiens; identical to SEQ ID NO: 16
  source 1..333: mol_type = other DNA

SEQ ID NO: 367 - protein, 111 aa, Homo sapiens; identical to SEQ ID NO: 7

SEQ ID NO: 368 - protein, 11 aa, Homo sapiens
SGDNLGKKYVQ

SEQ ID NO: 369 - protein, 7 aa, Homo sapiens
DDSNRPS

SEQ ID NO: 370 - protein, 14 aa, Homo sapiens
QTWDGNPTTGETNV

SEQ ID NO: 371 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagccacgcccaccacgtgcatggcaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtgacagtgagctca

SEQ ID NO: 372 - protein, 117 aa, Homo sapiens
EVQLLESGGGLVQPGGSLRLSCAASGFTFSSYAMNWVRQAPGKGLEWVSTISYHGHHISHAHHVHGRFTISRDNSKNTLYLQMNSLRAEDTAVYYCARINNDPMDVWGQGTLVTVSS

SEQ ID NO: 373 - protein, 5 aa, Homo sapiens
SYAMN

SEQ ID NO: 374 - protein, 17 aa, Homo sapiens
TISYHGHHISHAHHVHG

SEQ ID NO: 375 - protein, 8 aa, Homo sapiens
INNDPMDV

SEQ ID NO: 376 - DNA, 333 bp, Homo sapiens; identical to SEQ ID NO: 16
  source 1..333: mol_type = other DNA

SEQ ID NO: 377 - protein, 111 aa, Homo sapiens; identical to SEQ ID NO: 7

SEQ ID NO: 378 - protein, 11 aa, Homo sapiens
SGDNLGKKYVQ

SEQ ID NO: 379 - protein, 7 aa, Homo sapiens
DDSNRPS

SEQ ID NO: 380 - protein, 14 aa, Homo sapiens
QTWDGNPTTGETNV

SEQ ID NO: 381 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatccaccaccaccctcacgatcactggggccagggcaccctggtgacagtgagctca

SEQ ID NO: 382 - protein, 117 aa, Homo sapiens
EVQLLESGGGLVQPGGSLRLSCAASGFTFSSYAMNWVRQAPGKGLEWVSTISYSGSLISYADSVKGRFTISRDNSKNTLYLQMNSLRAEDTAVYYCARIHHHPHDHWGQGTLVTVSS

SEQ ID NO: 383 - protein, 5 aa, Homo sapiens
SYAMN

SEQ ID NO: 384 - protein, 17 aa, Homo sapiens
TISYSGSLISYADSVKG

SEQ ID NO: 385 - protein, 8 aa, Homo sapiens
IHHHPHDH

SEQ ID NO: 386 - DNA, 333 bp, Homo sapiens; identical to SEQ ID NO: 16
  source 1..333: mol_type = other DNA

SEQ ID NO: 387 - protein, 111 aa, Homo sapiens; identical to SEQ ID NO: 7

SEQ ID NO: 388 - protein, 11 aa, Homo sapiens
SGDNLGKKYVQ

SEQ ID NO: 389 - protein, 7 aa, Homo sapiens
DDSNRPS

SEQ ID NO: 390 - protein, 14 aa, Homo sapiens
QTWDGNPTTGETNV

SEQ ID NO: 391 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatcaaccacgaccctatggatcactggggccagggcaccctggtgacagtgagctca

SEQ ID NO: 392 - protein, 117 aa, Homo sapiens
EVQLLESGGGLVQPGGSLRLSCAASGFTFSSYAMNWVRQAPGKGLEWVSTISYSGSLISYADSVKGRFTISRDNSKNTLYLQMNSLRAEDTAVYYCARINHDPMDHWGQGTLVTVSS

SEQ ID NO: 393 - protein, 5 aa, Homo sapiens
SYAMN

SEQ ID NO: 394 - protein, 17 aa, Homo sapiens
TISYSGSLISYADSVKG

SEQ ID NO: 395 - protein, 8 aa, Homo sapiens
INHDPMDH

SEQ ID NO: 396 - DNA, 333 bp, Homo sapiens; identical to SEQ ID NO: 16
  source 1..333: mol_type = other DNA

SEQ ID NO: 397 - protein, 111 aa, Homo sapiens; identical to SEQ ID NO: 7

SEQ ID NO: 398 - protein, 11 aa, Homo sapiens
SGDNLGKKYVQ

SEQ ID NO: 399 - protein, 7 aa, Homo sapiens
DDSNRPS

SEQ ID NO: 400 - protein, 14 aa, Homo sapiens
QTWDGNPTTGETNV

SEQ ID NO: 401 - DNA, 351 bp, Homo sapiens; identical to SEQ ID NO: 71
  source 1..351: mol_type = other DNA

SEQ ID NO: 402 - protein, 117 aa, Homo sapiens; identical to SEQ ID NO: 2

SEQ ID NO: 403 - protein, 5 aa, Homo sapiens
SYAMN

SEQ ID NO: 404 - protein, 17 aa, Homo sapiens
TISYSGSLISYADSVKG

SEQ ID NO: 405 - protein, 8 aa, Homo sapiens
INNDPMDV

SEQ ID NO: 406 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctgggacgggcaccacacgacgggggagaccaaccacttcggcggagggaccaagttaaccgtccta

SEQ ID NO: 407 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHTWDGHHTTGETNHFGGGTKLTVL

SEQ ID NO: 408 - protein, 11 aa, Homo sapiens
SGDNLGKKYVQ

SEQ ID NO: 409 - protein, 7 aa, Homo sapiens
DDSNRPS

SEQ ID NO: 410 - protein, 14 aa, Homo sapiens
HTWDGHHTTGETNH

SEQ ID NO: 411 - DNA, 351 bp, Homo sapiens; identical to SEQ ID NO: 71
  source 1..351: mol_type = other DNA

SEQ ID NO: 412 - protein, 117 aa, Homo sapiens; identical to SEQ ID NO: 2

SEQ ID NO: 413 - protein, 5 aa, Homo sapiens
SYAMN

SEQ ID NO: 414 - protein, 17 aa, Homo sapiens
TISYSGSLISYADSVKG

SEQ ID NO: 415 - protein, 8 aa, Homo sapiens
INNDPMDV

SEQ ID NO: 416 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagcactgggacgggcaccaccacacgggggagaccaacgtgttcggcggagggaccaagttaaccgtccta

SEQ ID NO: 417 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQHWDGHHHTGETNVFGGGTKLTVL

SEQ ID NO: 418 - protein, 11 aa, Homo sapiens
SGDNLGKKYVQ

SEQ ID NO: 419 - protein, 7 aa, Homo sapiens
DDSNRPS

SEQ ID NO: 420 - protein, 14 aa, Homo sapiens
QHWDGHHHTGETNV

SEQ ID NO: 421 - DNA, 351 bp, Homo sapiens; identical to SEQ ID NO: 71
  source 1..351: mol_type = other DNA

SEQ ID NO: 422 - protein, 117 aa, Homo sapiens; identical to SEQ ID NO: 2

SEQ ID NO: 423 - protein, 5 aa, Homo sapiens
SYAMN

SEQ ID NO: 424 - protein, 17 aa, Homo sapiens
TISYSGSLISYADSVKG

SEQ ID NO: 425 - protein, 8 aa, Homo sapiens
INNDPMDV

SEQ ID NO: 426 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagcactggcacgggaacccgcacacgggggagcacaaccacttcggcggagggaccaagttaaccgtccta

SEQ ID NO: 427 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQHWHGNPHTGEHNHFGGGTKLTVL

SEQ ID NO: 428 - protein, 11 aa, Homo sapiens
SGDNLGKKYVQ

SEQ ID NO: 429 - protein, 7 aa, Homo sapiens
DDSNRPS

SEQ ID NO: 430 - protein, 14 aa, Homo sapiens
QHWHGNPHTGEHNH

SEQ ID NO: 431 - DNA, 351 bp, Homo sapiens; identical to SEQ ID NO: 71
  source 1..351: mol_type = other DNA

SEQ ID NO: 432 - protein, 117 aa, Homo sapiens; identical to SEQ ID NO: 2

SEQ ID NO: 433 - protein, 5 aa, Homo sapiens
SYAMN

SEQ ID NO: 434 - protein, 17 aa, Homo sapiens
TISYSGSLISYADSVKG

SEQ ID NO: 435 - protein, 8 aa, Homo sapiens
INNDPMDV

SEQ ID NO: 436 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctgggaccacaacccgcaccatcatcacaccaaccacttcggcggagggaccaagttaaccgtccta

SEQ ID NO: 437 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHTWDHNPHHHHTNHFGGGTKLTVL

SEQ ID NO: 438 - protein, 11 aa, Homo sapiens
SGDNLGKKYVQ

SEQ ID NO: 439 - protein, 7 aa, Homo sapiens
DDSNRPS

SEQ ID NO: 440 - protein, 14 aa, Homo sapiens
HTWDHNPHHHHTNH

SEQ ID NO: 441 - DNA, 351 bp, Homo sapiens; identical to SEQ ID NO: 71
  source 1..351: mol_type = other DNA

SEQ ID NO: 442 - protein, 117 aa, Homo sapiens; identical to SEQ ID NO: 2

SEQ ID NO: 443 - protein, 5 aa, Homo sapiens
SYAMN

SEQ ID NO: 444 - protein, 17 aa, Homo sapiens
TISYSGSLISYADSVKG

SEQ ID NO: 445 - protein, 8 aa, Homo sapiens
INNDPMDV

SEQ ID NO: 446 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccaccactggcaccacaacccgacgcatcatgagaccaacgtgttcggcggagggaccaagttaaccgtccta

SEQ ID NO: 447 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCHHWHHNPTHHETNVFGGGTKLTVL

SEQ ID NO: 448 - protein, 11 aa, Homo sapiens
SGDNLGKKYVQ

SEQ ID NO: 449 - protein, 7 aa, Homo sapiens
DDSNRPS

SEQ ID NO: 450 - protein, 14 aa, Homo sapiens
HHWHHNPTHHETNV

SEQ ID NO: 451 - DNA, 351 bp, Homo sapiens; identical to SEQ ID NO: 71
  source 1..351: mol_type = other DNA

SEQ ID NO: 452 - protein, 117 aa, Homo sapiens; identical to SEQ ID NO: 2

SEQ ID NO: 453 - protein, 5 aa, Homo sapiens
SYAMN

SEQ ID NO: 454 - protein, 17 aa, Homo sapiens
TISYSGSLISYADSVKG

SEQ ID NO: 455 - protein, 8 aa, Homo sapiens
INNDPMDV

SEQ ID NO: 456 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagcactggcacgggcaccaccacacgggggagaccaacgtgttcggcggagggaccaagttaaccgtccta

SEQ ID NO: 457 - protein, 111 aa, Homo sapiens
SIELTQPPSVSVSPGQTASITCSGDNLGKKYVQWYQQKPGQSPVLVIYDDSNRPSGIPERFSGSNSGNTATLTISGTQAMDEADYYCQHWHGHHHTGETNVFGGGTKLTVL

SEQ ID NO: 458 - protein, 11 aa, Homo sapiens
SGDNLGKKYVQ

SEQ ID NO: 459 - protein, 7 aa, Homo sapiens
DDSNRPS

SEQ ID NO: 460 - protein, 14 aa, Homo sapiens
QHWHGHHHTGETNV

SEQ ID NO: 461 - DNA, 351 bp, Homo sapiens
  source 1..351: mol_type = other DNA
gaggtgcagctgctcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatccactacgccgacagcgtgaagggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 462 - protein, 117 aa, Homo sapiens
EVQLLESGGGLVQPGGSLRLSCAASGFTFSSYAMNWVRQAPGKGLEWVSTISYSGSLIHYADSVKGRFTISRDNSKNTLYLQMNSLRAEDTAVYYCARINHDPMDVWGQGTLVTVSS

SEQ ID NO: 463 - protein, 5 aa, Homo sapiens
SYAMN

SEQ ID NO: 464 - protein, 17 aa, Homo sapiens
TISYSGSLIHYADSVKG

SEQ ID NO: 465 - protein, 8 aa, Homo sapiens
INHDPMDV

SEQ ID NO: 466 - DNA, 333 bp, Homo sapiens; identical to SEQ ID NO: 6
  source 1..333: mol_type = other DNA

SEQ ID NO: 467 - protein, 111 aa, Homo sapiens; identical to SEQ ID NO: 7

SEQ ID NO: 468 - protein, 11 aa, Homo sapiens
SGDNLGKKYVQ

SEQ ID NO: 469 - protein, 7 aa, Homo sapiens
DDSNRPS

SEQ ID NO: 470 - protein, 14 aa, Homo sapiens
QTWDGNPTTGETNV

SEQ ID NO: 471 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcaccacatcagccaccacgacagccatcatggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 472 - protein, 117 aa, Homo sapiens
EVQLLESGGGLVQPGGSLRLSCAASGFTFSSYAMNWVRQAPGKGLEWVSTISHSGHHISHHDSHHGRFTISRDNSKNTLYLQMNSLRAEDTAVYYCARINHDPMDVWGQGTLVTVSS

SEQ ID NO: 473 - protein, 5 aa, Homo sapiens
SYAMN

SEQ ID NO: 474 - protein, 17 aa, Homo sapiens
TISHSGHHISHHDSHHG

SEQ ID NO: 475 - protein, 8 aa, Homo sapiens
INHDPMDV

SEQ ID NO: 476 - DNA, 333 bp, Homo sapiens; identical to SEQ ID NO: 16
  source 1..333: mol_type = other DNA

SEQ ID NO: 477 - protein, 111 aa, Homo sapiens; identical to SEQ ID NO: 7

SEQ ID NO: 478 - protein, 11 aa, Homo sapiens
SGDNLGKKYVQ

SEQ ID NO: 479 - protein, 7 aa, Homo sapiens
DDSNRPS

SEQ ID NO: 480 - protein, 14 aa, Homo sapiens
QTWDGNPTTGETNV

SEQ ID NO: 481 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcacctgatccaccacgccgacagccataagggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 482 - protein, 117 aa, Homo sapiens
EVQLLESGGGLVQPGGSLRLSCAASGFTFSSYAMNWVRQAPGKGLEWVSTISHSGHLIHHADSHKGRFTISRDNSKNTLYLQMNSLRAEDTAVYYCARINHDPMDVWGQGTLVTVSS

SEQ ID NO: 483 - protein, 5 aa, Homo sapiens
SYAMN

SEQ ID NO: 484 - protein, 17 aa, Homo sapiens
TISHSGHLIHHADSHKG

SEQ ID NO: 485 - protein, 8 aa, Homo sapiens
INHDPMDV

SEQ ID NO: 486 - DNA, 333 bp, Homo sapiens; identical to SEQ ID NO: 16
  source 1..333: mol_type = other DNA

SEQ ID NO: 487 - protein, 111 aa, Homo sapiens; identical to SEQ ID NO: 7

SEQ ID NO: 488 - protein, 11 aa, Homo sapiens
SGDNLGKKYVQ

SEQ ID NO: 489 - protein, 7 aa, Homo sapiens
DDSNRPS

SEQ ID NO: 490 - protein, 14 aa, Homo sapiens
QTWDGNPTTGETNV

SEQ ID NO: 491 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggcacctgatccactacgccgaccaccatcatggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 492 - protein, 117 aa, Homo sapiens
EVQLLESGGGLVQPGGSLRLSCAASGFTFSSYAMNWVRQAPGKGLEWVSTISYSGHLIHYADHHHGRFTISRDNSKNTLYLQMNSLRAEDTAVYYCARINHDPMDVWGQGTLVTVSS

SEQ ID NO: 493 - protein, 5 aa, Homo sapiens
SYAMN

SEQ ID NO: 494 - protein, 17 aa, Homo sapiens
TISYSGHLIHYADHHHG

SEQ ID NO: 495 - protein, 8 aa, Homo sapiens
INHDPMDV

SEQ ID NO: 496 - DNA, 333 bp, Homo sapiens; identical to SEQ ID NO: 16
  source 1..333: mol_type = other DNA

SEQ ID NO: 497 - protein, 111 aa, Homo sapiens; identical to SEQ ID NO: 7

SEQ ID NO: 498 - protein, 11 aa, Homo sapiens
SGDNLGKKYVQ

SEQ ID NO: 499 - protein, 7 aa, Homo sapiens
DDSNRPS

SEQ ID NO: 500 - protein, 14 aa, Homo sapiens
QTWDGNPTTGETNV

SEQ ID NO: 501 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagccacgccgaccacgtgcatggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 502 - protein, 117 aa, Homo sapiens
EVQLLESGGGLVQPGGSLRLSCAASGFTFSSYAMNWVRQAPGKGLEWVSTISYHGSHISHADHVHGRFTISRDNSKNTLYLQMNSLRAEDTAVYYCARINHDPMDVWGQGTLVTVSS

SEQ ID NO: 503 - protein, 5 aa, Homo sapiens
SYAMN

SEQ ID NO: 504 - protein, 17 aa, Homo sapiens
TISYHGSHISHADHVHG

SEQ ID NO: 505 - protein, 8 aa, Homo sapiens
INHDPMDV

SEQ ID NO: 506 - DNA, 333 bp, Homo sapiens; identical to SEQ ID NO: 16
  source 1..333: mol_type = other DNA

SEQ ID NO: 507 - protein, 111 aa, Homo sapiens; identical to SEQ ID NO: 7

SEQ ID NO: 508 - protein, 11 aa, Homo sapiens
SGDNLGKKYVQ

SEQ ID NO: 509 - protein, 7 aa, Homo sapiens
DDSNRPS

SEQ ID NO: 510 - protein, 14 aa, Homo sapiens
QTWDGNPTTGETNV

SEQ ID NO: 511 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagccatcatggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 512 - protein, 117 aa, Homo sapiens
EVQLLESGGGLVQPGGSLRLSCAASGFTFSSYAMNWVRQAPGKGLEWVSTISYHGSHISYHDSHHGRFTISRDNSKNTLYLQMNSLRAEDTAVYYCARINHDPMDVWGQGTLVTVSS

SEQ ID NO: 513 - protein, 5 aa, Homo sapiens
SYAMN

SEQ ID NO: 514 - protein, 17 aa, Homo sapiens
TISYHGSHISYHDSHHG

SEQ ID NO: 515 - protein, 8 aa, Homo sapiens
INHDPMDV

SEQ ID NO: 516 - DNA, 333 bp, Homo sapiens; identical to SEQ ID NO: 16
  source 1..333: mol_type = other DNA

SEQ ID NO: 517 - protein, 111 aa, Homo sapiens; identical to SEQ ID NO: 7

SEQ ID NO: 518 - protein, 11 aa, Homo sapiens
SGDNLGKKYVQ

SEQ ID NO: 519 - protein, 7 aa, Homo sapiens
DDSNRPS

SEQ ID NO: 520 - protein, 14 aa, Homo sapiens
QTWDGNPTTGETNV

SEQ ID NO: 521 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagctacgccgacagccatcatcacaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 522 - protein, 117 aa, Homo sapiens
EVQLLESGGGLVQPGGSLRLSCAASGFTFSSYAMNWVRQAPGKGLEWVSTISYHGHHISYADSHHHRFTISRDNSKNTLYLQMNSLRAEDTAVYYCARINHDPMDVWGQGTLVTVSS

SEQ ID NO: 523 - protein, 5 aa, Homo sapiens
SYAMN

SEQ ID NO: 524 - protein, 17 aa, Homo sapiens
TISYHGHHISYADSHHH

SEQ ID NO: 525 - protein, 8 aa, Homo sapiens
INHDPMDV

SEQ ID NO: 526 - DNA, 333 bp, Homo sapiens; identical to SEQ ID NO: 16
  source 1..333: mol_type = other DNA

SEQ ID NO: 527 - protein, 111 aa, Homo sapiens; identical to SEQ ID NO: 7

SEQ ID NO: 528 - protein, 11 aa, Homo sapiens
SGDNLGKKYVQ

SEQ ID NO: 529 - protein, 7 aa, Homo sapiens
DDSNRPS

SEQ ID NO: 530 - protein, 14 aa, Homo sapiens
QTWDGNPTTGETNV

SEQ ID NO: 531 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgcatcacaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtgacagtgagctca

SEQ ID NO: 532 - protein, 117 aa, Homo sapiens
EVQLLESGGGLVQPGGSLRLSCAASGFTFSSYAMNWVRQAPGKGLEWVSTISYHGSHISYHDSVHHRFTISRDNSKNTLYLQMNSLRAEDTAVYYCARINHDPMDVWGQGTLVTVSS

SEQ ID NO: 533 - protein, 5 aa, Homo sapiens
SYAMN

SEQ ID NO: 534 - protein, 17 aa, Homo sapiens
TISYHGSHISYHDSVHH

SEQ ID NO: 535 - protein, 8 aa, Homo sapiens
INHDPMDV

SEQ ID NO: 536 - DNA, 333 bp, Homo sapiens; identical to SEQ ID NO: 16
  source 1..333: mol_type = other DNA

SEQ ID NO: 537 - protein, 111 aa, Homo sapiens; identical to SEQ ID NO: 7

SEQ ID NO: 538 - protein, 11 aa, Homo sapiens
SGDNLGKKYVQ

SEQ ID NO: 539 - protein, 7 aa, Homo sapiens
DDSNRPS

SEQ ID NO: 540 - protein, 14 aa, Homo sapiens
QTWDGNPTTGETNV

SEQ ID NO: 541 - DNA, 351 bp, Homo sapiens
  source 1..351: mol_type = other DNA
gaggtgcagctgctcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatccactacgccgacagcgtgaagggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 542 - protein, 117 aa, Homo sapiens
EVQLLESGGGLVQPGGSLRLSCAASGFTFSSYAMNWVRQAPGKGLEWVSTISYSGSLIHYADSVKGRFTISRDNSKNTLYLQMNSLRAEDTAVYYCARIHHDPMDVWGQGTLVTVSS

SEQ ID NO: 543 - protein, 5 aa, Homo sapiens
SYAMN

SEQ ID NO: 544 - protein, 17 aa, Homo sapiens
TISYSGSLIHYADSVKG

SEQ ID NO: 545 - protein, 8 aa, Homo sapiens
IHHDPMDV

SEQ ID NO: 546 - DNA, 333 bp, Homo sapiens; identical to SEQ ID NO: 6
  source 1..333: mol_type = other DNA

SEQ ID NO: 547 - protein, 111 aa, Homo sapiens; identical to SEQ ID NO: 7

SEQ ID NO: 548 - protein, 11 aa, Homo sapiens
SGDNLGKKYVQ

SEQ ID NO: 549 - protein, 7 aa, Homo sapiens
DDSNRPS

SEQ ID NO: 550 - protein, 14 aa, Homo sapiens
QTWDGNPTTGETNV

SEQ ID NO: 551 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagccacagcgggcaccacatcagccaccacgacagccatcatggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 552 - protein, 117 aa, Homo sapiens
EVQLLESGGGLVQPGGSLRLSCAASGFTFSSYAMNWVRQAPGKGLEWVSTISHSGHHISHHDSHHGRFTISRDNSKNTLYLQMNSLRAEDTAVYYCARIHHDPMDVWGQGTLVTVSS

SEQ ID NO: 553 - protein, 5 aa, Homo sapiens
SYAMN

SEQ ID NO: 554 - protein, 17 aa, Homo sapiens
TISHSGHHISHHDSHHG

SEQ ID NO: 555 - protein, 8 aa, Homo sapiens
IHHDPMDV

SEQ ID NO: 556 - DNA, 333 bp, Homo sapiens; identical to SEQ ID NO: 16
  source 1..333: mol_type = other DNA

SEQ ID NO: 557 - protein, 111 aa, Homo sapiens; identical to SEQ ID NO: 7

SEQ ID NO: 558 - protein, 11 aa, Homo sapiens
SGDNLGKKYVQ

SEQ ID NO: 559 - protein, 7 aa, Homo sapiens
DDSNRPS

SEQ ID NO: 560 - protein, 14 aa, Homo sapiens
QTWDGNPTTGETNV

SEQ ID NO: 561 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggcacctgatccactacgccgaccaccatcatggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 562 - protein, 117 aa, Homo sapiens
EVQLLESGGGLVQPGGSLRLSCAASGFTFSSYAMNWVRQAPGKGLEWVSTISYSGHLIHYADHHHGRFTISRDNSKNTLYLQMNSLRAEDTAVYYCARIHHDPMDVWGQGTLVTVSS

SEQ ID NO: 563 - protein, 5 aa, Homo sapiens
SYAMN

SEQ ID NO: 564 - protein, 17 aa, Homo sapiens
TISYSGHLIHYADHHHG

SEQ ID NO: 565 - protein, 8 aa, Homo sapiens
IHHDPMDV

SEQ ID NO: 566 - DNA, 333 bp, Homo sapiens; identical to SEQ ID NO: 16
  source 1..333: mol_type = other DNA

SEQ ID NO: 567 - protein, 111 aa, Homo sapiens; identical to SEQ ID NO: 7

SEQ ID NO: 568 - protein, 11 aa, Homo sapiens
SGDNLGKKYVQ

SEQ ID NO: 569 - protein, 7 aa, Homo sapiens
DDSNRPS

SEQ ID NO: 570 - protein, 14 aa, Homo sapiens
QTWDGNPTTGETNV

SEQ ID NO: 571 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagccacgccgaccacgtgcatggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 572 - protein, 117 aa, Homo sapiens
EVQLLESGGGLVQPGGSLRLSCAASGFTFSSYAMNWVRQAPGKGLEWVSTISYHGSHISHADHVHGRFTISRDNSKNTLYLQMNSLRAEDTAVYYCARIHHDPMDVWGQGTLVTVSS

SEQ ID NO: 573 - protein, 5 aa, Homo sapiens
SYAMN

SEQ ID NO: 574 - protein, 17 aa, Homo sapiens
TISYHGSHISHADHVHG

SEQ ID NO: 575 - protein, 8 aa, Homo sapiens
IHHDPMDV

SEQ ID NO: 576 - DNA, 333 bp, Homo sapiens; identical to SEQ ID NO: 16
  source 1..333: mol_type = other DNA

SEQ ID NO: 577 - protein, 111 aa, Homo sapiens; identical to SEQ ID NO: 7

SEQ ID NO: 578 - protein, 11 aa, Homo sapiens
SGDNLGKKYVQ

SEQ ID NO: 579 - protein, 7 aa, Homo sapiens
DDSNRPS

SEQ ID NO: 580 - protein, 14 aa, Homo sapiens
QTWDGNPTTGETNV

SEQ ID NO: 581 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagccatcatggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 582 - protein, 117 aa, Homo sapiens
EVQLLESGGGLVQPGGSLRLSCAASGFTFSSYAMNWVRQAPGKGLEWVSTISYHGSHISYHDSHHGRFTISRDNSKNTLYLQMNSLRAEDTAVYYCARIHHDPMDVWGQGTLVTVSS

SEQ ID NO: 583 - protein, 5 aa, Homo sapiens
SYAMN

SEQ ID NO: 584 - protein, 17 aa, Homo sapiens
TISYHGSHISYHDSHHG

SEQ ID NO: 585 - protein, 8 aa, Homo sapiens
IHHDPMDV

SEQ ID NO: 586 - DNA, 333 bp, Homo sapiens; identical to SEQ ID NO: 16
  source 1..333: mol_type = other DNA

SEQ ID NO: 587 - protein, 111 aa, Homo sapiens; identical to SEQ ID NO: 7

SEQ ID NO: 588 - protein, 11 aa, Homo sapiens
SGDNLGKKYVQ

SEQ ID NO: 589 - protein, 7 aa, Homo sapiens
DDSNRPS

SEQ ID NO: 590 - protein, 14 aa, Homo sapiens
QTWDGNPTTGETNV

SEQ ID NO: 591 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagctacgccgacagccatcatcacaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 592 - protein, 117 aa, Homo sapiens
EVQLLESGGGLVQPGGSLRLSCAASGFTFSSYAMNWVRQAPGKGLEWVSTISYHGHHISYADSHHHRFTISRDNSKNTLYLQMNSLRAEDTAVYYCARIHHDPMDVWGQGTLVTVSS

SEQ ID NO: 593 - protein, 5 aa, Homo sapiens
SYAMN

SEQ ID NO: 594 - protein, 17 aa, Homo sapiens
TISYHGHHISYADSHHH

SEQ ID NO: 595 - protein, 8 aa, Homo sapiens
IHHDPMDV

SEQ ID NO: 596 - DNA, 333 bp, Homo sapiens; identical to SEQ ID NO: 16
  source 1..333: mol_type = other DNA

SEQ ID NO: 597 - protein, 111 aa, Homo sapiens; identical to SEQ ID NO: 7

SEQ ID NO: 598 - protein, 11 aa, Homo sapiens
SGDNLGKKYVQ

SEQ ID NO: 599 - protein, 7 aa, Homo sapiens
DDSNRPS

SEQ ID NO: 600 - protein, 14 aa, Homo sapiens
QTWDGNPTTGETNV

SEQ ID NO: 601 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagctacgccgacagccatcatcacaggttcaccatcagcagggacaacagcaagaacaccctgtacctgcagatgaacagcctgagggccgaggacaccgccgtgtactactgcgcccgcatccaccacgaccctatggatcactggggccagggcaccctggtgacagtgagctca

SEQ ID NO: 602 - protein, 117 aa, Homo sapiens
EVQLLESGGGLVQPGGSLRLSCAASGFTFSSYAMNWVRQAPGKGLEWVSTISYHGHHISYADSHHHRFTISRDNSKNTLYLQMNSLRAEDTAVYYCARIHHDPMDHWGQGTLVTVSS

SEQ ID NO: 603 - protein, 5 aa, Homo sapiens
SYAMN

SEQ ID NO: 604 - protein, 17 aa, Homo sapiens
TISYHGHHISYADSHHH

SEQ ID NO: 605 - protein, 8 aa, Homo sapiens
IHHDPMDH

SEQ ID NO: 606 - DNA, 333 bp, Homo sapiens; identical to SEQ ID NO: 16
  source 1..333: mol_type = other DNA

SEQ ID NO: 607 - protein, 111 aa, Homo sapiens; identical to SEQ ID NO: 7

SEQ ID NO: 608 - protein, 11 aa, Homo sapiens
SGDNLGKKYVQ

SEQ ID NO: 609 - protein, 7 aa, Homo sapiens
DDSNRPS

SEQ ID NO: 610 - protein, 14 aa, Homo sapiens
QTWDGNPTTGETNV

SEQ ID NO: 611 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgcatcacaggttcaccatcagcagggacaacagcaagaacaccctgtacctgcagatgaacagcctgagggccgaggacaccgccgtgtactactgcgcccgcatccaccacgaccctatggatcactggggccagggcaccctggtgacagtgagctca

SEQ ID NO: 612 - protein, 117 aa, Homo sapiens
EVQLLESGGGLVQPGGSLRLSCAASGFTFSSYAMNWVRQAPGKGLEWVSTISYHGSHISYHDSVHHRFTISRDNSKNTLYLQMNSLRAEDTAVYYCARIHHDPMDHWGQGTLVTVSS

SEQ ID NO: 613 - protein, 5 aa, Homo sapiens
SYAMN

SEQ ID NO: 614 - protein, 17 aa, Homo sapiens
TISYHGSHISYHDSVHH

SEQ ID NO: 615 - protein, 8 aa, Homo sapiens
IHHDPMDH

SEQ ID NO: 616 - DNA, 333 bp, Homo sapiens; identical to SEQ ID NO: 16
  source 1..333: mol_type = other DNA

SEQ ID NO: 617 - protein, 111 aa, Homo sapiens; identical to SEQ ID NO: 7

SEQ ID NO: 618 - protein, 11 aa, Homo sapiens
SGDNLGKKYVQ

SEQ ID NO: 619 - protein, 7 aa, Homo sapiens
DDSNRPS

SEQ ID NO: 620 - protein, 14 aa, Homo sapiens
QTWDGNPTTGETNV

SEQ ID NO: 621 - DNA, 351 bp, Homo sapiens
  source 1..351: mol_type = other DNA
gaggtgcagctgctcgagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtcaccgtctcctca

SEQ ID NO: 622 - protein, 117 aa, Homo sapiens; identical to SEQ ID NO: 32

SEQ ID NO: 623 - protein, 5 aa, Homo sapiens
SYAMN

SEQ ID NO: 624 - protein, 17 aa, Homo sapiens
TISYSGSLISYADSVKG

SEQ ID NO: 625 - protein, 8 aa, Homo sapiens
INHDPMDV

SEQ ID NO: 626 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggccatggacgaggccgactactactgccagacctgggacgggaacccgacgcacggcgagaccaacgtgttcggcggagggaccaagctgaccgtccta

SEQ ID NO: 627 - protein, 111 aa, Homo sapiens; identical to SEQ ID NO: 47

SEQ ID NO: 628 - protein, 11 aa, Homo sapiens
SGDNLGKKYVQ

SEQ ID NO: 629 - protein, 7 aa, Homo sapiens
DDSNRPS

SEQ ID NO: 630 - protein, 14 aa, Homo sapiens
QTWDGNPTHGETNV

SEQ ID NO: 631 - DNA, 351 bp, Homo sapiens
  source 1..351: mol_type = other DNA
gaggtgcagctgttggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctacagcgggtcgctgatcagctacgccgacagcgtgaagggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtcaccgtctcctca

SEQ ID NO: 632 - protein, 117 aa, Homo sapiens; identical to SEQ ID NO: 52

SEQ ID NO: 633 - protein, 5 aa, Homo sapiens
SYAMN

SEQ ID NO: 634 - protein, 17 aa, Homo sapiens
TISYSGSLISYADSVKG

SEQ ID NO: 635 - protein, 8 aa, Homo sapiens
IHHDPMDV

SEQ ID NO: 636 - DNA, 333 bp, Homo sapiens; identical to SEQ ID NO: 626
  source 1..333: mol_type = other DNA

SEQ ID NO: 637 - protein, 111 aa, Homo sapiens; identical to SEQ ID NO: 47

SEQ ID NO: 638 - protein, 11 aa, Homo sapiens
SGDNLGKKYVQ

SEQ ID NO: 639 - protein, 7 aa, Homo sapiens
DDSNRPS

SEQ ID NO: 640 - protein, 14 aa, Homo sapiens
QTWDGNPTHGETNV

SEQ ID NO: 641 - DNA, 351 bp, Homo sapiens
  source 1..351: mol_type = other DNA
gaggtgcagctgttggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagctacgccgacagccatcatcacaggttcaccatcagcagggacaacagcaagaacaccctgtacctgcagatgaacagcctgagggccgaggacaccgccgtgtactactgcgcccgcatcaacaacgaccctatggatgtgtggggccagggcaccctggtcaccgtctcctca

SEQ ID NO: 642 - protein, 117 aa, Homo sapiens; identical to SEQ ID NO: 332

SEQ ID NO: 643 - protein, 5 aa, Homo sapiens
SYAMN

SEQ ID NO: 644 - protein, 17 aa, Homo sapiens
TISYHGHHISYADSHHH

SEQ ID NO: 645 - protein, 8 aa, Homo sapiens
INNDPMDV

SEQ ID NO: 646 - DNA, 333 bp, Homo sapiens; identical to SEQ ID NO: 626
  source 1..333: mol_type = other DNA

SEQ ID NO: 647 - protein, 111 aa, Homo sapiens; identical to SEQ ID NO: 47

SEQ ID NO: 648 - protein, 11 aa, Homo sapiens
SGDNLGKKYVQ

SEQ ID NO: 649 - protein, 7 aa, Homo sapiens
DDSNRPS

SEQ ID NO: 650 - protein, 14 aa, Homo sapiens
QTWDGNPTHGETNV

SEQ ID NO: 651 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggcaccacatcagctacgccgacagccatcatcacaggttcaccatcagcagggacaacagcaagaacaccctgtacctgcagatgaacagcctgagggccgaggacaccgccgtgtactactgcgcccgcatcaaccacgaccctatggatgtgtggggccagggcaccctggtcaccgtctcctca

SEQ ID NO: 652 - protein, 117 aa, Homo sapiens; identical to SEQ ID NO: 522

SEQ ID NO: 653 - protein, 5 aa, Homo sapiens
SYAMN

SEQ ID NO: 654 - protein, 17 aa, Homo sapiens
TISYHGHHISYADSHHH

SEQ ID NO: 655 - protein, 8 aa, Homo sapiens
INHDPMDV

SEQ ID NO: 656 - DNA, 333 bp, Homo sapiens; identical to SEQ ID NO: 626
  source 1..333: mol_type = other DNA

SEQ ID NO: 657 - protein, 111 aa, Homo sapiens; identical to SEQ ID NO: 47

SEQ ID NO: 658 - protein, 11 aa, Homo sapiens
SGDNLGKKYVQ

SEQ ID NO: 659 - protein, 7 aa, Homo sapiens
DDSNRPS

SEQ ID NO: 660 - protein, 14 aa, Homo sapiens
QTWDGNPTHGETNV

SEQ ID NO: 661 - DNA, 351 bp, Homo sapiens; identical to SEQ ID NO: 621
  source 1..351: mol_type = other DNA

SEQ ID NO: 662 - protein, 117 aa, Homo sapiens; identical to SEQ ID NO: 32

SEQ ID NO: 663 - protein, 5 aa, Homo sapiens
SYAMN

SEQ ID NO: 664 - protein, 17 aa, Homo sapiens
TISYSGSLISYADSVKG

SEQ ID NO: 665 - protein, 8 aa, Homo sapiens
INHDPMDV

SEQ ID NO: 666 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctggcaccaccacccgacgcatggggagcacaacgtgttcggcggagggaccaagctgaccgtccta

SEQ ID NO: 667 - protein, 111 aa, Homo sapiens; identical to SEQ ID NO: 97

SEQ ID NO: 668 - protein, 11 aa, Homo sapiens
SGDNLGKKYVQ

SEQ ID NO: 669 - protein, 7 aa, Homo sapiens
DDSNRPS

SEQ ID NO: 670 - protein, 14 aa, Homo sapiens
HTWHHHPTHGEHNV

SEQ ID NO: 671 - DNA, 351 bp, Homo sapiens; identical to SEQ ID NO: 641
  source 1..351: mol_type = other DNA

SEQ ID NO: 672 - protein, 117 aa, Homo sapiens; identical to SEQ ID NO: 332

SEQ ID NO: 673 - protein, 5 aa, Homo sapiens
SYAMN

SEQ ID NO: 674 - protein, 17 aa, Homo sapiens
TISYHGHHISYADSHHH

SEQ ID NO: 675 - protein, 8 aa, Homo sapiens
INNDPMDV

SEQ ID NO: 676 - DNA, 333 bp, Homo sapiens; identical to SEQ ID NO: 666
  source 1..333: mol_type = other DNA

SEQ ID NO: 677 - protein, 111 aa, Homo sapiens; identical to SEQ ID NO: 97

SEQ ID NO: 678 - protein, 11 aa, Homo sapiens
SGDNLGKKYVQ

SEQ ID NO: 679 - protein, 7 aa, Homo sapiens
DDSNRPS

SEQ ID NO: 680 - protein, 14 aa, Homo sapiens
HTWHHHPTHGEHNV

SEQ ID NO: 681 - DNA, 351 bp, Homo sapiens; identical to SEQ ID NO: 651
  source 1..351: mol_type = other DNA

SEQ ID NO: 682 - protein, 117 aa, Homo sapiens; identical to SEQ ID NO: 522

SEQ ID NO: 683 - protein, 5 aa, Homo sapiens
SYAMN

SEQ ID NO: 684 - protein, 17 aa, Homo sapiens
TISYHGHHISYADSHHH

SEQ ID NO: 685 - protein, 8 aa, Homo sapiens
INHDPMDV

SEQ ID NO: 686 - DNA, 333 bp, Homo sapiens; identical to SEQ ID NO: 666
  source 1..333: mol_type = other DNA

SEQ ID NO: 687 - protein, 111 aa, Homo sapiens; identical to SEQ ID NO: 97

SEQ ID NO: 688 - protein, 11 aa, Homo sapiens
SGDNLGKKYVQ

SEQ ID NO: 689 - protein, 7 aa, Homo sapiens
DDSNRPS

SEQ ID NO: 690 - protein, 14 aa, Homo sapiens
HTWHHHPTHGEHNV

SEQ ID NO: 691 - DNA, 351 bp, Homo sapiens; identical to SEQ ID NO: 621
  source 1..351: mol_type = other DNA

SEQ ID NO: 692 - protein, 117 aa, Homo sapiens; identical to SEQ ID NO: 32

SEQ ID NO: 693 - protein, 5 aa, Homo sapiens
SYAMN

SEQ ID NO: 694 - protein, 17 aa, Homo sapiens
TISYSGSLISYADSVKG

SEQ ID NO: 695 - protein, 8 aa, Homo sapiens
INHDPMDV

SEQ ID NO: 696 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctgggaccaccaccacacgacgggggagcacaacgtgttcggcggagggaccaagctgaccgtccta

SEQ ID NO: 697 - protein, 111 aa, Homo sapiens; identical to SEQ ID NO: 157

SEQ ID NO: 698 - protein, 11 aa, Homo sapiens
SGDNLGKKYVQ

SEQ ID NO: 699 - protein, 7 aa, Homo sapiens
DDSNRPS

SEQ ID NO: 700 - protein, 14 aa, Homo sapiens
HTWDHHHTTGEHNV

SEQ ID NO: 701 - DNA, 351 bp, Homo sapiens; identical to SEQ ID NO: 631
  source 1..351: mol_type = other DNA

SEQ ID NO: 702 - protein, 117 aa, Homo sapiens; identical to SEQ ID NO: 52

SEQ ID NO: 703 - protein, 5 aa, Homo sapiens
SYAMN

SEQ ID NO: 704 - protein, 17 aa, Homo sapiens
TISYSGSLISYADSVKG

SEQ ID NO: 705 - protein, 8 aa, Homo sapiens
IHHDPMDV

SEQ ID NO: 706 - DNA, 333 bp, Homo sapiens; identical to SEQ ID NO: 696
  source 1..333: mol_type = other DNA

SEQ ID NO: 707 - protein, 111 aa, Homo sapiens; identical to SEQ ID NO: 157

SEQ ID NO: 708 - protein, 11 aa, Homo sapiens
SGDNLGKKYVQ

SEQ ID NO: 709 - protein, 7 aa, Homo sapiens
DDSNRPS

SEQ ID NO: 710 - protein, 14 aa, Homo sapiens
HTWDHHHTTGEHNV

SEQ ID NO: 711 - DNA, 351 bp, Homo sapiens; identical to SEQ ID NO: 641
  source 1..351: mol_type = other DNA

SEQ ID NO: 712 - protein, 117 aa, Homo sapiens; identical to SEQ ID NO: 332

SEQ ID NO: 713 - protein, 5 aa, Homo sapiens
SYAMN

SEQ ID NO: 714 - protein, 17 aa, Homo sapiens
TISYHGHHISYADSHHH

SEQ ID NO: 715 - protein, 8 aa, Homo sapiens
INNDPMDV

SEQ ID NO: 716 - DNA, 333 bp, Homo sapiens; identical to SEQ ID NO: 696
  source 1..333: mol_type = other DNA

SEQ ID NO: 717 - protein, 111 aa, Homo sapiens; identical to SEQ ID NO: 157

SEQ ID NO: 718 - protein, 11 aa, Homo sapiens
SGDNLGKKYVQ

SEQ ID NO: 719 - protein, 7 aa, Homo sapiens
DDSNRPS

SEQ ID NO: 720 - protein, 14 aa, Homo sapiens
HTWDHHHTTGEHNV

SEQ ID NO: 721 - DNA, 351 bp, Homo sapiens; identical to SEQ ID NO: 621
  source 1..351: mol_type = other DNA

SEQ ID NO: 722 - protein, 117 aa, Homo sapiens; identical to SEQ ID NO: 32

SEQ ID NO: 723 - protein, 5 aa, Homo sapiens
SYAMN

SEQ ID NO: 724 - protein, 17 aa, Homo sapiens
TISYSGSLISYADSVKG

SEQ ID NO: 725 - protein, 8 aa, Homo sapiens
INHDPMDV

SEQ ID NO: 726 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccagacctgggacgggcaccaccacacgggggagaccaaccacttcggcggagggaccaagctgaccgtccta

SEQ ID NO: 727 - protein, 111 aa, Homo sapiens; identical to SEQ ID NO: 197

SEQ ID NO: 728 - protein, 11 aa, Homo sapiens
SGDNLGKKYVQ

SEQ ID NO: 729 - protein, 7 aa, Homo sapiens
DDSNRPS

SEQ ID NO: 730 - protein, 14 aa, Homo sapiens
QTWDGHHHTGETNH

SEQ ID NO: 731 - DNA, 351 bp, Homo sapiens; identical to SEQ ID NO: 631
  source 1..351: mol_type = other DNA

SEQ ID NO: 732 - protein, 117 aa, Homo sapiens; identical to SEQ ID NO: 52

SEQ ID NO: 733 - protein, 5 aa, Homo sapiens
SYAMN

SEQ ID NO: 734 - protein, 17 aa, Homo sapiens
TISYSGSLISYADSVKG

SEQ ID NO: 735 - protein, 8 aa, Homo sapiens
IHHDPMDV

SEQ ID NO: 736 - DNA, 333 bp, Homo sapiens; identical to SEQ ID NO: 726
  source 1..333: mol_type = other DNA

SEQ ID NO: 737 - protein, 111 aa, Homo sapiens; identical to SEQ ID NO: 197

SEQ ID NO: 738 - protein, 11 aa, Homo sapiens
SGDNLGKKYVQ

SEQ ID NO: 739 - protein, 7 aa, Homo sapiens
DDSNRPS

SEQ ID NO: 740 - protein, 14 aa, Homo sapiens
QTWDGHHHTGETNH

SEQ ID NO: 741 - DNA, 351 bp, Homo sapiens; identical to SEQ ID NO: 641
  source 1..351: mol_type = other DNA

SEQ ID NO: 742 - protein, 117 aa, Homo sapiens; identical to SEQ ID NO: 332

SEQ ID NO: 743 - protein, 5 aa, Homo sapiens
SYAMN

SEQ ID NO: 744 - protein, 17 aa, Homo sapiens
TISYHGHHISYADSHHH

SEQ ID NO: 745 - protein, 8 aa, Homo sapiens
INNDPMDV

SEQ ID NO: 746 - DNA, 333 bp, Homo sapiens; identical to SEQ ID NO: 726
  source 1..333: mol_type = other DNA

SEQ ID NO: 747 - protein, 111 aa, Homo sapiens; identical to SEQ ID NO: 197

SEQ ID NO: 748 - protein, 11 aa, Homo sapiens
SGDNLGKKYVQ

SEQ ID NO: 749 - protein, 7 aa, Homo sapiens
DDSNRPS

SEQ ID NO: 750 - protein, 14 aa, Homo sapiens
QTWDGHHHTGETNH

SEQ ID NO: 751 - DNA, 351 bp, Homo sapiens; identical to SEQ ID NO: 651
  source 1..351: mol_type = other DNA

SEQ ID NO: 752 - protein, 117 aa, Homo sapiens; identical to SEQ ID NO: 522

SEQ ID NO: 753 - protein, 5 aa, Homo sapiens
SYAMN

SEQ ID NO: 754 - protein, 17 aa, Homo sapiens
TISYHGHHISYADSHHH

SEQ ID NO: 755 - protein, 8 aa, Homo sapiens
INHDPMDV

SEQ ID NO: 756 - DNA, 333 bp, Homo sapiens; identical to SEQ ID NO: 726
  source 1..333: mol_type = other DNA

SEQ ID NO: 757 - protein, 111 aa, Homo sapiens; identical to SEQ ID NO: 197

SEQ ID NO: 758 - protein, 11 aa, Homo sapiens
SGDNLGKKYVQ

SEQ ID NO: 759 - protein, 7 aa, Homo sapiens
DDSNRPS

SEQ ID NO: 760 - protein, 14 aa, Homo sapiens
QTWDGHHHTGETNH

SEQ ID NO: 761 - DNA, 351 bp, Homo sapiens; identical to SEQ ID NO: 621
  source 1..351: mol_type = other DNA

SEQ ID NO: 762 - protein, 117 aa, Homo sapiens; identical to SEQ ID NO: 32

SEQ ID NO: 763 - protein, 5 aa, Homo sapiens
SYAMN

SEQ ID NO: 764 - protein, 17 aa, Homo sapiens
TISYSGSLISYADSVKG

SEQ ID NO: 765 - protein, 8 aa, Homo sapiens
INHDPMDV

SEQ ID NO: 766 - DNA, 333 bp, Homo sapiens
  source 1..333: mol_type = other DNA
agtatcgagctgacccagccccccagcgtgagcgtgtccccaggccagaccgccagcatcacctgcagcggcgacaacctgggcaagaaatacgtgcagtggtatcagcagaagcccggccagtcccccgtgctggtgatctacgacgacagcaacaggcccagcggcatccccgagaggttcagcggcagcaacagcggcaacaccgccaccctgaccatcagcggcacccaggcgatggacgaggccgactactactgccacacctgggacgggcaccacacgacgggggagaccaaccacttcggcggagggaccaagctgaccgtccta

SEQ ID NO: 767 - protein, 111 aa, Homo sapiens; identical to SEQ ID NO: 407

SEQ ID NO: 768 - protein, 11 aa, Homo sapiens
SGDNLGKKYVQ

SEQ ID NO: 769 - protein, 7 aa, Homo sapiens
DDSNRPS

SEQ ID NO: 770 - protein, 14 aa, Homo sapiens
HTWDGHHTTGETNH

SEQ ID NO: 771 - DNA, 351 bp, Homo sapiens; identical to SEQ ID NO: 631
  source 1..351: mol_type = other DNA

SEQ ID NO: 772 - protein, 117 aa, Homo sapiens; identical to SEQ ID NO: 52

SEQ ID NO: 773 - protein, 5 aa, Homo sapiens
SYAMN

SEQ ID NO: 774 - protein, 17 aa, Homo sapiens
TISYSGSLISYADSVKG

SEQ ID NO: 775 - protein, 8 aa, Homo sapiens
IHHDPMDV

SEQ ID NO: 776 - DNA, 333 bp, Homo sapiens; identical to SEQ ID NO: 766
  source 1..333: mol_type = other DNA

SEQ ID NO: 777 - protein, 111 aa, Homo sapiens; identical to SEQ ID NO: 407

SEQ ID NO: 778 - protein, 11 aa, Homo sapiens
SGDNLGKKYVQ

SEQ ID NO: 779 - protein, 7 aa, Homo sapiens
DDSNRPS

SEQ ID NO: 780 - protein, 14 aa, Homo sapiens
HTWDGHHTTGETNH

SEQ ID NO: 781 - DNA, 351 bp, Homo sapiens; identical to SEQ ID NO: 641
  source 1..351: mol_type = other DNA

SEQ ID NO: 782 - protein, 117 aa, Homo sapiens; identical to SEQ ID NO: 332

SEQ ID NO: 783 - protein, 5 aa, Homo sapiens
SYAMN

SEQ ID NO: 784 - protein, 17 aa, Homo sapiens
TISYHGHHISYADSHHH

SEQ ID NO: 785 - protein, 8 aa, Homo sapiens
INNDPMDV

SEQ ID NO: 786 - DNA, 333 bp, Homo sapiens; identical to SEQ ID NO: 766
  source 1..333: mol_type = other DNA

SEQ ID NO: 787 - protein, 111 aa, Homo sapiens; identical to SEQ ID NO: 407

SEQ ID NO: 788 - protein, 11 aa, Homo sapiens
SGDNLGKKYVQ

SEQ ID NO: 789 - protein, 7 aa, Homo sapiens
DDSNRPS

SEQ ID NO: 790 - protein, 14 aa, Homo sapiens
HTWDGHHTTGETNH

SEQ ID NO: 791 - DNA, 351 bp, Homo sapiens; identical to SEQ ID NO: 651
  source 1..351: mol_type = other DNA

SEQ ID NO: 792 - protein, 117 aa, Homo sapiens; identical to SEQ ID NO: 522

SEQ ID NO: 793 - protein, 5 aa, Homo sapiens
SYAMN

SEQ ID NO: 794 - protein, 17 aa, Homo sapiens
TISYHGHHISYADSHHH

SEQ ID NO: 795 - protein, 8 aa, Homo sapiens
INHDPMDV

SEQ ID NO: 796 - DNA, 333 bp, Homo sapiens; identical to SEQ ID NO: 766
  source 1..333: mol_type = other DNA

SEQ ID NO: 797 - protein, 111 aa, Homo sapiens; identical to SEQ ID NO: 407

SEQ ID NO: 798 - protein, 11 aa, Homo sapiens
SGDNLGKKYVQ

SEQ ID NO: 799 - protein, 7 aa, Homo sapiens
DDSNRPS

SEQ ID NO: 800 - protein, 14 aa, Homo sapiens
HTWDGHHTTGETNH

SEQ ID NO: 801 - protein, 397 aa, Homo sapiens
MRSPSAAWLLGAAILLAASLSCSGTIQGTNRSSKGRSLIGKVDGTSHVTGKGVTVETVFSVDEFSASVLTGKLTTVFLPIVYTIVFVVGLPSNGMALWVFLFRTKKKHPAVIYMANLALADLLSVIWFPLKIAYHIHGNNWIYGEALCNVLIGFFYGNMYCSILFMTCLSVQRYWVIVNPMGHSRKKANIAIGISLAIWLLILLVTIPLYVVKQTIFIPALNITTCHDVLPEQLLVGDMFNYFLSLAIGVFLFPAFLTASAYVLMIRMLRSSAMDENSEKKRKRAIKLIVTVLAMYLICFTPSNLLLVVHYFLIKSQGQSHVYALYIVALCLSTLNSCIDPFVYYFVSHDFRDHAKNALLCRSVRTVKQMQVSLTSKKHSRKSSSYSSSSTTVKTSY

SEQ ID NO: 802 - protein, 39 aa, Homo sapiens
SLIGKVDGTSHVTGKGVTVETVFSVDEFSASVLTGKLTT

SEQ ID NO: 803 - protein, 30 aa, Homo sapiens
EVQLLESGGGLVQPGGSLRLSCAASGFTFS

SEQ ID NO: 804 - protein, 14 aa, Homo sapiens
WVRQAPGKGLEWVS

SEQ ID NO: 805 - protein, 32 aa, Homo sapiens
RFTISRDNSKNTLYLQMNSLRAEDTAVYYCAR

SEQ ID NO: 806 - protein, 11 aa, Homo sapiens
WGQGTLVTVSS

SEQ ID NO: 807 - protein, 22 aa, Homo sapiens
SIELTQPPSVSVSPGQTASITC

SEQ ID NO: 808 - protein, 15 aa, Homo sapiens
WYQQKPGQSPVLVIY

SEQ ID NO: 809 - protein, 32 aa, Homo sapiens
GIPERFSGSNSGNTATLTISGTQAMDEADYYC

SEQ ID NO: 810 - protein, 10 aa, Homo sapiens
FGGGTKLTVL

SEQ ID NO: 811 - protein, 17 aa, Homo sapiens
TISYSGSHISYHDSVHH

SEQ ID NO: 812 - protein, 17 aa, Homo sapiens
TISYHGSLISYHDSVHH

SEQ ID NO: 813 - protein, 17 aa, Homo sapiens
TISYHGSHISYADSVHH

SEQ ID NO: 814 - protein, 17 aa, Homo sapiens
TISYHGSHISYHDSVKH

SEQ ID NO: 815 - protein, 17 aa, Homo sapiens
TISYHGSHISYHDSVHG

SEQ ID NO: 816 - protein, 17 aa, Homo sapiens
TISYHGSLISYADSVKG

SEQ ID NO: 817 - protein, 17 aa, Homo sapiens
TISYSGSHISYADSVKG

SEQ ID NO: 818 - protein, 17 aa, Homo sapiens
TISYHGSHISYADSVKG

SEQ ID NO: 819 - protein, 8 aa, Homo sapiens
IHNDPMDV

SEQ ID NO: 820 - protein, 8 aa, Homo sapiens
INHDPMDV

SEQ ID NO: 821 - protein, 117 aa, Homo sapiens; identical to SEQ ID NO: 12

SEQ ID NO: 822 - protein, 117 aa, Homo sapiens
EVQLLESGGGLVQPGGSLRLSCAASGFTFSSYAMNWVRQAPGKGLEWVSTISYSGSHISYHDSVHHRFTISRDNSKNTLYLQMNSLRAEDTAVYYCARIHHDPMDVWGQGTLVTVSS

SEQ ID NO: 823 - protein, 117 aa, Homo sapiens
EVQLLESGGGLVQPGGSLRLSCAASGFTFSSYAMNWVRQAPGKGLEWVSTISYHGSLISYHDSVHHRFTISRDNSKNTLYLQMNSLRAEDTAVYYCARIHHDPMDVWGQGTLVTVSS

SEQ ID NO: 824 - protein, 117 aa, Homo sapiens
EVQLLESGGGLVQPGGSLRLSCAASGFTFSSYAMNWVRQAPGKGLEWVSTISYHGSHISYADSVHHRFTISRDNSKNTLYLQMNSLRAEDTAVYYCARIHHDPMDVWGQGTLVTVSS

SEQ ID NO: 825 - protein, 117 aa, Homo sapiens
EVQLLESGGGLVQPGGSLRLSCAASGFTFSSYAMNWVRQAPGKGLEWVSTISYHGSHISYHDSVKHRFTISRDNSKNTLYLQMNSLRAEDTAVYYCARIHHDPMDVWGQGTLVTVSS

SEQ ID NO: 826 - protein, 117 aa, Homo sapiens
EVQLLESGGGLVQPGGSLRLSCAASGFTFSSYAMNWVRQAPGKGLEWVSTISYHGSHISYHDSVHGRFTISRDNSKNTLYLQMNSLRAEDTAVYYCARIHHDPMDVWGQGTLVTVSS

SEQ ID NO: 827 - protein, 117 aa, Homo sapiens; identical to SEQ ID NO: 532

SEQ ID NO: 828 - protein, 117 aa, Homo sapiens
EVQLLESGGGLVQPGGSLRLSCAASGFTFSSYAMNWVRQAPGKGLEWVSTISYHGSHISYHDSVHHRFTISRDNSKNTLYLQMNSLRAEDTAVYYCARIHNDPMDVWGQGTLVTVSS

SEQ ID NO: 829 - protein, 117 aa, Homo sapiens
EVQLLESGGGLVQPGGSLRLSCAASGFTFSSYAMNWVRQAPGKGLEWVSTISYHGSLISYADSVKGRFTISRDNSKNTLYLQMNSLRAEDTAVYYCARIHHDPMDVWGQGTLVTVSS

SEQ ID NO: 830 - protein, 117 aa, Homo sapiens
EVQLLESGGGLVQPGGSLRLSCAASGFTFSSYAMNWVRQAPGKGLEWVSTISYSGSHISYADSVKGRFTISRDNSKNTLYLQMNSLRAEDTAVYYCARIHHDPMDVWGQGTLVTVSS

SEQ ID NO: 831 - protein, 117 aa, Homo sapiens
EVQLLESGGGLVQPGGSLRLSCAASGFTFSSYAMNWVRQAPGKGLEWVSTISYHGSHISYADSVKGRFTISRDNSKNTLYLQMNSLRAEDTAVYYCARIHHDPMDVWGQGTLVTVSS

SEQ ID NO: 832 - protein, 6 aa, synthetic construct
  REGION 1..6: note = Synthetic peptide
  MOD_RES 6: note = Xaa is Ornithine
LIGRLX

SEQ ID NO: 833 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctactccgggtcgcacatcagctaccacgacagcgtgcatcacaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 834 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgctcatcagctaccacgacagcgtgcatcacaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 835 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctacgccgacagcgtgcatcacaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 836 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgaagcacaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 837 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgcatggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 838 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctaccacgacagcgtgcatcacaggttcaccatcagcagggacaacagcaagaacaccctgtacctgcagatgaacagcctgagggccgaggacaccgccgtgtactactgcgcccgcatccacaacgaccctatggatgtgtggggccagggcaccctggtgacagtgagctca

SEQ ID NO: 839 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgctcatcagctacgccgacagcgtgaagggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 840 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctactccgggtcgcacatcagctacgccgacagcgtgaagggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca

SEQ ID NO: 841 - DNA, 351 bp, Homo sapiens
  source 1..351: mol_type = other DNA
gaggtgcagctgctggagagcggcggagggctggtgcagccaggcggcagcctgaggctgtcctgcgccgcctccggattcaccttcagcagctacgccatgaactgggtgcggcaggccccaggcaagggcctggagtgggtgtccaccatcagctaccacgggtcgcacatcagctacgccgacagcgtgaagggcaggttcaccatcagcagggacaacagcaagaacaccctgtacctgcagatgaacagcctgagggccgaggacaccgccgtgtactactgcgcccgcatccaccacgaccctatggatgtgtggggccagggcaccctggtgacagtgagctca